(19) United States
(12) Patent Application Publication
(10) Pub. No.: US 2008/0311185 A1
(43) Pub. Date: Dec. 18, 2008

(54) Deregulated Genes and/or Processes in Inflammatory Arthritis

(76) Inventors: Georgios Kollias, Aghia Paraskevi (GR); Vassilios Aidinis, Nea Smurni (GR); Yoshishide Hayashizaki, Saitama (JP); Piero Carninci, Saitama (JP)

Correspondence Address: HOFFMANN & BARON, LLP, 6900 JERICHO TURNPIKE, SYOSSET, NY, 11791, US

(21) Appl. No.: 12/090,999

(22) PCT Filed: Oct. 19, 2006
(86) PCT No.: PCT/EP2006/010228

§ 371 Date: Aug. 5, 2008

(30) Foreign Application Priority Data
Oct. 19, 2005 (GR) 20050100526

(51) Int. Cl. A61K 31/7088; C12Q 1/68; A61K 9/127; A61K 36/06; A61K 38/12; A01K 67/027; G01N 33/53; A61K 38/00; A61P 19/02

(52) U.S. Cl. 424/450; 435/6; 514/44; 424/195.15; 514/9; 800/13; 800/9; 800/3; 514/12

(57) ABSTRACT

The invention provides means and methods for determining whether a sample is derived from an individual suffering from inflammatory arthritis or from an individual at risk of suffering there from, said method comprising measuring in said sample the level of expression of at least one gene product of a gene of table A, table B or and comparing said expression level with a reference value. Further provided is a non-human animal model comprising an altered expression of a gene of table A, table B or when compared to a reference non-human animal, and use of this model as or in an arthritis model. Further provided is an in vitro assay for screening compounds for effect on a phenotype of synovial fibroblasts. The synovial fibroblast are preferably derived from a human or non-human animal suffering from inflammatory arthritis.

The invention relates to the field of molecular diagnostics and medicine. The invention in particular relates to the deregulation of genes and/or processes in arthritis. The invention utilizes this deregulation in a number of ways.

The invention is exemplified by means of Rheumatoid arthritis (RA), however, the invention is not limited to this disease but is applicable to all inflammatory arthritis diseases. RA is a chronic destructive arthropathy that affects 1-3% of the general population. RA imposes substantial personal, social and economic costs. It is characterized by prolonged inflammation of the joints, eventually leading to destruction of the cartilage and bone. Inflammation is initially localized in the synovial lining, a monolayer of synovial cells that lines diarthoidal joints. In RA, the synovial lining becomes markedly thickened due to synovial cell proliferation and infiltration by inflammatory cells. This proliferative mass, the pannus, invades and destroys articular cartilage and bone, leading to irreversible destruction of joint structure and function [1]. Current therapies of RA rely mainly on symptomatic treatment with non-steroidal anti-inflammatory drugs and/or with disease-modifying anti-rheumatic drugs. However, even the best available treatments (like targeting TNF and TNF signaling) do not cure the disease and do not even sufficiently retard progression in the majority of the patients, while they often exhibit adverse side effects [2].

Despite intense efforts, the etiology and pathogenesis of RA remain poorly understood. Traditional paradigms for RA have implicated a variety of mechanisms that contribute to the initiation and perpetuation of synovial inflammation, including autoantibodies and immune complexes, T-cell mediated antigen-specific responses, persistence of cytokine networks and other pro-inflammatory molecules, genetic bias and sex predisposition, as well as proliferative behaviour of the arthritic synovium [3]. Animal models of RA share many clinical features with the human disease and hence constitute valuable tools in deciphering the pathogenetic mechanisms that govern disease activation and perpetuation [4]. Among them, the TNF-Tg mouse [5] has been instrumental in demonstrating the role of TNF in the development of the disease and foreshadowed the introduction and success of anti-TNF therapies that transformed the effective management of the disease [6]. In this model, chronic over expression of human TNF results in a chronic, erosive, symmetric polyarthritis, with 100% phenotypic penetrance, timed disease onset and progressive histological symptoms that resemble closely human RA [5-7].

The completion of the Human Genome Project and the emergence of functional genomics and high-throughput technologies offer new possibilities for research into the etiology of RA. Amongst them, expression profiling creates a molecular fingerprint of the disease status, by the simultaneous quantification of the expression levels of thousand of genes. Similarly, reverse genetics (the genetic modification of a particular gene in a search of its function) allow for the creation of animal models of disease, valuable tools for the dissection of pathogenetic mechanisms in vivo.

Expression profiling, the relative quantification of the expression levels of thousand of genes simultaneously, is an approach for understanding mechanisms of differentiation, development and disease. However, the small number of samples employed limits significantly statistical analysis and precludes the use of False Discovery Rates methodologies, as well as of multivariate techniques, which would be appropriate for polygenic diseases such as RA. As it is, results of expression profiling need to be confirmed by an independent biological method, which largely diminishes the high throughput nature and discovery rate of any given approach.

To overcome these problems the invention in one aspect provides a novel, twin high throughput approach, composed of subtractive cDNA libraries and oligonucleotide DNA microarray hybridizations. Both methodologies, due to experimental setup, are governed by different constraints: random chance for the libraries, robotic clone selection and chip design for the microarrays. Consequently, both methodologies employ different, independent statistical analyses, are completely uncorrelated towards the propagation of error and conceptually have entirely different False Discovery Rates. As a result of this, the intersection of their statistically significant deregulated gene lists has a very low false discovery rate. To illustrate this and to exhibit the validity of the twin high throughput approach, a number of representative genes from the commonly selected list was confirmed with a number of different methods, such as automated literature search, semi-quantitative and quantitative RT-PCR in both mouse and human samples. Thus is illustrated that coupling of two different high throughput approaches renders independent confirmation unnecessary, although it can of course still be done in addition. Consequently the number of likely deregulated genes is increased. In one embodiment the invention thus provides a method for identifying genes involved in a disorder, comprising performing separately a subtractive cDNA library methodology and a oligonucleotide DNA microarray hybridization methodology and optionally another high throughput expression profiling methodology on a sample of an individual and then taking an intersection of results of said methodologies.

To validate the generated deregulated gene list even further, as well as to bridge two different animal models of RA, expression profiling in the invention was combined with genetic linkage analysis. All differentially expressed genes were mapped to the chromosomes, together with the known QTLs for an induced animal model of Arthritis, Collagen Induced Arthritis (CIA)[14]. Eight deregulated genes map to CIA QTLs (FIG. 1; WJ: Ctss, Pitpnm, Ncf1, Psmb8, Siat8e, SF: Rab14, Aqp1, and Gsn), leading to the conclusion that these genes have a dominant influence in arthritic processes, irrespective of the inciting stimulus (autoimmune or inflammatory). Interestingly, several of the genes cluster together in adjacent regions of the chromosomes (4, 7, 10, 11 and 13). These loci could define new QTLs or imply common regulatory control at the chromatin level.

Deregulated genes express their one or more gene products (or their enzymatic activities) either at a lower or at a higher level when compared to “normal cells” obtained from a healthy individual. This altered expression can be used to scrutinize samples and determine whether a sample is from an individual suffering from inflammatory arthritis (IA) or is at risk of suffering from IA. The invention thus provides a method for determining whether a sample is derived from an individual suffering from inflammation arthritis or from an individual at risk of suffering from IA, said method comprising measuring in said sample the level of expression or enzymatic activity of at least one gene product of a gene of table A, table B, or table 4 and comparing said expression level with a reference value. The mentioned tables indicate whether in IA, expression or enzymatic activity of said gene product is higher than “normal” or lower than “normal”. Thus by measuring the expression level or enzymatic activity of at least one gene product, and comparing it with a reference value, one can identify the sample as being derived from an individual suffering from IA, or at risk of suffering from IA, or derived from an individual that is not suffering from IA or is not at risk of suffering from IA. It is possible to identify a sample in this way by analyzing expression of gene products or their enzymatic activity of one of said genes. In this case, a sample identifies as being derived from an individual suffering from IA, or at risk of suffering from IA when the level of expression or enzymatic activity is altered in the indicative direction given in table A, B, 3, 4 and/or C by at least a factor of 1,5. Thus a gene is said to be deregulated in the context of the invention when the expression level or enzymatic activity of a gene product of said gene is altered in the indicative direction as given in tables A, B, 3, 4 and/or C by a factor of at least 1,5. When expression levels or enzymatic activity of gene products of more than one gene of table A, table B, table 3 or table 4 are determined, the accuracy of the diagnostic method increases allowing comparison of patterns of expression levels in the sample with patterns of a reference. In these cases, samples are identified as being derived from an individual suffering from IA, or at risk of suffering from IA when the pattern of expression levels or enzymatic activity matches the pattern of a reference derived from an individual suffering from IA, or at risk of suffering from IA. Or alternatively, identifies as normal, when the pattern matches the pattern of a reference. A reference as used in the invention is preferably from an arthritic sample, however, a normal sample can also be used. Preferably the reference is derived from at least an arthritic sample and one from a normal sample. Statistical methods to analyze patterns of expression levels are available in the art. A non-limiting statistical method for determining similarity of expression profiles, that can be used advantageously in the present invention is referred to as the Rosetta Error Model (Hughes T R, Marton M J, Jones A R et al. Functional discovery via a compendium of expression profiles. Cell. 2000; 102:109-126). A practical case is described on page 6 line 3-page 7 line 19 of WO 02/103320.

It is of course possible to enlarge the set of genes from which the expression level or enzymatic activity of gene products is determined. In a preferred embodiment, expression levels of gene products of at least 2 and more preferably at least 5 genes of table A, table B, or table 4 are analyzed. Analyzing said 5 genes provides a good correlation with the absence of presence of IA. Increasing the number of analyzed genes of table A or table B, further increases the correlation. Thus in a particularly preferred embodiment gene products or enzymatic activity of at least 10 genes of table A, B, or 4 are analyzed. In a preferred embodiment at least one gene product or enzymatic activity is of a gene of table A and at least one other gene product or enzymatic activity is of a gene of table B or C. In a particularly preferred embodiment said one or more, said at least 2, said at least 5 or said at least 10 genes are genes of table A, optionally supplemented by gene products or enzymatic activity of one or more genes of table B or table C. As mentioned, the set of genes analyzed may be expanded to other genes, preferably genes known to be deregulated in IA. Thus in a preferred embodiment said analysis includes the analysis of gene products of at least one gene of table C. The direction in which expression is affected in the arthritic samples is indicated in tables A, B and C(SF UP, SF down, WJ up, WJ down, where SF means that expression as indicated for synovial fibroblast cells and WJ means that expression is indicated for whole joint cells.

Genes can have several different gene products. Typically, though not necessarily, a gene is transcribed in mRNA, which in turn is translated into protein. A gene may be transcribed in several mRNAs which in turn can be translated into several proteins. As used herein, the term “gene product” encompasses both RNA and protein products. In a preferred embodiment a gene product in a method of the invention comprises RNA. Expression levels of RNA are typically determined with a probe that specifically hybridises with said RNA (Northern blot) and/or with a set of primers that specifically anneal to said RNA (Real Time RT-PCR). Expression levels of proteins are typically determined either directly with antibodies that specifically bind to the protein encoded by the gene or indirectly by the enzymatic/catalytically activity of the protein of interest. Many different kinds of probes are presently known in the art. Probes are often nucleic acids, however, alternatives having the same binding specificity in kind, not necessarily in amount are available to the person skilled in the art, such alternatives include but are not limited to peptide nucleic acids (PNA). Similarly, many different specific binding bodies are available. Of old, antibodies are used. However, currently many different parts, derivatives and/or analogues of antibodies are in use. Non-limiting examples of such parts, derivatives and/or analogues are, single chain Fv-fragments, monobodies, VHH, Fab-fragments and the like. A common denominator of such specific binding bodies is the presence of an affinity region (a binding peptide) that is present on a structural body that provides the correct structure for presenting the binding peptide. Binding peptides are typically derived from or similar to CDR sequences (typically CDR3 sequences) of antibodies, whereas the structure providing body is typically derived from or similar to framework regions of antibodies. For review of suitable probes and specific binding bodies see any of the following references:

Diagnosing early-onset rheumatoid arthritis: the role of anti-CCP antibodies, Vasishta A, Am Clin Lab. 2002 August-September; 21(7):34-6; Keys to unlocking the mysteries of rheumatic autoimmune disease, Moser K L, Gaffney P, Peterson E, Behrens T, Minn Med. 2004 May; 87(5):46-51; A new tool for rheumatology: large-scale analysis of gene expression Lequerre T, Coulouarn C, Derambure C, Lefebvre G, Vittecoq O, Daveau M, Salier J P, Le Loet X, Joint Bone Spine, 2003 August; 70(4):248-56.

Expression levels of gene products can be altered as result of a variety of mechanisms. These include but are not limited to, altered level of transcription/translation, altered stability, altered degradation of the protein and/or RNA. The altered expression level can also be the result of a combination of processes in a cell. The sample to be analyzed is typically derived from an individual suspected of suffering from IA or suspected of being at risk of suffering from IA. The sample can be either synovial membrane or synovial fluid isolated during an arthroscopy, and/or serum isolated from the blood of said individual.

An individual is typically a human although the expression level or enzymatic activity of the genes of interest could be determined in animal models to determine drug efficacy. Therefore an individual is preferably a mammal, preferably a rodent or a primate. More preferably a human.

A particular class of deregulated genes in the present invention is involved with cytoskeleton arrangement. The invention therefore in one aspect provides a method for determining whether a sample is derived from an individual suffering from inflammatory arthritis, preferably rheumatoid arthritis, or from an individual at risk of suffering thereof, said method comprising measuring in said sample the level of expression of at least one gene product of a gene involved in cytoskeleton arrangement and comparing said expression level with a reference value.

Cytoskeleton arrangement in a cell comprises both the structural processes involved with assembly and organisation of cytoskeleton and the signaling machinery in a cell that directs the structural processes. Assembly includes both gain and loss of cytoskeleton. Organisation typically includes the apparent three dimensional appearance of the cytoskeleton in a cell. The signaling machinery involved in cytoskeleton organisation has an extracellular component and an intracellular component, thus in a preferred embodiment said gene is involved in upstream/downstream extracellular or intracellular signaling to the cytoskeleton. A preferred embodiment of the invention is particularly related to deregulation of genes involved in assembly of actin cytoskeleton. Thus in a preferred embodiment, said gene is involved in binding and/or rearrangement of actin cytoskeleton. In a preferred embodiment said gene is involved in cytoskeleton arrangement in a synovial fibroblast. In a particularly preferred embodiment said gene is any one of the following: tubulin alpha 1, RAB14, Gelsolin, phosphatidylinositol membrane-associated, matrix metalloproteinase 3, matrix metalloproteinase 13, tissue inhibitor of matrix metalloproteinase 13, cathepsin S, matrix gamma-carboxylglutamate, integrin alpha X, myosin light chain, troponin T1 skeletal slow, troponin I skeletal fast 2, VASP, Gesolin and/or table 4. The individual is suffering from inflammatory arthritis when there is at least a differences of at least 1.5 in the level of expression of said gene product of a gene involved in cytoskeleton arrangement in said sample and said reference value. Preferably, said method comprises measuring in said sample the level of expression of a gene product of at least two of said genes involved in cytoskeleton arrangement and comparing said expression levels with reference values. In a particularly preferred embodiment the level of expression of a gene product of at least five of said genes involved in cytoskeleton arrangement is measured and compared with a reference value.

While measuring at least one gene products expression level or enzymatic activity and comparing it with a reference value, according to a method of the invention, one can determine a magnitude of a difference between said expression level and said reference level. In one embodiment a method according to the invention is provided, wherein a magnitude of a difference between said expression level and said reference value is normative for a degree in which said inflammation arthritis is present. A magnitude of a difference as used herein comprises at least a significant difference between the measured value and the reference value. A magnitude of difference can for example be determined by measuring fluorescence intensities. A difference between said expression level and said reference value is normative in that it ascertains a degree in which inflammatory arthritis is present. A degree in which inflammatory arthritis is present can either be a stage of IA that said disease has reached in an individual and/or it can be a measurement of severity of IA in an individual.

Deregulation of genes is a process that occurs in many different diseases. Each of those diseases has a characteristic gene or multiple genes that are deregulated in said diseases. IA is such a disease. IA is a generic term, which includes specific types of arthritis. Specific types of arthritis are typically associated with different characteristic genes that are deregulated. Deregulation of a gene will result in alteration of an expression level of a gene product of a gene and thus in an altered amount of a gene product or in alteration of a composition of a gene product. In one embodiment the invention provides the use of an expression level of a gene product of a gene of table A, table B, or table 4 for typing inflammatory arthritis in an individual. In a preferred embodiment expression levels of gene products of at least 2 and more preferably at least 5 genes of table A, table B, or table 4 are analyzed. Analyzing said 5 genes gives a good picture of the involvement of characteristic genes of a type of IA. Increasing the number of analyzed genes of table A, table B, or table 4, further increases the certainty about the type of IA concerned. Thus in a particularly preferred embodiment gene products of at least 10 genes of table A, table B, or table 4 are analyzed. In another preferred embodiment a gene product of a gene of table A is analyzed. In a further preferred embodiment expression levels of gene products of at least 2 and more preferably at least 5 genes of table A are analyzed. In a particularly preferred embodiment gene products of at least 10 genes of table A are analyzed. A sample is typed as derived from an individual or non-human animal suffering from or at risk of suffering from IA when the expression level of the tested genes is changed in the direction as indicated in tables A, B or C(SF UP, for genes that are over-expressed in synovial fibroblast cells in IA and SF-down for genes that are under-expressed in said cells in IA, WJ UP, for genes that are over-expressed in whole joint cells in IA and WJ down for genes that are under-expressed in whole joint cells in IA).

Inflammatory arthritis includes a number of specific arthritis conditions such as but not limited to Rheumatoid Arthritis, Systemic Lupus Erythematosus, Juvenile Rheumatoid Arthritis, Relapsing Polychondritis, Spondyloarthropathie, Ankylosing Spondylitis, Reiter's Syndrome, Psoriatic Arthritis, Enteropathic Arthritis, Crystal Deposition Disease, Gout Chondrocalcinosis, Calcium Hydroxyaptite Crystal Deposition Disease. In a preferred embodiment the invention is concerned with Rheumatoid Arthritis (RA). Thus when the invention refers to inflammatory arthritis in general, the preferred application is in rheumatoid arthritis.

Typing IA according to the invention preferably comprises determining a pattern of expression levels of gene products (i.e. higher than normal SF UP, WJ up or lower than normal SF down, WJ down in tables A, B and C), as a pattern of measured values is more specific than individual measured values. The invention thus provides a use according to the invention, wherein said typing comprises determining a pattern of expression levels of gene products of at least two genes in a sample and wherein at least one of said genes is a gene of table A, table B, or table 4. In a preferred embodiment at least one gene product is of a gene of table A and at least one other gene product is of a gene of table B or C. In a particularly preferred embodiment said one or more, said at least 2, said at least 5 or said at least 10 genes are genes of table A, optionally supplemented by gene products of one or more genes of table B or table C. As mentioned, the set of genes analyzed may be expanded to other genes, preferably genes known to be deregulated in IA. Thus in a preferred embodiment said analysis includes the analysis of gene products of at least one gene of table C.

In one embodiment the invention provides a use according to the invention, wherein said typing comprises indicating one of the following types: Rheumatoid Arthritis, Systemic Lupus Erythematosus, Juvenile Rheumatoid Arthritis, Relapsing Polychondritis, Spondyloarthropathie, Ankylosing Spondylitis, Reiter's Syndrome, Psoriatic Arthritis, Enteropathic Arthritis, Crystal Deposition Disease, Gout Chondrocalcinosis, Calcium Hydroxyaptite Crystal Deposition Disease. In a particularly preferred embodiment of the invention said typing comprises indicating an inflammatory arthritide. Said types of arthritis are all included in the generic term Inflammatory Arthritis as used in the invention.

In one embodiment of the invention genes and/or the expression level of gene products encoded thereby are detected. The expression level of their products or their enzymatic activity are determined with a probe or a set of probes or appropriate enzymatic assays. The invention thus provides a kit of parts comprising a probe or a set of probes or enzymatic assays, wherein at least one of said probes is a probe specific for one of the genes listed in table A, table B, or table 4. Preferably a set of probes comprises two or more probes that are specific for one of the genes listed in table A, table B, or table 4. In another embodiment the invention provides a kit of parts according to the invention, comprising a probe or a set of probes, wherein at least one of said probes is a probe specific for one of the genes listed in table A. The invention further provides a kit of parts according to the invention, comprising at least two probes, wherein at least one of said probes is a probe specific for one of the genes listed in table A, table B, or table 4 and at least one of said probes is specific for one of the genes listed in table C. Also provided by the invention is a kit of parts according to the invention, comprising at least two probes, wherein at least one of said probes is a probe specific for one of the genes listed in table A and at least one of said probes is specific for one of the genes listed in table B or in table C. Any of the probe sets according to the invention can comprise probes specific for a gene that is not listed in table A, B or C. Such a probe is preferably specific for a gene that is known to be involved in inflammatory arthritis, more preferably known to be involved in Rheumatoid Arthritis. In another preferred embodiment such a probe is specific for a gene known to be involved in cytoskeleton arrangement.

The present invention has surprisingly found that in IA particular genes involved with cytoskeleton arrangement are deregulated. The invention thereby presents a novel means of therapy. In one embodiment the invention hereto provides the use of a compound for altering cytoskeleton or its organization in a cell for the manufacture of a medicament for alleviating at least one symptom of inflammatory arthritis in an individual. An individual with IA can present either one symptom or multiple symptoms. Alleviating at least one symptom will make the condition of an individual with IA better, thereby improving a patient's quality of life. When reference is made to alleviating a symptom of a disease, it is meant that the severity of a symptom is at least reduced.

Rheumatoid arthritis is a polygenic disease. Studying multiple genes therefore provides more valuable information about RA than concentrating on one gene. Thus, in the invention concerted gene functions and cellular processes were studied in order to gather valuable clues and to prioritize targets for therapeutic intervention of RA. In this context, deregulated processes were determined as these are formalized through the controlled vocabulary of the Gene Ontology Consortium [17]. GO term frequencies were calculated in the selected gene list and their statistical significance was estimated. The resulting table (Supplementary Table 3) includes a number of functions that encompass accumulated knowledge about the pathogenesis of the disease, such as collagen catabolism, complement activation, immune and stress response. More importantly, 5 out of 26 predicted deregulated GO functions concerned (directly or indirectly) the actin cytoskeleton. In order to increase the experimental evidence F-actin stress fibers were visualized in vitro on arthritic as well as wt SFs, both primary and immortalized. As evident in FIG. 3a, there was a striking difference between arthritic and normal SF's, with the arthritic SFs exhibiting pronounced stress fibers. Stress fibers are known to appear in differentiated fibroblasts. These differentiated fibroblasts are called myofibroblasts. These myofibroblasts are specialized contractile fibroblasts, with an important role in establishing tension during wound healing and pathological contracture [21,22]. The invention hereby provides the first direct indication about the presence of myofibroblasts in the arthritic synovium. Consistent with this notion, arthritic SFs were observed to have more pronounced FAK positive islands (data not shown). FAK positive islands are a prominent feature of myofibroblasts [22]. The actin cytoskeleton interacts bi-directionally with the ECM through receptors (mainly integrins) that possess extracellular binding sites for laminin, collagen, fibronectin and other ECM components. The formation of intimate, extensive adhesive contacts between cells and ECM results from cooperation between adhesive systems and the actin cytoskeleton and the generation of force across regions of the cell [24]. In this context, myofibroblasts are thought to exert increased tension to the substratum, through their increased adhesive capacity, which results to a more flattened, elongated cell shape [22]. Accordingly, we have observed that arthritic SFs adhere to various ECM components with increased affinity in vitro (FIG. 3b), resulting in a more elongated shape in vivo (FIG. 3c), further corroborating the existence of myofibroblasts in the arthritic synovium.

The invention thus surprisingly found that deregulation of genes involved in cytoskeleton arrangement in a cell is a prominent feature in arthritic SF's. A new therapeutic and diagnostic entrance is hereby established. In one embodiment the invention provides the use of a compound for altering cytoskeleton or its organization in a cell for reducing stress fibers in a synovial cell.

Recent experiments have shown that the cytoskeleton plays a critical role in tumorigenesis [31]. Since the role of the cytoskeleton in cancer has been established, compounds that are directed to the cytoskeleton have been developed for the treatment of cancer. In one embodiment the invention provides the use of a drug for the treatment of cancer for altering cytoskeleton or its organization in a cell for the manufacture of a medicament for alleviating at least one symptom of inflammatory arthritis in an individual. In another embodiment the invention provides the use of a drug for the treatment of cancer for altering cytoskeleton or its organization in a cell for reducing stress fibers in a synvovial cell or for the treatment of inflammatory arthritis. Non-limiting examples of drugs that are directed to the cytoskeleton are: Cytochalasin D, Phalloidin, Jasplakinolide, Chondramides, Dolostatin 11, Latrunculin A, Swinholide A, Misakinolide A, Mycalolide B, Shinxolide C, Scytophycins, Goniodomin A, Pseudopterolide, Lysophosphatidylcholine, Lysophosphatidyl acid. In one embodiment the invention provides the use of a compound according to the invention, wherein said compound is one of the following: Cytochalasin D, Phalloidin, Jasplakinolide, Chondramides, Dolostatin 11, Latrunculin A, Swinholide A, Misakinolide A, Mycalolide B, Shinxolide C, Scytophycins, Goniodomin A, Pseudopterolide, Lysophosphatidylcholine, Lysophosphatidyl acid.

Many different animal models are used in studies of RA. An animal model typically represents a patient with RA. In order to make an animal represent an individual suffering from RA different strategic routes can be applied. Disease can for example be induced by injection of an immunogenic substance. Alternatively an animal can be bred that has an altered gene expression. A gene in such an animal can either be knocked-out, over-expressed or under-expressed. The invention provides an animal with an altered gene expression. In one embodiment the invention provides a non-human animal wherein at least one of the genes listed in table A, table B, table 3 or table 4 is knocked-out, over-expressed or under-expressed. In another embodiment the invention provides a non-human animal wherein at least one of the genes listed in table A is knocked-out, over-expressed or under-expressed. The invention further provides a non-human animal wherein at least one of the genes listed in table A, table B, table 3 or table 4 and wherein at least one of the genes listed in table C or table 6 is knocked-out, over-expressed or under-expressed. Furthermore the invention provides a non-human animal wherein at least one of the genes listed in table A and wherein at least one of the genes listed in table B, table C, table 3, table 4 or table 6 is knocked-out, over-expressed or under-expressed. Naturally the invention also provides an animal wherein, besides a gene listed in table A, B, 3, 4, 6 or C, another gene is knocked-out, over-expressed or under-expressed. Said another gene preferably is a gene known to be involved in RA. Another preferred gene in this context is a gene known to be involved in cytoskeleton rearrangement. The invention in one embodiment provides a non-human animal wherein at least one gene known to be involved in cytoskeleton rearrangement is knocked-out, over-expressed or under-expressed.

In RA one of the cell types that plays a very important role in the disease process is the synovial fibroblast. Therefore, in a preferred embodiment, the invention provides a non-human animal according to the invention, wherein said cytoskeleton arrangement is in a synovial fibroblast. Cytoskeleton organization is typically influenced by diverse signals. Therefore the invention in one embodiment provides a non-human animal according to the invention, wherein said gene is involved in extracellular or intracellular signaling to the cytoskeleton. In a preferred embodiment the invention provides a non-human animal according to the invention, wherein said gene is involved in binding and/or rearrangement of the actin cytoskeleton. The invention provides genes that are involved in cytoskeleton arrangement. The invention thus in a preferred embodiment provides a non-human animal according to the invention, wherein said gene is any one of the following: tubulin alpha 1, RAB14, Gelsolin, phosphatidylinositol membrane-associated, matrix metalloproteinase 3, matrix metalloproteinase 13, tissue inhibitor of matrix metalloproteinase 13, cathepsin S, matrix gamma-carboxylglutamate, integrin alpha X, myosin light chain, troponin T1 skeletal slow, troponin I skeletal fast 2, VASP, ADF/Cofilin and/or is gene of table 4. Preferably said gene is Gelsolin, VASP and or ADF/Cofilin. These genes are further discussed in the examples, tables and figures.

An alternative means to create an animal wherein a gene is over-expressed or under-expressed is the introduction of a foreign gene into the genome of said animal. Such an animal is a transgenic animal. The invention in one embodiment provides a non-human animal according to the invention, wherein said animal is transgenic for said gene or homologue thereof that is over-expressed or under-expressed. A foreign gene can be constructed using recombinant DNA methodology. A preferred non-human animal as provided by the invention is rodent or a non-human primate. Preferably, said non-human animal is a mouse. Characteristics of these animals are well known and these animals are practical for laboratory circumstances. Mice and rats are often used in disease models for RA. In a preferred embodiment the invention provides the use of a non-human animal according to the invention as a disease model. Preferably said disease represented by said model is rheumatoid arthritis. Alternatively said disease is a disease in which deregulated genes as determined by the invention are involved. Inflammatory and/or rheumatoid arthritis in an animal can either occur spontaneously, by known or unknown internal causes, or can be induced. In a preferred embodiment the invention provides the use of a non-human animal as a disease model for rheumatoid arthritis, wherein said rheumatoid arthritis is induced. Induction can proceed directly or indirectly. Direct induction for example occurs when an immunogenic composition that causes arthritis is injected. Examples of such immunogenic compositions include but are not limited to collagen. Indirect induction is for example due to a genetic defect, because of which an animal at a certain stage of its life develops arthritis. Preferred examples of such induction are indicated in table 5.

Further preferred is a non-human animal of the invention wherein said at least one of the genes listed in table A, table B, table C, table 3, table 4 or table 6 is knocked-out, over-expressed or under-expressed, in an arthritis model. Preferably a genetic arthritis model, preferably a genetic model as indicated in table 5, and-or in a Tg197 mouse. Provided is preferably a Tg197 mouse wherein Gelsolin, VASP and/or ADF/Cofilin is knocked-out, over-expressed or under-expressed. Preferably specifically in the myeloid lineage. Preferably, Gelsolin and/or ADF/Cofilin are knocked out or under-expressed. VASP is preferably over-expressed.

The invention further provides the use of a non-human animal of the invention as an inflammatory arthritis model. Preferably, said non-human animal is suffering from inflammatory arthritis. This is typically achieved by providing a non-human animal of the invention with a composition that induces said inflammatory arthritis. Such compositions are well known in the art and include but are not limited to collagen (are there other well known examples).

Inflammatory inflammation being a severe disease for which no definite cure has been found, there is an ongoing need for effective medication for diverse types and stages of RA. In the process of searching for effective medication a reliable test for compounds comprised in this medication is essential. The invention provides a method for determining whether a compound is effective in alleviating a symptom of IA, comprising testing said compound in a model system for said symptom, said method characterized in that said compound is a compound for altering the cytoskeleton or its organization in a cell. As in the development of cancer therapy compounds have been found that are involved in cytoskeleton arrangement, said compounds are used in a method of the invention. Thus, in one embodiment the invention provides a method for determining whether a compound is effective in alleviating a symptom of IA, comprising testing said compound in a model system for said symptom, said method characterized in that said compound is a compound for altering the cytoskeleton or its organization in a cell, wherein said compound is effective in cancer therapy. Said compound preferably is one of the following: Cytochalasin D, Phalloidin, Jasplakinolide, Chondramides, Dolostatin 11, Latrunculin A, Swinholide A, Misakinolide A, Mycalolide B, Shinxolide C, Scytophycins, Goniodomin A, Pseudopterolide, Lysophosphatidylcholine, Lysophosphatidyl acid.

In the invention specific genes have been found to be deregulated in IA. In the search process for compounds with therapeutic value for treatment of IA, the expression of these genes is a useful parameter. For other diseases in which the deregulated genes that are found in the invention are deregulated, said parameter can be used as well. The similar deregulation for instance occurs because of a similar causal mechanism that acts alike in these diseases. The invention provides a method for determining whether a compound has an effect on an expression of one or more of the genes listed in table A, table B, or table 4, comprising testing said compound in an environment wherein said gene is expressed and comparing a resulting expression level with a reference value. Preferably reference value indicating a higher than normal expression level for the genes as listed in table A, B or C by SF UP and/or WJ up or a lower than normal expression level as indicated in table A, B or C by SF down, and/or WJ down. The invention further provides a method for determining whether a compound has an effect on an expression of one or more of the genes listed in table A, comprising testing said compound in an environment wherein said gene is expressed and comparing a resulting expression level with a reference value. In one embodiment the invention provides a method for determining whether a compound has an effect on an expression of one or more of the genes listed in table A, table B, or table 4 and on one or more of the genes listed in table C, comprising testing said compound in an environment wherein said genes are expressed and comparing a resulting expression level with a reference value. In another embodiment the invention provides a method for determining whether a compound has an effect on an expression of one or more of the genes listed in table A and on one or more of the genes listed in table B or in table C, comprising testing said compound in an environment wherein said genes are expressed and comparing a resulting expression level with a reference value. Preferably an effect on multiple genes that are listed in table A, B or C is determined.

Genes that have been found to be deregulated in the invention are in one embodiment of the invention used in a disease model of IA. In such an embodiment said genes are deregulated on purpose. In contrast, in an individual with IA, said genes already are deregulated. In this context, the purpose is to regulate said genes in order to improve the condition of said individual. In one embodiment the invention therefore provides a method for alleviating at least one symptom of rheumatoid arthritis in an individual, comprising modifying expression in said individual of a gene that is involved in a cytoskeleton arrangement. Modifying expression as used herein alters the expression of said deregulated genes in said individual in such a way that the condition of said individual is improved. Alleviating a symptom of IA in a patient will usually imply up-regulating a down-regulated gene or down-regulating an up-regulated gene (see table A, B or C SF or WJ down and SF or WJ UP respectively), such that the expression of said gene approaches or equals a reference value of a normal, i.e. healthy, state. In a preferred embodiment the invention provides a method for alleviating at least one symptom of rheumatoid arthritis in an individual, comprising modifying expression in said individual of a gene that is involved in a cytoskeleton arrangement, wherein expression of said gene is modified in a synovial fibroblast. In one embodiment of the invention said gene is involved in extracellular or intracellular signaling to the cytoskeleton. In another embodiment said gene is involved in binding and/or rearrangement of the actin cytoskeleton. The invention discloses genes that are involved in cytoskeleton arrangement and deregulated in IA. In one embodiment the invention thus provides a method according to the invention, wherein said gene is any one of the following: tubulin alpha 1, RAB14, Gelsolin, phosphatidylinositol membrane-associated, matrix metalloproteinase 3, matrix metalloproteinase 13, tissue inhibitor of matrix metalloproteinase 13, cathepsin S, matrix gamma-carboxylglutamate, integrin alpha X, myosin light chain, troponin T1 skeletal slow, troponin I skeletal fast 2, VASP, ADF/Cofilin and/or a gene of table 4. A method of the invention is also applicable in another disorder than IA wherein genes involved in cytoskeleton arrangement, such as aforementioned genes, are deregulated. In a preferred embodiment said gene in a method according to the invention codes for Gelsolin, VASP or ADF/Cofilin. Preferably Gelsolin.

Gelsolin is an actin binding protein [33] that has been implicated, amongst others, in the transduction of signals into dynamic rearrangements of the cytoskeletal architecture. In the presence of calcium, gelsolin severs preexisting actin filaments and caps them, thereby preventing monomer addition to their fast-growing ends. The barbed end cap is highly stable, even in the absence of calcium, unless displaced by interactions with regulatory phospholipids such as phosphatidylinositol-4,5-bisphosphate (PIP2). In the presence of a large pool of profilin (another actin binding protein), or under depolymerizing conditions, these gelsolin-capped ends allow the disassembly of populations of actin filaments by subunit loss from the pointed ends [34]. Gsn−/− fibroblasts were found to have excessive stress fibers in vitro [18], very similar to the ones observed in arthritic SFs, where gelsolin was found down-regulated with a variety of methods. Knocking out gsn expression from the arthritic mice resulted in exacerbation of the disease (FIG. 4), therefore proving the participation of the actin cytoskeleton rearrangement in the pathophysiology of the disease. Gelsolin was found to be one of the most striking downregulated markers upon malignant transformation of fibroblasts by Ras [35], while overexpression of a gelsolin mutant was shown to suppress Ras-induced transformation [36]. Its expression was found undetectable or reduced in a majority of human gastric, bladder, lung, colon and breast tumours [37-39].

The genes that are involved in cytoskeleton arrangement are only one group of genes that, in the invention, are found to be deregulated in IA. Other genes are, amongst others, listed in table A, table B, table 3, table 4 or C. The invention in one embodiment provides a method for alleviating at least one symptom of inflammatory and/or rheumatoid arthritis in an individual, comprising modifying the level of expression of at least one gene product of a gene of table A, table B, table 3, table 4 or C in cells of said individual. In another embodiment said method comprises modifying the level of expression of at least one gene product of a gene of table A in cells of said individual. In a further embodiment said method comprises modifying the level of expression of at least one gene product of a gene of table A, table B, or table 4 and at least one gene product of a gene of table C in cells of said individual. Further the invention provides a method according to the invention, wherein said method comprises modifying the level of expression of at least one gene product of a gene of table A and at least one gene product of a gene of table B or table C in cells of said individual. In preferred circumstances a method according to the invention alleviates two or more symptoms. Preferably the level of expression of two, three or more gene products of genes of table A, B, C, or 4 are modified. The direction in which an expression level is modified is dependent on the direction in which an expression level of a gene product of a gene is deregulated (see table A, B or C). The extent to which modification is applied depends on the degree in which said level of expression of said gene product of said gene is deregulated. The expression level of a gene product of a gene is preferably modified to match or approach a reference value that represents a healthy status, thus a normal level. Alternatively, the expression level of a gene product of a gene is modified to match a physiologically acceptable level of an individual. Physiologically acceptable levels will often be levels that are approximately normal levels. Normal levels are levels that can be found in a healthy individual of the same age and constitution. Physiologically acceptable levels can fall outside a normal range but do still provide a sufficient function for an individual.

A gene product in a method of the invention encompasses both RNA and protein products. In one embodiment the invention provides a method for alleviating at least one symptom of inflammatory and/or rheumatoid arthritis in an individual according to the invention, wherein said gene product comprises protein. A cell in which the level of expression of a gene product of a gene is modified in a method of the invention, is a cell that is affected by the disease process of inflammatory and/or rheumatoid arthritis in an individual. Preferably said cell is a cell that makes contact with a joint of said individual. In a preferred embodiment of the invention, a method according to the invention is provided, wherein said cells are cells of a joint or precursors thereof. These cells can be part of any component of the joint. Precursor cells can already be present in or in the surroundings of said joint or they can for example be derived from the blood. In a preferred embodiment said cells comprise synovial membrane cells or cells of synovial fluid. Preferably in a method according to the invention the level of expression of at least two gene products is modified. In a preferred embodiment a method according to the invention comprises modifying the level of expression of at least one gene product of a gene of table A, table B, or table 4 in cells of said individual and modifying the level of expression of at least one gene product of a gene known to be deregulated in IA other than listed in table A, B or C. Further provided is a method according to the invention, wherein said method comprises modifying the level of expression of at least one gene product of a gene of table A in cells of said individual and modifying the level of expression of at least one gene product of a gene known to be deregulated in IA other than listed in table A, B or C. In a further embodiment said method comprises modifying the level of expression of at least one gene product of a gene of table A or table B and at least one gene product of a gene of table C in cells of said individual and modifying the level of expression of at least one gene product of a gene known to be deregulated in IA other than listed in table A, B or C. Further the invention provides a method according to the invention, wherein said method comprises modifying the level of expression of at least one gene product of a gene of table A and at least one gene product of a gene of table B or table C in cells of said individual and at least one gene product of a gene known to be deregulated in IA other than listed in table A, B or C.

In a preferred embodiment, the invention provides a method for alleviating at least one symptom of inflammatory and/or rheumatoid arthritis in an individual according to the invention, wherein modifying said expression level comprises lowering expression of a gene that is over-expressed in said individual compared to a comparable healthy individual, or elevating expression of a gene that is under-expressed in said individual compared to a comparable healthy individual (table A, B or C).

Raising expression of an under-expressed gene is for instance effected by the addition of a transcription factor and/or by activating an enhancer-sequence. Lowering expression of an over-expressed gene can for example be realized by applying antisense therapy or by administering a repressor protein that binds to the promoter of said aberrantly expressed gene. In a preferred embodiment, the invention provides a method according to the invention, wherein said lowering said expression comprises utilizing an antisense-sequence. There are many different anti-sense approaches to down-regulate production of a gene product available to a person skilled in the art. Antisense technology exploits oligonucleotide analogs to bind to target RNAs via Watson-Crick hybridization. After binding, the antisense agent either disables or induces the degradation of the target RNA. Antisense agents can also alter splicing. The basic mechanisms of antisense technology have been extensively explored in the last ten years. Much has been learned about the medicinal chemistry and the pharmacologic, pharmacokinetic, and toxicologic properties of antisense molecules. Antisense technology has proven valuable in gene functionalization and target validation.

Non-limiting examples of antisense approaches are interference RNA (RNAi), microRNA and splice interference techniques such as exon-skipping. An antisense sequence can be administered as a single stranded molecule or be part of a hairpin construct-molecule. The anti-sense sequence can be administered directly or be produced in a cell by means of an expression cassette. Today RNA interference (RNAi), is a technique in which exogenous, double-stranded RNAs (dsRNAs) that are complimentary to known mRNA's, are introduced into a cell to specifically destroy that particular mRNA, thereby diminishing or abolishing gene expression. The technique has proven effective in Drosophila, Caenorhabditis elegans, plants, and recently, in mammalian cell cultures. For research purposes in cultured mammalian cells, small interfering RNAs (siRNAs) are delivered into the cell. SiRNAs are dsRNAs of approximately 21-25 nucleotides. This is done with transfection reagents, solutions optimized for allowing DNA and RNA to be absorbed by cultured cells. In a preferred embodiment the invention provides a method according to the invention, wherein lowering said expression comprises utilizing RNAi. Preferably siRNA is used for the induction of RNAi. In another embodiment shRNA is used. A classical antisense sequence can be complementary to at least 10% and preferably at least 25 of the mRNA it is specific for. SiRNA, miRNA, RNAi and exon-skipping antisense sequence have the normal overlap that is typically used in the field. They typically have between 15-40 bases overlap, preferably between 15-30. SiRNAs have typically 21-25 nucleotides. The antisense can be provided as single-stranded or double stranded molecules, depending on their effectivity as is commonly known in the art. The antisense sequence can be comprised in a nucleic acid or a functional equivalent thereof.

An anti-sense sequence can for example be used in a compound that is applied to modify an expression level of a gene product of a gene. In a preferred embodiment the invention provides the use of a compound for the manufacture of a medicament for alleviating at least one symptom of inflammatory and/or rheumatoid arthritis in an individual, said compound comprising an antisense-sequence of one of the genes listed in table A or table B. In another embodiment the invention provides a use according to the invention, wherein said compound comprises an antisense-sequence of one of the genes listed in table A. Further provided is a use according to the invention, wherein said compound comprises an antisense-sequence of one of the genes listed in table A or table B and an antisense-sequence of one of the genes listed in table C. In another use according to the invention, said compound comprises an antisense-sequence of one of the genes listed in table A and an antisense-sequence of one of the genes listed in table B or in table C. Preferably antisense-sequences of two or more genes listed in table A, B or C are comprised in a use of the invention. In addition to antisense-sequences of aforementioned genes, antisense-sequences of other genes known to be involved in IA can be comprised in said compound. In preferred embodiment an antisense is provided specific for mRNA from a gene that is identified in table A, B or C by SF UP or WJ up.

Before a suitable drug can be developed, a drug target has to be identified and validated. Drug targets are mainly proteins, DNA or RNA. The invention provides such drug targets as it has identified genes and gene products that are deregulated in IA. Thus in one embodiment the invention provides the use of a protein encoded by a gene of table A, table B, or table 4 as a drug target for selecting candidate drugs for a treatment of inflammatory arthritis. Further provided is a use according to the invention, wherein said protein is encoded by a gene of table A. In one embodiment a first protein encoded by a gene of table A or table B is used as a first drug target and a second protein encoded by a gene of table C is used as a second drug target. In another embodiment said first protein encoded by a gene of table A is used as a first drug target and a second protein encoded by a gene of table B or table C is used as a second drug target. By a use of the invention said drug targets are thus validated. After validation the drugs are tested further, first in a pre-clinical stage and then in a clinical setting.

As already mentioned, the invention provides probes specific for deregulated genes in IA. Probes thus provided, are also applicable for disorders other than IA in which said genes are deregulated. In order to make the addressing of a gene more specific, more then one probe is preferentially used. More then one probe, as used in the invention, is preferably comprised in an array. An array is used to analyze one or more samples at the same time for one or more genes or gene products. An array can be specific for a whole genome, but in the invention preferentially is specific for a part of a genome, i.e. a subgenome. Another preference of the invention is an array specific for a transcriptome or a part of a transcriptome, i.e. a subtranscriptome. A transcriptome of an organism as used herein, are all DNA transcripts, mostly mRNA's, being produced at a certain point. A certain point is specified in its location and/or in time. Thus in a preferred embodiment of the invention, an array is a subgenome array or a subtranscriptome array. In a preferred embodiment the invention provides an array comprising at least two probes, wherein at least one probe is specific for at least one gene listed in table A, table B, or table 4. The invention further provides an array according to the invention, wherein at least one probe is specific for at least one gene listed in table A. Also provided by the invention is an array according to the invention, wherein at least one probe is specific for at least one gene listed in table A or table B and wherein at least one probe is of at least one gene listed in table C. In another embodiment the invention provides an array according to the invention, wherein at least one probe is specific for at least one gene listed in table A, and wherein at least one probe is specific for at least one gene listed in table B or in table C. In a preferred embodiment an array of the invention is a microarray. Said probes specific for said genes are thus preferably comprised in a microarray. Said microarray preferably comprises oligonucleotides or a functional equivalent thereof. Probes or oligonucleotides are preferably between 15-80 nucleotides or functional equivalents thereof long. More preferably between 15-40.

An array as provided by the invention is in one embodiment used in a method of the invention. Provided is the use of an array according to the invention in a method according to the invention for determining whether a sample is derived from an individual suffering from inflammatory and/or rheumatoid arthritis or from an individual at risk of suffering there from, said method comprising measuring in said sample the level of expression of at least one gene product of a gene of table A or table B and comparing said expression level with a reference value. Further provided is the use of an array according to the invention for determining whether an individual is suffering from IA or is at risk of suffering from IA.

Protein expression levels can be measured in various ways such as ELISA, Western blotting or another sort of antibody based calorimetric or fluorescent quantitation of protein. In a preferred embodiment the invention provides an array comprising at least two binding bodies, wherein at least one binding body is specific for at least one gene product of a gene of table A, table B, or table 4. In on embodiment said binding body is an antibody. Said gene product is RNA or a protein. In one embodiment the invention provides an array according to the invention, wherein said gene product is a protein. In a preferred embodiment an array according to the invention comprises at least one binding body that is specific for at least one gene product of a gene of table A. Further provided is an array according to the invention, wherein at least one binding body is specific for at least one gene product of a gene of table A. In one embodiment the invention provides an array according to the invention, wherein at least one binding body is specific for at least one gene product of a gene of table A, table B, or table 4, and wherein at least one gene product is of a gene of table C. In another embodiment the invention provides an array according to the invention, wherein at least one binding body is specific for at least one gene product of a gene of table A, and wherein at least one gene product is of a gene of table B or table C. Said array according to the invention preferably is a microarray. Said array is in one embodiment used in a method according to the invention. Provided is the use of an array according to the invention in a method according to the invention for determining whether a sample is derived from an individual suffering from inflammatory and/or rheumatoid arthritis or from an individual at risk of suffering there from, said method comprising measuring in said sample the level of expression of at least one gene product of a gene of table A or table B and comparing said expression level with a reference value. Further provided is the use of an array according to the invention for determining whether an individual is suffering from IA or is at risk of suffering from IA.

Where in the description reference is made to table A or table B this reference also includes the reference to tables 3 and 4. In these cases a preference is indicated for tables A, B or table 3. Preferred is Table A or table B, Preferred is at least table A.

The invention further provides a method for determining whether a compound, a (poly)peptide or a nucleic acid molecule or functional equivalent thereof affects a phenotype of synovial fibroblast cells of an individual or non-human animal suffering from inflammatory arthritis or at risk of suffering thereof, comprising culturing synovial fibroblast cells in vitro from a sample obtained from an affected joint or a joint at risk of being affected from said individual or non-human animal, in the presence of said compound and/or providing said synovial fibroblast cells with said nucleic acid or said functional equivalent thereof and determining whether a phenotype of said cultured synovial fibroblast cells is altered. Preferably, wherein said synovial fibroblast cells are obtained from a joint of a mouse comprising the genetic alteration of the Tg197 mouse, a non-human animal according to the invention and/or a genetic model as depicted in table 5. In a preferred embodiment said compound, nucleic acid or functional equivalent thereof is a compound for altering cytoskeleton or its organization in a cell. Preferably said compound is effective in cancer therapy, as described herein above. Preferably said compound is one of the following:

Cytochalasin D, Phalloidin, Jasplakinolide, Chondramides, Dolostatin 11, Latrunculin A, Swinholide A, Misakinolide A, Mycalolide B, Shinxolide C, Scytophycins, Goniodomin A, Pseudopterolide, Lysophosphatidylcholine, Lysophosphatidyl acid. In a preferred embodiment said nucleic acid comprises an antisense-sequence, preferably an RNAi, an siRNA, or an shRNA. Said antisense sequence can be provided in a nucleic acid or a functional equivalent thereof. Said nucleic acid or functional equivalent thereof preferably comprises a morpholino, locked nucleic acid (LNA) or a peptide nucleic acid (PNA). In a particularly preferred embodiment said antisense-sequence is an antisense sequence of a gene listed in table A, B, C, or table 4, wherein said gene is identified in either of said tables by expression SF Up or WJ Up or said gene is VASP. In a particularly preferred embodiment said antisense sequence is specific for a gene of table A indicated in table A by expression SF UP or WJ up. It is preferably determined whether stress fiber formation, existence is affected. In another preferred embodiment said phenotype comprises adhesion, migration, proliferation, wound-healing and/or apoptosis of said synovial fibroblast cells. The present in vitro assay is particularly suited for high throughput screening, particularly for screening candidate drugs and/or nucleic acids of the invention for potential therapeutic effect in IA. In a preferred embodiment said nucleic acid codes for a protein produced by a gene of table A, B, C, or 4, wherein said gene is identified in table A, B or C by SF down and/or WJ down or said gene is gelsolin or ADF/Cofilin. An antisense sequence is preferably specific for a gene that is identified in table A, B or C by SF or WJ UP, or VASP. Said (poly)peptide is preferably an antibody or functional equivalent thereof such as but not limited to a single chain antibody, a monobody, a FAB fragment or the like. A peptide preferably comprises at least 6, and preferably at least 8 amino acids in peptide linkage. A peptide is preferably not longer than 40 amino acids. A polypeptide comprises 41 or more amino acids in peptidic linkage. A (poly)peptide may of the invention may comprise modifications such as chemical modifications and/or post translational modifications such as in a protein. A (poly)peptide may be linear or circular.

The present invention further provides the use of a gene delivery vehicle comprising the coding region of a gene of table A, B or C identified by SF and/or WJ down, Gelsolin or ADF/Cofilin, for delivering said gene to cells of a joint. Preferably said gene is delivered to a synovial fibroblast cell. In another aspect the invention provides the use of a gene delivery vehicle comprising the coding region of a gene of tables A, B or C identified by SF or WJ down, Gelsolin or Cofilin, for the preparation of a composition for delivering said gene to cells of a joint (WJ down) and/or a synovial fibroblast cell (SF down). Preferably said gene delivery vehicle is used for the treatment of inflammatory arthritis. In a particularly preferred embodiment said gene is Gelsolin, ADF/Cofilin. The invention thus further provides a gene delivery vehicle comprising a coding region for Gelsolin and/or ADF/Cofilin. Further provided is the use of a gene delivery vehicle comprising a nucleic acid of a functional equivalent thereof comprising an antisense-sequence for a gene of table A, B or C identified by SF and/or WJ UP or VASP, or comprising an expression cassette therefore, for delivering said nucleic acid or functional equivalent thereof to cells of a joint, or to a synovial fibroblast cell. Preferably wherein said antisense-sequence comprises an shRNA, RNAI and/or siRNA. Preferably wherein said gene delivery vehicle comprises a liposome, or a viral vector. Preferably an adenovirus, an adeno-associated virus vector or a lentivirus vector. The gene delivery vector may be delivered/administered systemically. In a particularly preferred embodiment said gene delivery vehicle is administered locally, preferably intrarticular. Said expression cassette preferably comprises a promoter to drive expression of the coding region and/or antisense sequence. Preferably said promoter is a pol II promoter or a pol III promoter.

### BRIEF DESCRIPTION OF THE DRAWINGS

FIG. 1. Chromosomal localization of identified deregulated genes in the RA animal model, together with the Quantitative Trait Loci (QTL) for Collagen Induced Arthritis (CIA). Red/Green arrows indicate up/down-regulated genes respectively. Black/Blue lettering refers to deregulated genes in whole joints/synovial fibroblasts respectively.

FIG. 2. Confirmation of deregulated expression in human patient samples. Quantitative RT-PCR for the indicated genes for 19 RA and 8 OA samples. Values were normalized to the expression of B2m and were expressed as expression index. Similar results were obtained upon normalization to L32. p-values were obtained in accordance to non parametric Mann-Whitney statistical test.

FIG. 3. Arthritic SFs have a rearranged cytoskeleton and increased ECM adhesion. a. Immunofluoresence of arthritic (RA) or normal (wt) primary and immortalized synovial fibroblasts (pSFs and iSFs respectively) for F-actin. b. Adhesion assays of arthritic or normal primary and immortalized SFs on purified ECM components. c. Transmission electron microscopy (TEM, magnification×5000) of synovial fibroblasts (SFs) from ankle joints isolated from wt and arthritic mice. N, nuclei; r-ER, rough endoplasmic reticulum; Arrow heads indicate dilation of r-ER, while arrows point to swollen mitochondria with distorted cristae.

FIG. 4. Knocking out gelsolin expression results in disease (RA) exacerbation. a. Histopathological scores. * p=0.025 n=8-11. b. Representative histopathological analysis (H/E) of arthritic joints. Shown images were assembled from multiple overlapping sections. Arrows indicate the synovial membrane.

FIG. 5. SQ-RT-PCR analysis of non-muscle cofilin in the Tg197 animal model.

FIG. 6. Q-RT-PCR analysis of mVASP in Tg197 versus wild-type whole joint and synovial fibroblasts.

FIG. 7. In vitro assays studying the properties of Tg SF's versus Wt SF's (A) and (B) Tg SFs exhibit increased adhesion and migration to fibronectin. Mean averages of triplicates with mean background (adhesion to BSA) values subtracted. Representative experiment out of three (t-test; P<0.05). (C) DNA synthesis/proliferation assay in the presence or absence of 10% FBS, 48 h after treatment. Values represent [3H]Thymidine incorporation (×10³). Mean average of triplicates (t test; P<0.05). (D) Assay of wound-healing treated with Mytomycin C for 2 h. Pictures were taken at Oh and 24 h after the wound (Oh not shown). (E) TNF cytotoxicity assay with cyclohexamide. After 24 h survival was determined using the crystal violet assay. (F) Apoptosis assay using 30-300 μM etoposide. (G) Apoptosis assay using 0.01-1 μM staurosporine. This figure shows that one of the properties of Tg-SF's is that they exhibit increased proliferation compare to Wt-SF's (in addition to increased Adhesion, Migration and Resistance to apoptosis).

FIG. 8. Adhesion/Migration assays showing the effect of Lat-A on Tg197 actin-cytoskeleton properties.

FIG. 9. Proliferation assays revealing the effect of Lat-A on apoptosis of Tg197 synovial fibroblasts (p<0.05, Student t test).

FIG. 10. Apoptosis assays revealing the effect of Lat-A on apoptosis of Tg197 synovial fibroblasts (p<0.05, Student t test).

FIG. 11. DEGs validation of expression on human patient samples. Q-RT-PCR analysis for the indicated genes for 12 OAs (osteoarthritis) and 21 RA samples. Values were normalized to the expression of (a) B2M, (b)L32 and expressed as expression index. Nonparametric Mann-Whitney statistical tests were used to derive p-values.

### EXAMPLES

### Example 1

### Materials and Methods

Animals. All transgenic mice were bred and maintained on a mixed CBAxC57BL/6 genetic background and kept at the animal facilities of the B.S.R.C. “Al. Fleming” under specific pathogen-free conditions, in compliance with the Declaration of Helsinki principles. (Temperature 20-22° C., Humidity 55±5%, 12 hours light/dark cycle, Water ad libitum). “Arthritic” transgenic mice (Tg197, hTNF^{+/-}), carried the human TNF gene where the 3′ UTR was replaced by the corresponding one from b-globin [5]. “Arthritic” knock in mice (TNF^{ΔARE/+} on 129/C57BL6) expressed the endogenous mTNF gene, where 69 bp encompassing the TNF ARE (AU-rich elements) at the 3′ UTR have been deleted, resulting in increased message stability and translational efficiency [8]. Overall, 86 mice were used for the experiments shown in this study (since some experiments were only representative ones), as listed explicitly where appropriate.

Cell isolation and culture. Synovial Fibroblasts (VCAM⁺) were isolated from 6-8 weeks old mice essentially as previously described [7,40]. Fibroblasts were selected by continuous culturing for at least 21 days and a minimum of four passages. No macrophage markers could be detected by FACS analysis. Cells were grown at 37° C., 5% CO₂ in complete DMEM medium (Gibco/BRL) supplemented with 10% FBS and 100 Units/ml of penicillin/streptomycin. The creation and culture conditions (33° C., 10 Units/ml of murine rIFN-γ) of conditionally immortalized synovial fibroblast cell lines and has been described previously [7].

RNA extraction. Total RNA was extracted from sub-confluent (70-80%) cultured Synovial Fibroblasts (primary or conditionally immortalized) with the RNAwiz reagent (Ambion Inc.), followed by single passage through an RNeasy column (QIAGEN, Hilden, Germany) according to manufacturer's instructions. Total RNA was extracted from whole joints using the Guanidinium isothiocynate/acid phenol protocol [41], followed by single passage through an RNeasy column. RNA integrity was assessed by electrophoresis on denaturing 1.2% agarose/formaldehyde gels. RNA quantity/quality was calculated based on OD readings at 260/280 nm.

Generation of full-length cDNA libraries. 1^{st} strand cDNA synthesis was performed using SUPERSCRIPT II (Life technologies/Invitrogen) at 56° C. in the presence of trehalose and sorbitol. The cap structure at the 5′ end of mRNAs was biotinylated and full length cDNAs were selected, after RNAse I treatment, using streptavidin-coated beads [42]. 2^{nd} strand synthesis was primed by the single-strand linker ligation method (SSLLM), where a double stranded DNA linker (with random 6-bp, dN6 or dGN5, 3′ overhangs) is ligated to the single-stranded cDNA [43]. The 2^{nd} strand is made by primer extension using mixtures of long-range thermostable polymerases, followed by restriction digestion (BamHI nd XhoI) and ligation to the λ-FLCI phagemid vector [43,44]. After packaging, cDNA libraries were amplified on solid phase, as previously described [45].

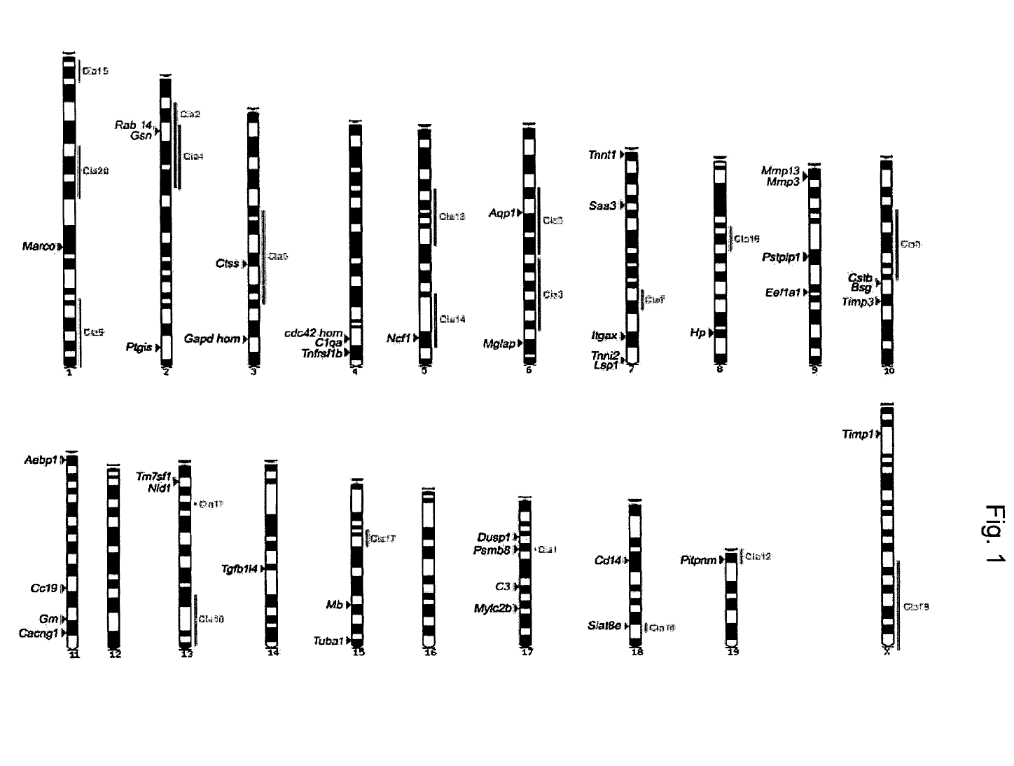

cDNA libraries normalization and subtraction (outlined in Supplementary FIG. 1a). Amplified, phagemid λ-FLCI cDNA libraries were used to infect the Cre-expressing bacterial cell line (BNN-132) and the excised plasmids were isolated [44]. Double stranded plasmid DNA was nicked by the site specific (f1 origin) endonuclease GeneII and converted to circular single stranded by digestion with Exonuclease III [45]. Circular, single stranded plasmid cDNA libraries (tester libraries) were then subjected to a single step normalization-subtraction step, with PCR-derived single strand anti-sense DNA drivers produced from these libraries[45]. Normalization refers to low CoT hybridization (CoT=2) with drivers produced from the “self” library and aims to decrease the representation of highly expressed mRNAs. Subtraction refers to high CoT hybridization (CoT=100-200) with drivers produced from different libraries. Hybridized double stranded cDNAs were removed with two passes through a Hydroxyapatite (HAP) column. Non-hybridized, single stranded cDNAs were converted to double stranded and were subsequently electroporated into DH10b bacterial cells, where only tester cDNAs are able to propagate (due to the presence of a replication origin and antibiotic resistance)[45].

High-throughput sequencing and sequence analysis. Colony picking, cDNA sequencing and sequence analysis were performed essentially as previously described [46-48]. Sequences were filtered for primer and vector sequences [48] and masked for rodent specific and mammalian wide repeats with RepeatMasker (http://www.phrap.org). EST clustering was performed with stackPACK and d2_cluster (word size 6, similarity cutoff 0.98, minimum sequence size 50, window size 200)[49,50]. Homology searches with known genes were performed with BLAST [51,52] at Unigene (http://www.ncbi.nlm.nih.gov/UniGene), FANTOM2 (http://fantom2.gsc.riken.go.jp) [53] and SWISSPROT (http://www.ebi.ac.uk/swissprot/) databases. BLAST results were associated with Gene Ontology (GO) terms (http://www.geneontology.org) at http://source.stanford.edu/cgi-bin/sourceSearch (for Unigene), ftp://ftp.ebi.ac.uk/pub/databases/GO/goa/SPTR/gene_association.goa_sptr.gz (for SWISSPROT) and http://fantom2.gsc.riken.go.jp (for FANTOM2). The detailed results (including clone numbers, clusters/genes, blast results and E values, accession numbers and GO assignments) can be found in the corresponding author's web site, at http://www.fleming.gr/en/investigators/Aidinis/data.html. Most of the sequencing data, have been already submitted to public databases [47], in the context of the ongoing FANTOM (Functional annotation of the mouse) project [53,54].

Differential gene expression statistical analysis of subtractive libraries. The differential gene expression/abundance among the different subtracted cDNA libraries was calculated with a single statistical test, designed especially for this purpose [55]. Essentially, the used formula is the entropy of a partitioning of genes among cDNA libraries and is described by an R-value, which is the log likelihood ratio statistic, that follows (2Rⱼ) an asymptotic χ² distribution[55]. The formula for the statistic Rⱼ for the j^{th} gene is given by the expression:

$${R}_{j} = {\sum }_{i = 1}^{m} {χ}_{i , j} log ( \frac{{χ}_{i , j}}{{N}_{i} {f}_{j}} )$$

$$where$$

$${f}_{j} = \frac{{\sum }_{i = 1}^{m} {χ}_{i , j}}{{\sum }_{i = 1}^{m} {N}_{i}}$$

where m is the number of cDNA libraries, Nᵢ is the total number of clones sequenced in the i^{th} library, χ_{i,j} the counts (transcript copies) of the j^{th} gene in the i^{th} library, and fᵢ is the frequency of gene transcripts copies of the j^{th} gene in all the libraries.

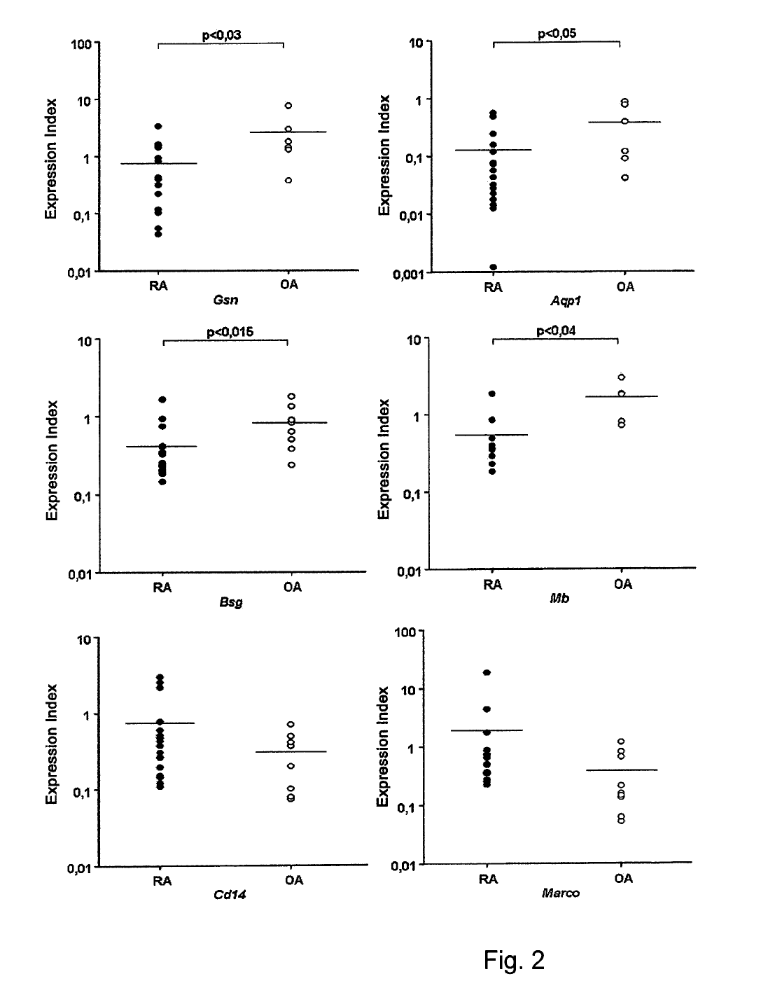

The significance of the R statistic was established utilizing a re-sampling method, which tries to establish an “optimal” cut-off value using simulated library datasets based on the observed counts. The method proceeds as follows: 1) For each and every gene, the common gene transcript frequency, fⱼ is calculated with the help of the formula above. This number corresponds to the expected frequency of gene transcripts under the null hypothesis of no difference in the abundance of gene j across all libraries. 2) The parameters of the Poisson distributions giving the sampling distribution of clone abundance for each gene and library are calculated under the null hypothesis. This parameter is equal to Nᵢ fⱼ for gene j in library i, where Nᵢ is the total number of clones (taking into account ALL genes) sequenced in library i and fⱼ was calculated in Step 1. This value is equal to the expected absolute number of clones of the j^{th} gene in the i^{th} library under the null hypothesis. 3) Then, for each library compared and each gene, a random number is generated from the Poisson distribution of step 2. This number is a simulated count compatible with the null hypothesis i.e. gene frequency for a particular gene is the same across all libraries. This step uses the random generating functions of the computer to create an artificial dataset corresponding to performing the actual experiment of library creation and analysis for each of the libraries compared under the null hypothesis. 4) For gene j in the artificial dataset of Step 3 we calculated the test statistic R by substituting for χ_{i,j} the random number generated in Step 3, for fⱼ the frequency calculated in Step 1 and for Nᵢ the total number of clones sequenced. 5) The R values calculated in Step 4 are sorted in descending order, and for a range of values for specificity (i.e. the true negative rate) we find the corresponding R statistic value. These are by definition true negatives since they were obtained from libraries under the null hypothesis. 6) Steps 3-5 are repeated 1000 times allowing us to construct the histogram shown in Supplementary FIG. 2a. These histograms depict the distribution of the R value as a function of the required true negative rate cutoff. The mean of this (empirical) distribution is used as the R-value cutoff for the analysis of the experimental dataset. 7) The experimental dataset R values are computed for all the comparative libraries using the observed clone counts. Genes with R values greater than the previously established cutoff are considered to be differentially regulated between the libraries. Calculations were performed in the Computer Algebra System Mathematica 4.2 (www.wri.com) with the MathStatica add-on [56], notebooks available under request.

High density oligonucleotide array hybridization. cRNA probes were generated from 5 μg of total RNA and hybridized to the Mu11K (subunits A and B) chip set according to the manufacturer's instructions (Affymetrix, Santa Clara, Calif., USA) and as previously described [7]. The chip set is designed to collectively recognize 13179 distinct murine transcripts, where the expression level of any given gene is interrogated by 40 oligonucleotides, 20 with a perfect match sequence (PM) and 20 carrying a single mismatch (MM). Hybridized chips were washed and stained in a Fluidics Station (Affymetrix) following standard protocols; subsequently fluorescence intensities were read by an Affymetrix scanner. All MIAME-compliant microarray data can be downloaded from the ArrayExpress database (accession number: E-MEXP-255), as well as from the corresponding author's web site (http://www.fleming.gr/en/investigators/Aidinis/data.html).

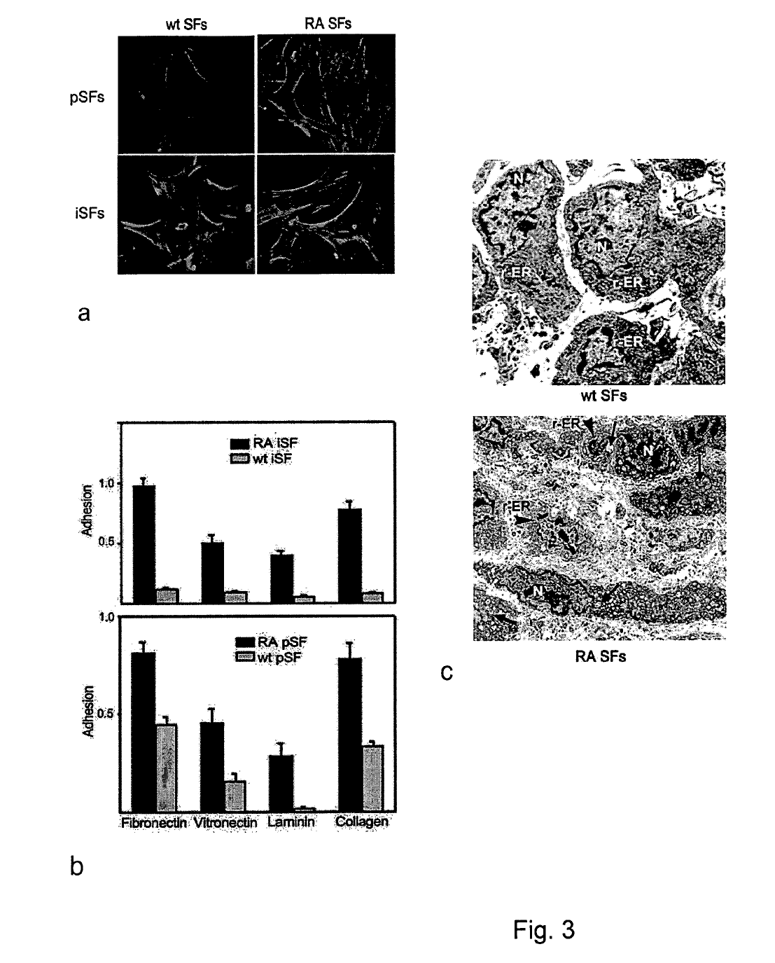

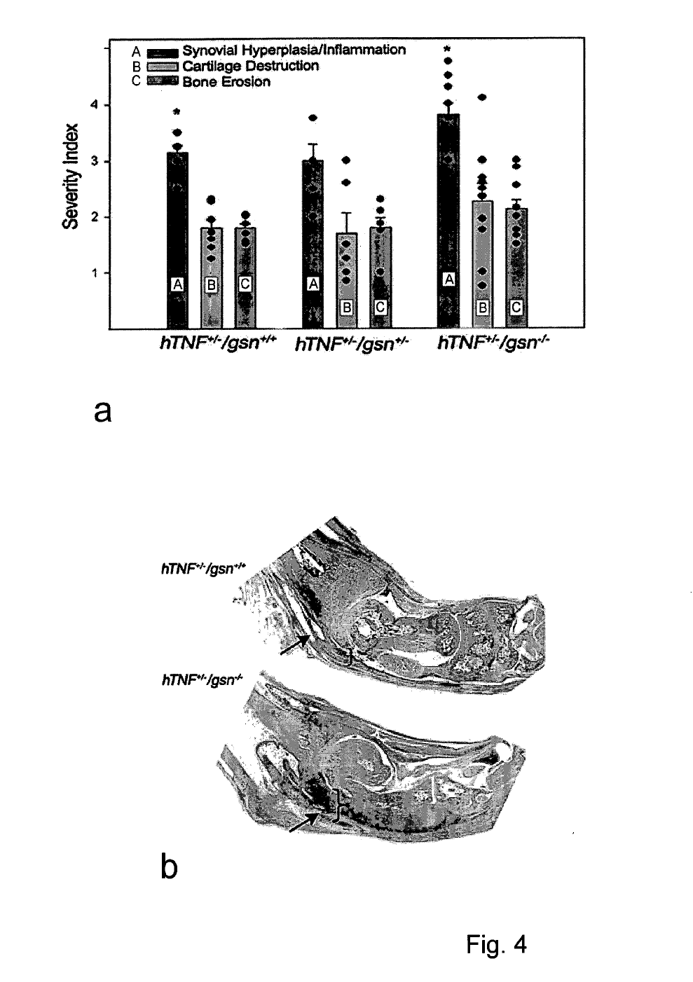

Microarray data pre-processing and normalization (outlined in Supplementary FIG. 3). Low-level analysis of the resulting scanned image (as background subtraction and computation of individual probe cell intensities) was automatically performed by MicroArray Suite 5.0 (MAS5) software (Affymetrix). Briefly, the quantitative level of expression (Signal value), as well a qualitative measure of expression (Detection call), of any given gene is calculated by proprietary Affymetrix algorithms from the combined, background-adjusted hybridization intensities of 20 PM and 20 mM oligonucleotides (probe set). All signal values from all chips in the experiment were scaled to reach target intensity (TGT) of 500 or 2500, following Affymetrix recommendations for the individual chip sets used. Scaled values were then submitted to a normalization step that has the aim to force each chip subunit's Signal distribution to have an identical overall shape across chips of the same subunit. Gene expression values were reorganized into two distinct data matrices, one for each chipset subunit (A,B), where rows and columns represented genes and chips respectively. The columns of the data matrix were normalized to have the same quantiles using BioConductor [57]. Each quantile of each column was set to the mean of that quantile across arrays (Supplementary FIG. 4a). The rows of the two data matrices were then merged and the columns of the resulting data matrix were split according to the corresponding animal model of rheumatoid arthritis (Tg197, TNF^{ΔARE}) and to the type of sample (SF or WJ), thus obtaining a total of 4 distinct data matrices. Finally, genes that were not called Present (detection call) or that had a normalized Signal lower than one fifth of the TGT in at least one sample were not further considered. Subsequent analysis was therefore performed on the remaining 8021 probe sets.

Microarray data analysis. Comparisons of Affymetrix chips hybridizations and real time PCR have indicated that chip analyses are accurate, reliable and underestimate differences in gene expression [58]. Nevertheless, in order to assess the system's performance, the reproducibility of the (technical) duplicate samples was examined, in terms of Pearson correlation coefficient of normalized Signals, as well as Detection concordance (expressed as the percentage of probe sets that were consistently detected with a “Present” or an “Absent” call in both chips). As it can be seen in Supplementary FIG. 4b, Affymetrix data were highly reproducible and reliable.

As previously reported [59,60], the significance of fold changes (that are commonly used as a measure of differential expression) is highly dependent on the expression level of the corresponding genes. Therefore, in order to identify genes that are significantly differentially expressed, we decided not to use a fixed fold change threshold, but instead model intensity-dependent fold changes between replicated chips using a variant of a previously described approach [61]. Briefly, we first calculated fold changes in pair-wise comparisons of replicated chips representing the transcriptome of the same sample type (SF or WJ) measured under the same experimental condition using the following definition:

FC=|x₁−x₂|/min(x₁, x₂)

where xᵢ was the normalized expression level of a given gene in the i^{th} chip. Genes were then ranked according to the minor of their two expression values (i.e. min(x₁, x₂)) and the overall expression range was partitioned into 10 intervals, each containing an equal number of probe sets. In each partition the median of all (min(x₁, x₂)) values as well as the 90^{th} and the 95^{th} percentile fold change were determined, thus obtaining 10 distinct modeling points for each sample type (SF or WJ) and for each of the two significance levels (90% or 95%). Based on the observation that measurement variability of high-density oligonucleotide microarrays depends on signal intensity following a power law [11], a continuous Fold Change Model (FCM) was derived from the 10 modeling points using a least squares linear fit in log-log plots. The following modeling parameters have been obtained for the two FCM (Supplementary FIG. 4c): a slope of −0.327 and an intercept of 2.356 for the SF FCM and a slope of −0.346 and an intercept of 2.678 for the WJ FCM. This means that WJ are intrinsically more variable than SF (as expected). Using slopes (a90% or a95%) and intercepts (b90% or b95%) of the resulting regression curves, we could obtain for each given sample type (SF or WJ) and for any given minimum expression level a 90% as well as a 95% significance threshold:

FC_{90%}=min(x₁, x₂)^{a90%}*e^{b90%}

FC_{95%}=min(x₁, x₂)^{a95%}*e^{b95%}

For each of the 4 data matrices (i.e. Tg197/SF, TNF^{ΔARE}/SF, Tg197/WJ, TNF^{ΔARE}/WJ) an observed average fold change between experimental conditions (diseased or normal) was calculated for each gene in the following way:

FC_{obs}=|μ₁−μ₂|/min(μ₁, μ₂)

where μᵢ was the average expression level of a given gene in the i^{th} experimental condition. If no replicates were available, the single expression value of the given gene was used instead of the average. For each gene, this observed fold change was then compared to the fold change that was expected at 90% (or 95%) significance level using the corresponding “sample type”-specific FCM, given the observed values of μᵢ of that gene:

FC_{90%}=min(μ₁, μ₂)^{a90%}*e^{b90%}

FC95%=min(μ₁, μ₂)^{a95%}*e^{b95%}

Finally, genes with FV_{obs}>FC_{90%} (or >FC_{95%}) were selected as differentially expressed at a significance level of 90% (or 95%, respectively).

Visualization of quantitative trait loci (QTL) and gene-expression data was performed with Expression view [62] at http://ensembl.pzr.uni-rostock.de/Mus_musculus/expressionview. QTL data were derived from Serrano-Fernadez et al [63].

Functional clustering and determination of statistical significance. Biological annotation, in the form of Gene Ontology (GO) “Biological process” term [17], for each of the genes (probe sets) in table 2, was obtained from the NetAffx portal (http://www.affymetrix.com). We then calculated the observed GO term frequencies, as means to discover deregulated functions (Supplementary Table 3). The statistical significance for GO term frequencies was determined essentially as has been previously described [64]. Briefly, the hypergeometric distribution was used to obtain the chance probability of observing a given number of genes annotated in NetAffx with a particular GO term and thus calculating appropriate p-values. More specifically, the probability of observing at least (k) probe sets annotated with a particular GO term within a list of selected probe sets of size (n) was calculated [64] as:

$$P = 1 - {\sum }_{i = 0}^{k - 1} \frac{( \begin{matrix}f \\ j\end{matrix} ) ( \begin{matrix}g - f \\ n - 1\end{matrix} )}{( \begin{matrix}g \\ n\end{matrix} )} ,$$

where (f) was the total number of genes within a functional category and (g) was the total number of probe sets on the chip set (13,179).

Adhesion assays were performed on Cytometrix adhesion strips (Chemicon) coated with human fibronectin, vitronectin, laminin and collagen I, according to the manufacturer instructions.

Immunofluorescence. Cells were fixed using 4% paraformaldehyde/PBS and stained by standard methods. For visualizing F-actin cells were stained with Alexa594-phalloidin (Molecular Probes). P-tyrosine was detected using the 4G10 antibody (Upstate Biotechnology), FAK was detected using a monoclonal antibody (clone 77, Transduction Laboratory). Antibodies against gelsolin were raised by immunizing rabbits with recombinant mouse gelsolin, immune serum was used at 1:500 dilution. For western blot analysis, 10 μg of total cell protein was separated by SDS/PAGE, transferred to Immobilion-P and probed for gelsolin and re-probed for actin as an internal loading control.

RT-PCR. Total RNA were extracted from synovial fibroblast and whole joints tissue using TRIzol reagent (Life Technologies, Rockville, Md.) in accordance with the manufacturer's instructions. RNA yield and purity were determined spectro-photometrically at 260/280 nm. 1^{st} strand cDNA synthesis was performed using the MMLV reverse transcriptase and oligo-dT₁₅ (Promega).

Semi-quantitative PCR was performed by 20-25 cycles of denaturation at 95° C. for 30 s, annealing at 57-62° C. (depending on the Tm of each individual set of primers) for 30 s and extension at 72° C. for 1 min, in a final volume of 20 μl. The products were separated by electrophoresis on 1.5% agarose gel and stained with ethidium bromide. Product intensity was quantified with GelWorcs 1D Advanced (v.4.01) and normalized to the intensity of B2m and/or L32. The primers were selected to span two exons, while the two control primers were chosen from the Primer Bank database (http://pga.mgh.harvard.edu/primerbank/). Primer sequences (5′->3′, s: sense; as: anti-sense) and product sizes (in bp) were as follows:

| | |
|---|---|
| AqpI | (s: TCACCGGCAACTTCTCAAAC as: AGCTCTGAGACCAGGAAACA, 400), |
| Bsg | (s: ATGAGAAGAGGCGGAAGCCA as: CCACTCCACAGGGCTGTAGT, 426), |
| CD14 | (s: CAATCCTGAATTGGGCGAGA as: CGAGTGGGATTCAGAGTCCA, 100), |
| CDC42 | (s: AAGTGGCCCAGATCCTGGAA as: AGCACTGCACTTTTGGGGTT, 380), |
| Gsn | (s: TGCAGGAAGACCTGGCTACT as: ATGGGTTGGTCCTTACTCAG, 300), |
| Hp | (s: GAAGCAATGGGTGAACACAG as: GGGGTGGAGAACGACCTTCT, 331), |
| Marco | (s: CACAGGAATTCAAGGACAGA as: ATTGTCCAGCCAGATGTTCC, 397), |
| Mglap | (s: CAGTCCCTTCATCAACAGGA as: CTGCAGGAGATATAAAACGA, 274) |
| Mb | (s: TCACACGCCACCAAGCACAA as: TGGGCTCCAGGGTAACACTG, 354), |
| Ptgis | (s: TCACAGATGACCACACTCCC as: GCAGTAGGACGACAAATTGT, 403), |
| B2M | (s: TTCTGGTGCTTGTCTCACTGA as: CAGTATGTTCGGCTTCCCATTC, 104) |
| and | |
| L32 | (s: TTAAGCGAAACTGGCGGAAAC as: TTGTTGCTCCCATAACCGATG, 100). |

Quantitative real-time PCR was performed using the iCycler iQ Real-Time detection system and the IQ™ SYBR Green Supermix (Bio-Rad Laboratories), according to the manufacturer's instructions, for 4 min at 94° C. and 40 cycles of denaturation\ annealing (50 s at 95° C.; 50 s at 57-62° C.). Primers were chosen from exons separated by large introns, and the PCR quality and specificity was verified by melting curve analysis and gel electrophoresis. Values were normalized to B2m and/or L32 (using the same primers as for the semi-quantitative PCR). Mouse (m) and human (h) primer sequences and expected lengths were as follows:

| | |
|---|---|
| mAqp 1 | (s: TCACCCGCAACTTCTCAAAC as: TCATGCGGTCTGTGAAGTCG, 123), |
| mGsn | (s: TGCAGGAAGACCTGGCTACT as: TCGATGTACCGCTTAGCAGA, 130). |
| hAqp 1 | (s: CTCCCTGACTGGGAACTCG as: GGGCTACAGAGAGGCCGAT, 182), |
| hBsg | (s: TTCCTGGGCATCGTGGCTGA as: GCGGACGTTCTTGCCTTTGT, 159), |
| hCd14 | (s: CGGCGGTCTCAACCTAGAG as: GCCTACCAGTAGCTGAGCAG, 142), |
| hcdc42 | (s: AATTGATCTCAGAGATGACC as: TTTAGGCGTTTCTGTGTAAG, 150) |
| hGsn | (s: GGTGTGGCATCAGGATTCAAG as: TTTCATACCGATTGCTGTTGGA, 199), |
| hMarco | (s: TGGGACGAGATGGAGCAAC as: CCCTTAGTTCCAGTTTCCCCTT, 193), |
| hMb | (s: TTGGTGCTGAACGTCTGGG as: CTGTGCCAGGGGCTTAATCTC, 249), |
| hB2M | (s: CTGAAAAAGATGAGTATGCC as: ACCCTACATTTTGTGCATAA, 202) |
| and | |
| hL32 | (s: TTAAGCGTAACTGGCGGAAAC as: GAGCGATCTCGGCACAGTAA, 210) |

Cycle threshold (Ct; the first cycle that amplification can be detected) values were obtained from the iCycler iQ software for each gene of interest (GOI) and control housekeeping genes (HKG; L32 and/or b2m), together with amplification efficiencies (η; 80-120%). For the mouse samples, we calculated the relative expression of the samples (S) to wt controls as reference samples (R) using the gene expression—relative quantification Microsoft® Excel add-on macro (Bio-Rad Laboratories) that utilizes the following formulas: Relative expression=2·^{(SΔCt·RΔCt)}, where ΔCt=GOI Ct-HKG Ct. For the human samples, Ct values were converted to concentration values (ng/ml) utilizing the standard curve made by serial dilutions (in duplicates) of a reference sample. Values were normalized to the corresponding values of the reference (housekeeping) gene(s) and presented (in logarithmic scale for visualization purposes) as expression index.

Arthritic score and Histopathology Paraffin-embedded joint tissue samples were sectioned and stained with haematoxylin and eosin. Arthritic histopathology was assessed (in a blinded fashion) separately for synovial hyperplasia, existence of inflammatory sites, cartilage destruction and bone erosion using a semi-quantitative (0-5) scoring as described previously [65].

Transmission electron microscopy. Ankle joints (dissected from the right hind leg of each mouse—3 Tg197 and 3 wt) were split open longitudinally through the midline between the tibia and the talus, were prefixed with 2.5% glutaraldehyde in PBS (pH 7.4) O/N. After postfixing with 1% osmium tetroxide in PBS for 2 h, they were dehydrated in a graded series of ethanol and processed into Araldite. Semithin sections (1.0μ) were cut and stained with methylene blue to observe the orientation under the light microscope. Ultrathin sections (80-90 nm) were then cut with an ultramicrotome (Riechert-Jung Ultracut E), mounted on copper grids, counterstained with uranyl acetate and lead citrate, and evaluated through electron microscope (CM120 Biotwin, FEI Company).

### Results

To discover genes and cellular pathways that participate in the pathogenesis of RA on a large scale, we performed a twin high throughput approach, comprising of two entirely different methodologies, governed by different constrains and analyzed by different statistics. Total RNA samples were extracted from whole joints (WJ) and synovial fibroblast (SF) ex vivo cultures, isolated from 6 weeks old arthritic (RA; disease severity index 3, see FIG. 4) mice (Tg197, hTNF+/−)[5] and their normal (wt) littermates. Each sample (out of 4: RA SF, wt SF, RA WJ, wt WJ) consisted of equimolar amounts of RNA pooled from 4 (2 male and 2 female) mice (16 mice altogether), thus equalizing (to an extent) biological diversity. Similar samples (biological replicates: different extractions, same pooling strategy) were used for both the creation of subtractive cDNA libraries, as well as for the hybridization of oligonucleotide microarrays.

Subtractive cDNA libraries and large scale sequencing. Full-length, cDNA libraries were constructed with the cap-trapper method, by cloning in the λ-FLCI vector, as described in Materials and Methods. Following solid phase amplification and plasmid excision, the four different cDNA libraries (RA SF, wt SF, RA WJ, wt WJ) were normalized and subtracted to each other, as outlined in Supplementary FIG. 1a. Normalization refers to low CoT hybridization of the tester library with drivers produced from the “self” library and aims to decrease the representation of highly expressed mRNAs. Subtraction refers to high CoT hybridization with drivers produced from different libraries and aims to remove mRNAs common in both populations. Due to experimental design, the resulting subtracted libraries (L0, L1, L2 and L7) contain cDNAs from the tester cDNA library only and therefore constitute libraries of upregulated genes in the corresponding tester library (or of down-regulated genes in the driver library). 27511 cDNAs/clones from the subtracted libraries were sequenced and clustered to 9176 clusters/genes, as described in Materials and Methods and summarized in Supplementary FIG. 1b. Each gene was then annotated through BLAST homology searches at Unigene, FANTOM and SWISSPROT databases, as described in Materials and Methods. In summary, among the 9176 genes found, 7977 corresponded to known genes, while 1199 had sequences not reported previously. Each gene was represented by a different number of clones in almost all libraries, directly proportional to subtraction efficiency and transcript abundance. The relative distribution of each gene in each library is the true measure of differential expression, which can be obscured by sampling errors arising by chance when the clones are selected. Therefore and in order to identify the truly differentially expressed genes, a likehood value (R) was assigned to each gene from pair-wise comparisons of the relative libraries (SF/L0L1, WJ/L2L7). The statistical significant thresholds were then calculated, as described in Materials and Methods and presented in Supplementary FIG. 2. Two significance levels were selected (summarized in Table 1a): a very high one (99.99%; p≦0.0001) to report the results independently, and a lower one (99%; pa 0.01) for the comparison with the corresponding results from the DNA microarrays hybridizations. Known (with a Unigene cluster ID) differentially expressed genes at 99.99% significance level are presented in Supplementary Table 1.

Oligonucleotide microarray hybridizations. Fluorescent-labeled cRNA probes made from similar (biological replicates, see above) samples of total RNA (RA SF, wt SF, RA WJ, wt WJ) were utilized to hybridize (in duplicates) the Affymetrix Mu11K oligonucleotide DNA chipset (8 chipsets, 16 chips total). Furthermore, similar samples (equimolar amounts of RNA pooled from 4-2 male and 2 female—6 weeks old arthritic mice; severity index 3) of total RNA (from SF and WJ) from another animal model of Arthritis (spontaneous, knock-in, TNFΔARE+/−)[8] were used for additional chip hybridizations (4 chipsets, 8 chips total). The MIAME-compliant [9] microarray data (which can be found at the ArrayExpress database [10], accession number: E-MEXP-255) were normalized and analyzed as outlined in Supplementary FIG. 3 and as described in detail in Materials and Methods and supplementary FIG. 4. Differentially expressed genes (DEG) were selected, as described in Materials and Methods, utilizing a sample-specific Fold Change Model (FCM)[11] at different significance levels (90%/p≦0.1 and 95%/p≦0.05), where selected DEG have always an observed fold change higher than the expected fold change. The results for both animal models are summarized in Table 1b. Sample specific differentially expressed genes common in both animal models (significance: 95%, p<0.05) are presented in Supplementary Table 2.

Cross platform comparison and validation. Both differential expression analysis methods presented above produced lists of deregulated genes of high statistical significance. In order to validate the results independently and in order to avoid performing numerous RT-PCRs, differential expression results by both platforms of analysis were compared to each other, for the same animal model (Tg197, hTNF+/−) and sample type (WJ, SF), as well as for the same direction of deregulated expression (up or down). The comparison, at significance levels 99% for the libraries and 90% for the microarrays (selected based on similar output gene numbers), was performed through the NetAffx database (www.affymetrix.com). Remarkably (since there have been very few examples of expression profiling cross-platform overlaps[12,13]), 46 (15 for SF; 31 for WJ) genes were commonly predicted as up/down-regulated by both methods (Table 2) (with a combined p value of 0.001).

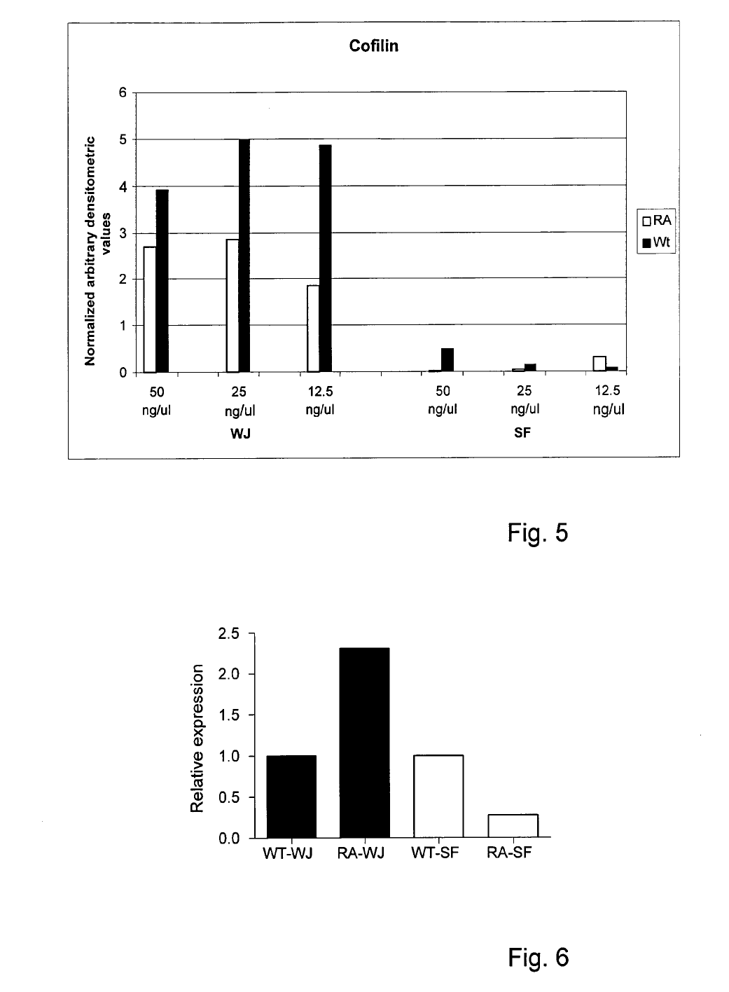

In order to verify the validity of the twin high throughput approach and to prove that the reported gene list is self-validated, a number of the predicted deregulated genes, were confirmed with different methods. 40 genes were also predicted deregulated in the knock-in disease model (TNFΔARE+/−, Table 2). 20 genes were previously reported in the literature to be associated with RA (Supplementary FIG. 5; summarized in Table 2), as these were identified through the Biolab Experiment Assistant™ text-mining software (BEA, BIOVISTA, Greece). 11 representative genes (SF: Gsn, Aqp1, mglap, cdc42 hom., Hp; WJ: Marco, Hp, CD14, Mb, Bsg, Ptgis) were further confirmed by semi-quantitative (SQ) RT-PCR (Supplementary FIG. 6 a,b,c, summarized in Table 2). All SQ-RT-PCRs were performed in the linear-range of the reaction (at three different concentrations normalized against housekeeping genes) in biological replicates (same pooling strategy) of the samples used for both the subtractive libraries and the microarrays hybridizations. Representative genes were selected on the basis of i) different sample source: 6 from WJs and 5 from SFs, ii) different direction and degree of deregulated expression: 6 upregulated and 5 down-regulated and iii) biological interest and potential follow up. Moreover, two of them (SF: Gsn, Aqp 1) were also confirmed by Real-time RT-PCR (Supplementary FIG. 6e, summarized in Table 2).

In an attempt to combine gene expression analysis with genetic linkage analysis, all differentially expressed genes were mapped to the chromosomes, together with the known Quantitative Trait Loci (QTLs, chromosomal regions/genes segregating with a quantitative trait) for an induced animal model of Arthritis, Collagen Induced Arthritis (CIA)[14]. As graphically represented in FIG. 1 (and summarized in Table 2), eight genes mapped to CIA QTLs (WJ: Ctss, Pitpnm, Ncf1, Psmb8, Siat8e, SF: Rab14, Aqp1, and Gsn), proving the validity of the selected gene list even further. The expression level of seven of the genes found deregulated in the arthritic mice and confirmed by RT-PCR in mouse samples, was also examined in human patient's RNA samples with Real-time RT-PCR analysis. Due to the lack of normal (wt) human synovium samples, we compared the expression of 19 RA samples with 8 Osteoarthritic (OA) samples as controls, a consensus strategy for differential expression analysis in Arthritis [15,16]. As shown in FIG. 2 and summarized in Table 2, the deregulated expression of six (out of seven assayed) of the genes was confirmed in humans as well, including four with high statistical significance (Gsn, Aqp1, Bsg, Mb,), thus extending the validity and utility of the mouse model generated deregulated gene list to the human disease.

Arthritic synovial fibroblasts have a rearranged cytoskeleton. The twin high throughput expression profiling approach described above, yielded a large number of disease implicated, deregulated genes, of high statistical significance. Furthermore and in order to: i) prove the validity and extend the utility of the expression data analysis even further, ii) to infer deregulated biological functions from the gene expression data and iii) to define functional criteria for further gene selection, the selected genes were annotated in the form of the Gene Ontology (GO)[17] term “Biological process”. GO term frequencies in the selected gene lists were then calculated and their statistical significance was estimated, as described in Materials and Methods. As shown in Supplementary Table 3, predicted deregulated functions in SFs include, as expected, collagen catabolism, complement activation, immune and stress response. Interestingly, 5 out of 26 statistical significant (p<0.01) deregulated GO functions concerned (directly or indirectly) the actin cytoskeleton, suggesting that arthritic SFs have a rearranged actin cytoskeleton. In order to confirm the prediction, F-actin was visualized in vitro on arthritic as well as wt SFs (both primary and immortalized). As evident from FIG. 3a, arthritic SFs exhibit pronounced stress fibers, thus validating the in silico, expression-based hypothesis.

Stress fibers within fibroblasts allow them to exert tension on the Extra Cellular Matrix (ECM) surrounding them—an essential process in wound healing. It is well expected that differences in the actin cytoskeleton reflect altered ECM attachment properties and/or vice versa. Indeed, arthritic SFs were shown to adhere to different proteins of the ECM (fibronectin, vitronectin, laminin and collagen) with increased affinity in vitro (FIG. 3b).

Attachment to the ECM (and associated cytoskeletal changes), mediated mainly through engagement and clustering of transmembrane integrin molecules, largely define cell shape and morphology, as well as their behavior and fate. Increased adhesion to the ECM is expected to lead to a more elongated shape. In order to confirm the increased adhesion of the arthritic fibroblasts in vivo, we examined ankle joints from arthritic or wt littermate mice on an ultrastructural level with transmission electron microscopy (FIG. 3c). In wt mice, SFs contained prominent nuclei, abundant rough endoplasmic reticulum (r-ER) and mitochondria of different shapes and sizes. In contrast, remarkable modification of the SFs was noticed in the joints of arthritic mice, where randomly flattened cells had (most of them) elongated shape, characterized by dilation of the r-ER and by swollen mitochondria with distorted cristae.

We thus found a new pathogenic mechanism in RA: The promotion of actin polymerization and rearrangement of the actin cytoskeleton. Gelsolin (Gsn) is a gene that maps in one of the CIA QTLs (FIG. 1) and whose expression was found downregulated in arthritic SFs by both subtractive libraries and microarray hybridizations. Its deregulated expression was confirmed with Real-Time RT-PCR in both mouse (Supplementary FIGS. 5b, c and e) and human samples (FIG. 2), as well as with western blot in immortalized SFs (Supplementary FIG. 5d). The gene encodes for an actin binding protein with filament severing properties [18] and Gsn−/− fibroblasts have excessive actin stress fibers [18], very similar to the ones observed in arthritic SFs (FIG. 3a). In order to prove the involvement of cytoskeleton organization in the pathogenesis of RA and to highlight the role of gelsolin in it, the arthritic mice (Tg197, hTNF+/−) were mated with the gsn knock out mice (gsn−/−)[18]. Predictably, knocking out gsn expression from SFs would promote RA pathogenesis by inhibiting the severing activity of gsn. As shown in FIG. 4, abolishing gsn expression resulted in hyperplasia of the synovial membrane and exacerbation of the disease.

### Example 2

### Materials and Methods

Unless mentioned herein under example 2, the materials and methods of example 1 where used.

### SQ and Q-RT-PCR Analysis of Mouse Cofilin 1 and VASP

Details on cell culture, RNA extraction from both whole joint tissue and ex-vivo synovial fibroblast cell cultures and RT-PCR conditions can be found at Aidinis et al, 2005. The primers specific for mCofilin 1 were s:TGGGTGAAACTCTGGGAGAT as: CCATAGTGGAAGCCTGATGC, 399 bp and for mVASP s: CTTGCCAAGGATGAAGTCG as: GCTTCACCCTCTCCAAGT, 98 bp.

### Wound-Healing Assays

Cells cultured at confluency (3×10⁵ per well) were seeded in 6-well plates (in DMEM supplemented with 10% FCS and 100 Units/ml of penicillin/streptomycin at 37° C., 5% CO₂) and allowed to adhere for 24 h. The following day cells were treated with 10 μg/ml Mitomycin C (Sigma) in culture medium at 37° C. for 2 h, to block cell proliferation. Assays of wound healing were performed by scrapping the cell monolayer with the edge of a pipette tip, forming a straight line. Cells were then allowed to continue to grow and pictures were taken at 0, 24 and 48 h, after the scrapping.

### Apoptosis Assays

Confluent mouse synovial fibroblasts were washed twice with phosphate-buffered saline (PBS) and placed in DMEM without serum for 24 h. The following day 1.5×10⁴ cells were plated into each well of a 96-well flat-bottom plate (Costar, Corning Inc.) in 100 μl of media plus serum. After 24 h, cells were treated with 12.5-100 ng/ml TNFα plus 0.5-3 μg/ml cyclohexamide (CHX), 30-300 μM Etoposide and 0.01-1 μM Staurosporine, for 24 h at 37° C. overnight. Cell survival was determined using the crystal violet assay. The absorbance at 570 nm was determined on a microplate reader (SPECTRAmax PLUS³⁸⁴, Molecular Devices, Sunnyvale, Calif., USA), as previously described (Aidinis et al, 2003). Results are expressed as the means±S.E. of one representative experiment performed in triplicate. Statistical analysis was performed using Student's t test, with a level of significance set at p<0.05.

### Drug Treatment—In Vitro Assays

Disruption of actin cytoskeleton was induced with 4 μg/ml of Latrunculin A (Sigma) for 2 h at 37° C. as previously described (Spector et al, 1989). As control of TNFα involvement in actin-cytoskeleton organisation 1 μg/ml of anti-TNF (Infliximab) was added on the cells for the same period of time.

### Results

### Validation of Deregulated Genes (DEGS), Identified by Microarrays, in the Tg197 Mouse Model Using SQ-RT-PCR

Total RNA was extracted from 5-8 Tg197 mice (from both whole joint tissue and ex-vivo synovial fibroblast cell cultures) and pooled. For the comparison group, total RNA was extracted from wild-type litter mates (from both whole joint tissue and ex-vivo synovial fibroblast cell cultures) and pooled. The results are depicted in table 3.

Preferred proteins that affect cytoskeleton arrangement include actin binding proteins, controlling the nucleation, assembly, disassembly, crosslinking of actin filaments and/or the size, localization and dynamics of the un-polymerized actin. A protein of table 4 is preferred in the present invention. A particularly preferred protein includes a protein expressed by the gelsolin gene, VASP and/or Cofilin (Galler et al, 2006; Hotulainer et al, 2005; Paavilainen et al, 2004). Others, like Autotaxin and SHIP2 (Song et al, 2005; von Wichert et al, 2003). These proteins are major players in signaling pathways leading to re-organization of the actin cytoskeleton.

Cofilin 1 (non-muscle cofilin) promotes actin dynamics by depolymerising and severing actin filaments, in a similar manner like gelsolin (Hotulainer et al, 2005). As FIG. 5 shows, cofilin 1 was down-regulated in both Tg197 whole-joint and synovial fibroblasts compare to wild-type ones.

In order to evaluate the gene expression status of VASP in Tg SF's we have performed Q-RT-PCR in both whole joint samples and ex-vivo synovial fibroblast cells culture (same material as for the SQ-RT-PCR). Surprisingly, VASP is found to be over-expressed in the whole joint of Tg mice compare to wild-type littermates, while it is under-expressed in the SF's (FIG. 6). This finding although unexpected could probably suggest the presence of another cell type within the arthritic joint that might be linked with VASP expression. Interestingly, there are some indications of the possible presence of myofibroblasts in the arthritic synovium (Aidinis et al, 2005). Myofibroblasts are differentiated fibroblasts with a crucial role in manifesting tension during wound-healing and pathological contracture (Tomasek et al, 2002).

KO mice for genes known to be involved in the cytoskeleton or crossed with Tg197 and/or with Collagen VI-Cre knockout with the purpose of (i) exacerbate, (ii) ameliorate the disease and (iii) make the disease specific for synovial fibroblasts.

VASP is a key player in the actin cytoskeleton as it is shown in vitro to promote F-actin formation while in vivo regulates and promotes actin polymerization (Hauser et al, 1999). In the present invention a cross of VASP KO with Tg197 is expected to lead to amelioration of the disease.

Cofilin depolymerises and severs actin filaments, in a similar manner like gelsolin, in the present invention a crossing of Cofilin KO with Tg197 is expected to result in exacerbation of the disease (Gurniak et al, 2005).

LST-1 was found to be over-expressed in whole joints from Tg197 (Aidinis et al, 2005), while in vitro studies have correlated this gene with a function in cytoskeleton, by creation of long filopodia and thus accumulation of F-actin (Raghunathan et al, 2001). Creation of a KO of this gene and crossing with Tg197 provides further evidence of the involvement of this gene in cytoskeleton-related rheumatoid arthritis. RhoA, Rac and Cdc42 belong to the Rho family of GTP-binding proteins known to induce actin polymerization (Lamarche et al, 1996). Conditional ablation of these genes (Chrostek et al, 2006) and crossing with ColVI-Cre and Tg-197 would ameliorate the disease specific for synovial fibroblasts. A similar outcome is predicted from crosses with the serum response factor (SRF) KO, which is a transcription factor regulating the expression of several actin-cytoskeleton genes (RhoA, Vinculin, Actin) and it's ablation was shown to result in low F-actin levels and a reduced adhesive and migratory capacity (Wiebel et al, 2002).

A conditional KO model for Autotaxin (ATX) will be also utilized to further support the role of cytoskeleton re-modeling in RA. Activated hyperplastic RA Synovial Fibroblasts (SFs) exhibit reorganization of their actin cytoskeleton and deregulated ECM adhesion that could explain the tumor-like behavior of the arthritic synovium. Lysophosphatidic acid (LPA), the enzymatic product of autotaxin (ATX)—a lysophospholipase D highly expressed in a variety of tumors, is an important lipid mediator that has been found to induce cytoskeletal reorganization, proliferation and migration in many cell types. Preliminary studies have shown that the expression of ATX is upregulated in SFs from Tg197, and ex vivo cultured RA SFs secreted significantly more ATX protein than their wild-type counterparts.

### Development of In Vitro Assays to Test Properties of Ex-Vivo Primary Synovial Fibroblasts (Migration, Adhesion Proliferation and Apoptosis)

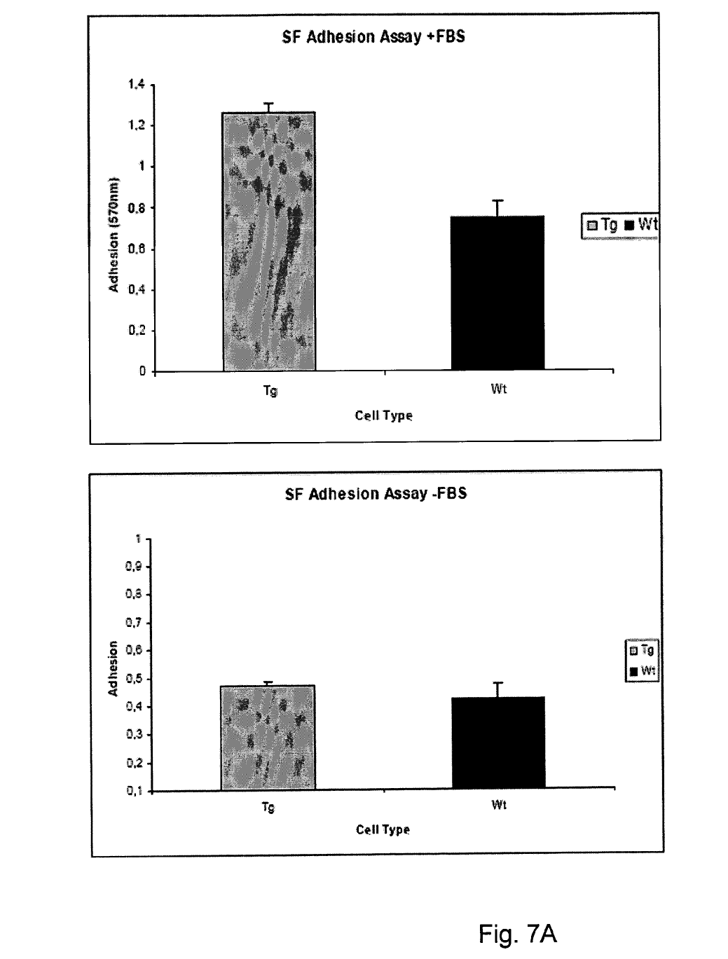

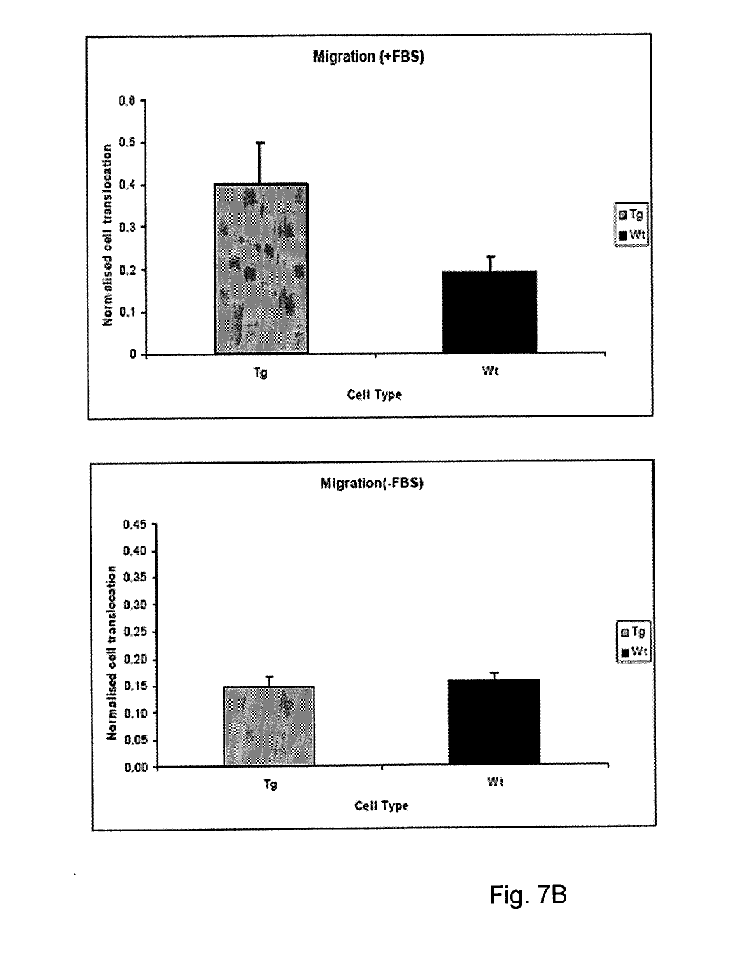

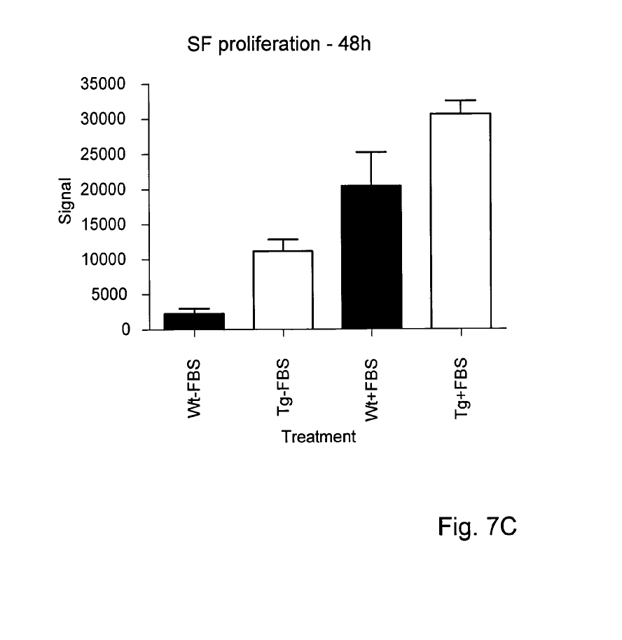

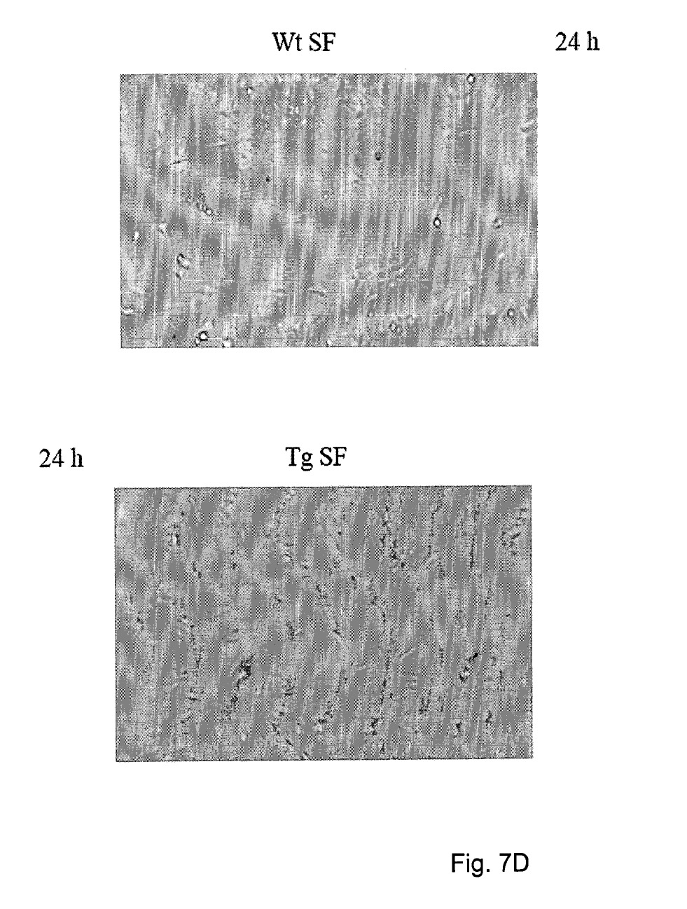

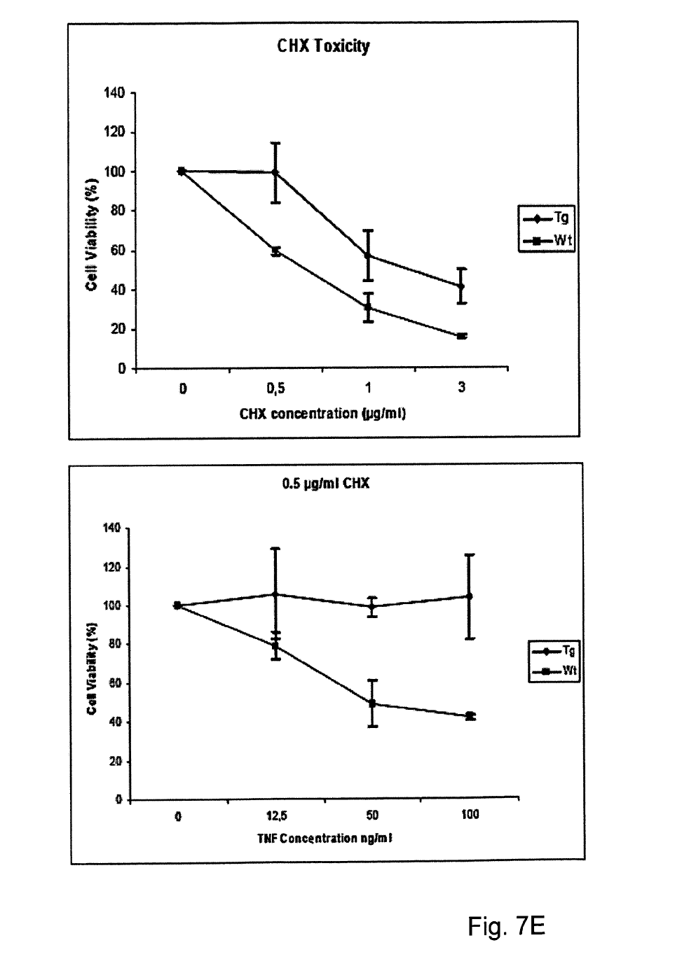

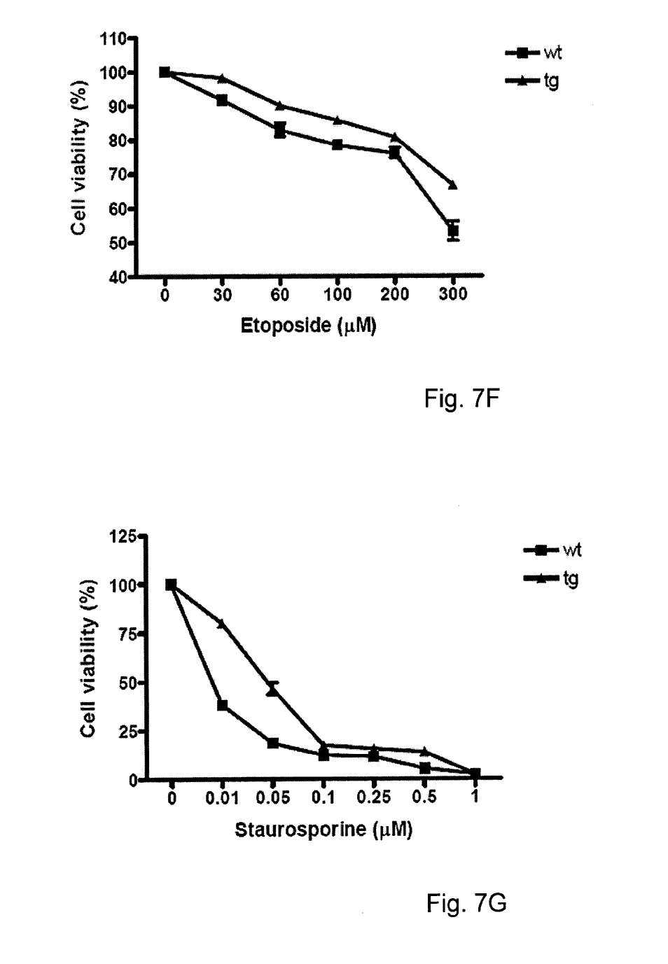

It is provided here that Tg SF's exhibit distinct phenotype compared to those of wild type mice. It was shown that Tg SFs have rearranged actin cytoskeleton and exhibit pronounced stress fibers. We also showed that Tg SF's adhere with increased affinity to the proteins of the ECM in vitro, while they possess greater motility compared with wild-type mice. This increased adhesion leads to a more elongated shape of the SF's. For this reason a number of in vitro assays were developed/standardised, including Adhesion, Migration, Proliferation, Wound Healing and Apoptosis (FIG. 7).

These in vitro assays demonstrate the distinct properties of Tg SFs, including increased adhesion, migration and apoptosis resistance compare to Wt SF's, while proliferation is increased. Targeting the expression cytoskeleton related genes (such as preferably Gelsolin, VASP, Cofilin 1 and/or Aquaporin 1) affects cytoskeleton arrangement. Knocking down the expression of such genes has an effect in the actin cytoskeleton machinery of the SF's and this is revealed by the above assays.

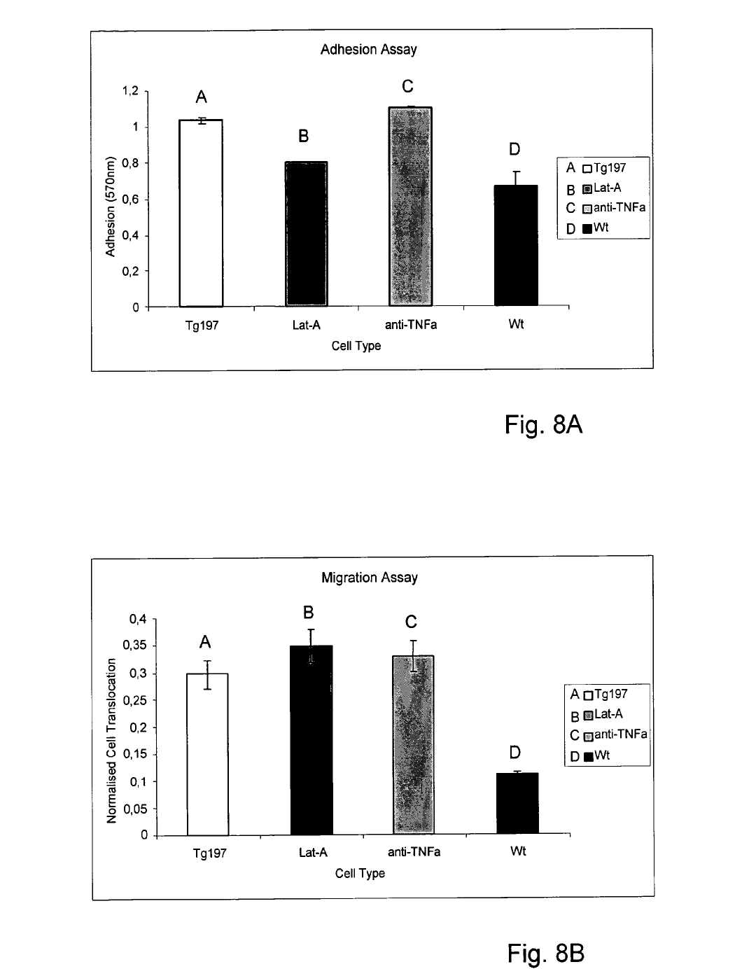

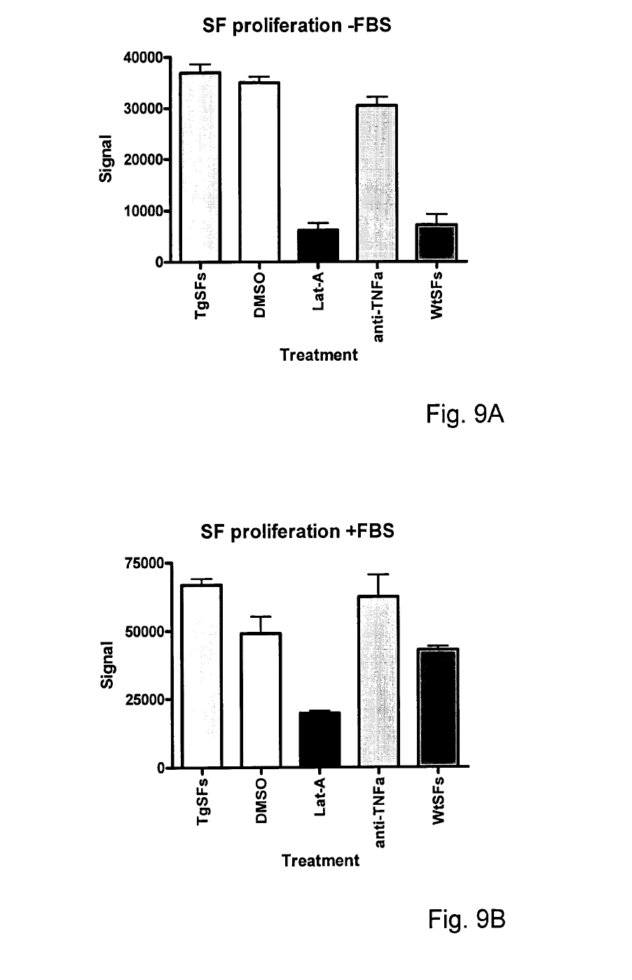

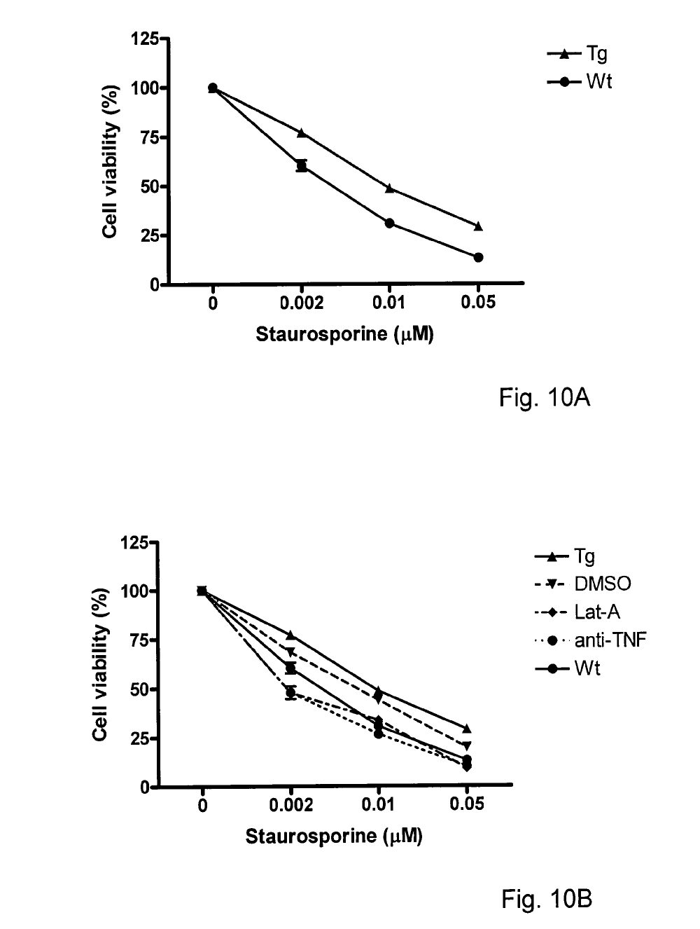

In vitro analysis on the effect of Latrunculin A (Lat-A), an inhibitor of F-actin polymerization (Giganti and Friederich, 2003), has indicated the potential of actin-drugs in altering the cytoskeleton properties of Tg197 synovial fibroblasts. In detail, treatment with Lat-A has resulted in decreased adhesion of TgSF's, while migration seems to be increased compare to Wt (FIG. 8) This is in keeping with previous findings of Lat-A treatment and myofibrils (Wang et al, 2005). At the same time Tg SF's treated with anti-TNF do not show altered adhesion/migration properties, suggesting that this effect is not TNF-driven. In addition, treatment with Lat-A seems to have a dramatic effect on the proliferation levels of TgSFs which decline lower even than the levels of WtSFs, while DMSO and TNFα do not have an effect on TgSFs rate of proliferation (FIG. 9). Most importantly, apoptosis assays using staurosporine as inducer of cell death revealed that Lat-A disruption of the cytoskeleton causes decreased resistance to apoptosis at the levels of the Wt SFs (FIG. 10). This result comes in concordance with previous findings that show that treatment with Cytochalasin D (an inhibitor of rapid actin polymerization) enhances apoptosis in HeLa cells, while phalloidin (an inducer of actin polymerization) does not affect apoptosis (Kulms et al, 2002).

### Validation of Selected Deregulated Genes (DEGs), on Human RA and OA Samples by Q-RT-PCR Analysis

The patent states that “analyzing by quantitative RT-PCR (Q-RT-PCR) DEGs selected from tables A, B and C may provide a good correlation with the absence or presence of inflammatory arthritis (IA). For this purpose a number of genes were selected (i.e. C3, CstB, Dusp1, LSP1, PITPNM, RAB14, SHIP2, TGFB1I4, TIMP1, TIMP3, TUBA1, C1QA, SHIP, LST1) based on the following criteria: a) DEGs that showed the greatest level of differential expression following validation in the Tg197 mouse model, by SQ-RT-PCR analysis; b) DEGs identified in the Tg197 mouse microarray data and are key players in the regulation of actin cytoskeleton and c) candidate genes of interest that were not identified by the microarray experiments (SHIP and SHIP2) but are known to be involved in the mechanism of actin cytoskeleton.

In order to carry out the above task a collaboration was established with Dr Wilson's lab at the University of Sheffield, UK, who agreed to provide us with sufficient amounts of cDNA from biopsies of RA and OA patients (Ethics no: SSREC 01/333; Project title “Regulation of cytokine genes in RA”. In detail, synovial tissue was obtained during joint replacement surgery from patients with RA (n=21), defined according to the 1987 revised criteria of the American College of Rheumatology (formerly, the American Rheumatism Association). Synovial tissue was also obtained from patients with osteoarthritis (OA) (n=12), who were undergoing knee joint replacement surgery. Patient's informed consent was obtained according to the national ethical rules, as these were implemented by the corresponding local ethic commission.

Quantitative real-time PCR was performed as previously described (Aidinis et al, 2005). Primers were chosen from either exons separated by large introns or exon boundaries and the PCR quality and specificity was verified by melting curve analysis and gel electrophoresis. Values were normalized to both B2M and L32. Human primer sequences and expected lengths were as follows (listed in the 5′ to 3′ direction and designated as s; sense and as; antisense): hC3 (s, GGCCTTTGTTCTCATCTCGCT; as, ATAGGATGTGGCCTCCACGTT, 274 bp), hCSTB (s, CCTGTGTTTAAGGCCGTGTCAT; as, GGAGAGATTGGAACACTCGCAG, 115 bp), hDUSP1 (s, ACAAGGCAGACATCAGCTCCTG; as, TAAGCAAGGCAGATGGTGGCT, 130 bp), hLSP1 (s, CAACAATTAAGAGCACCCCATC; as, TGGCAACAGGAAGCAACTTCT, 223 bp), hRAB14 (s, GCTTATTGTTCCTCGAAGCGAG; as, ATCCAAGCTTCCATCCTGAATG, 107 bp), hSHIP2 (s, GGCTCGGAGGAACCAAAACTAC; as, TGAGGTCCCCAAACCAGAAGA, 119 bp), hTGFB114 (s, TAGCTCCTCTGGTGCAAGTGTG; as, TCCACTTCTTCTCTGACCGCA, 102 bp), hLST1 (s, CGTGATGAGGAACTTGAGG; as, GCGATAACATTAGGTCAGGG, 114 bp).

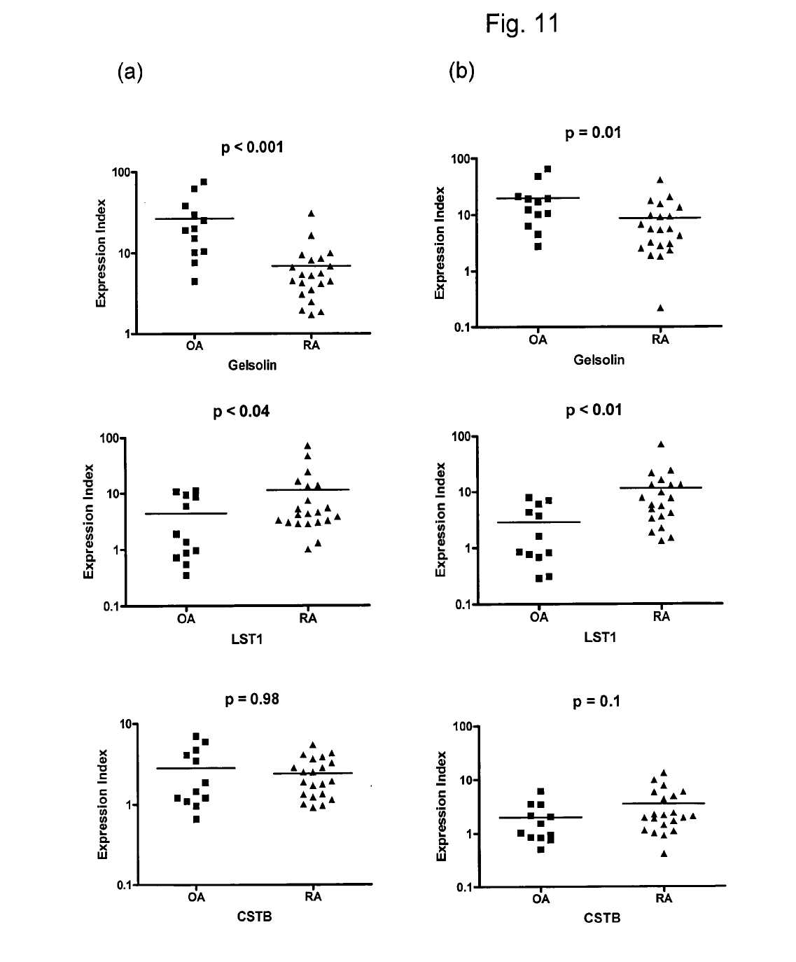

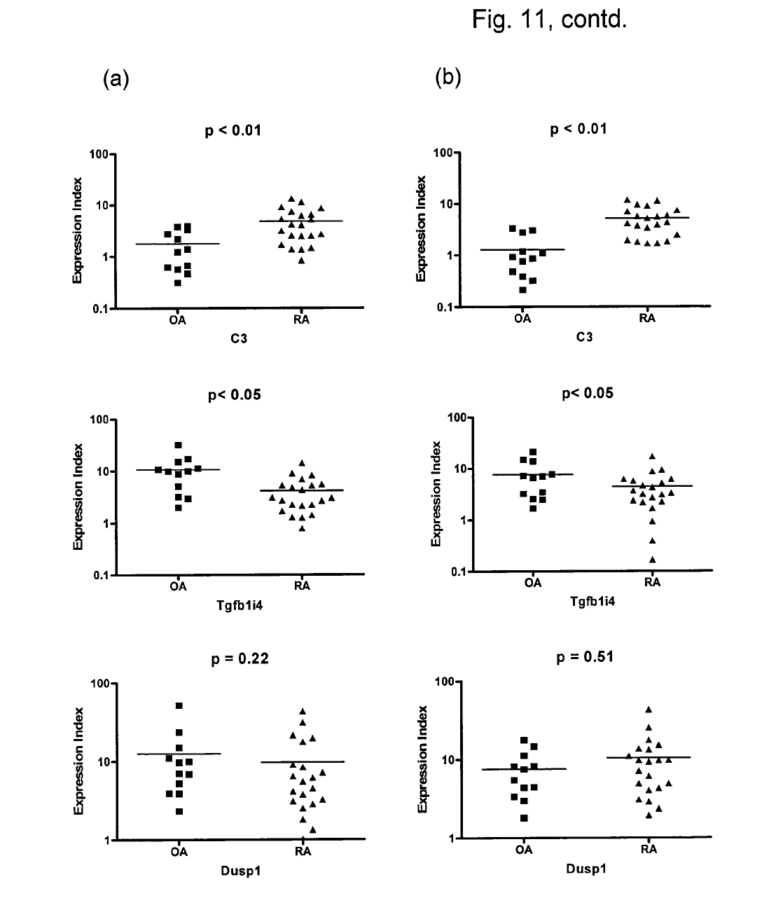

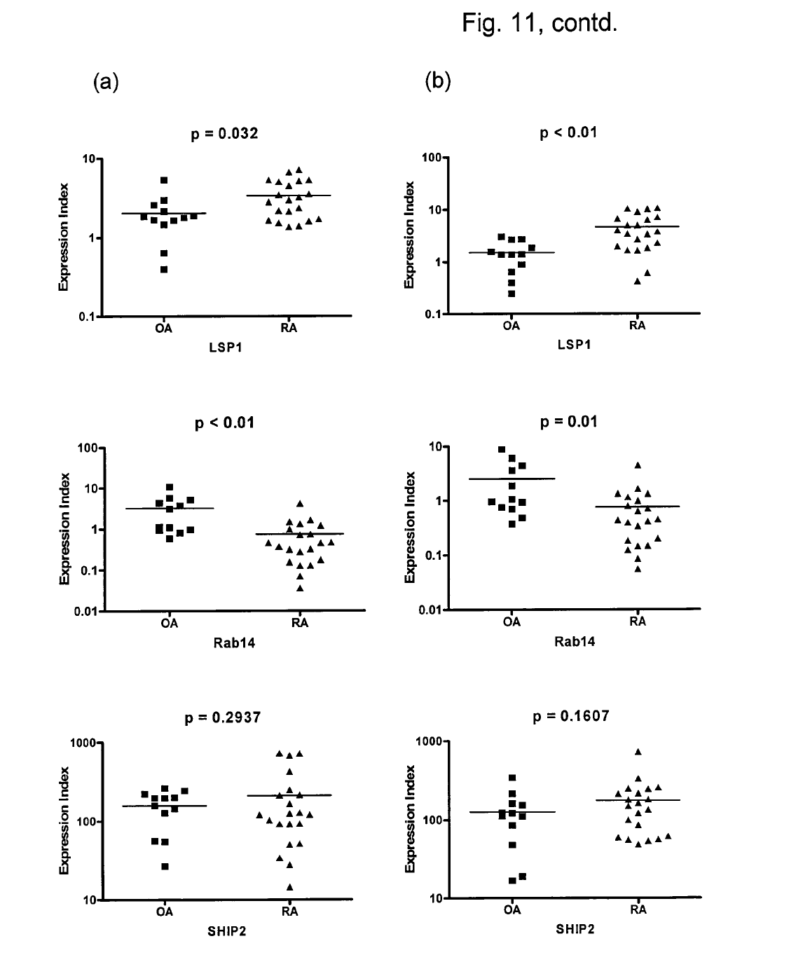

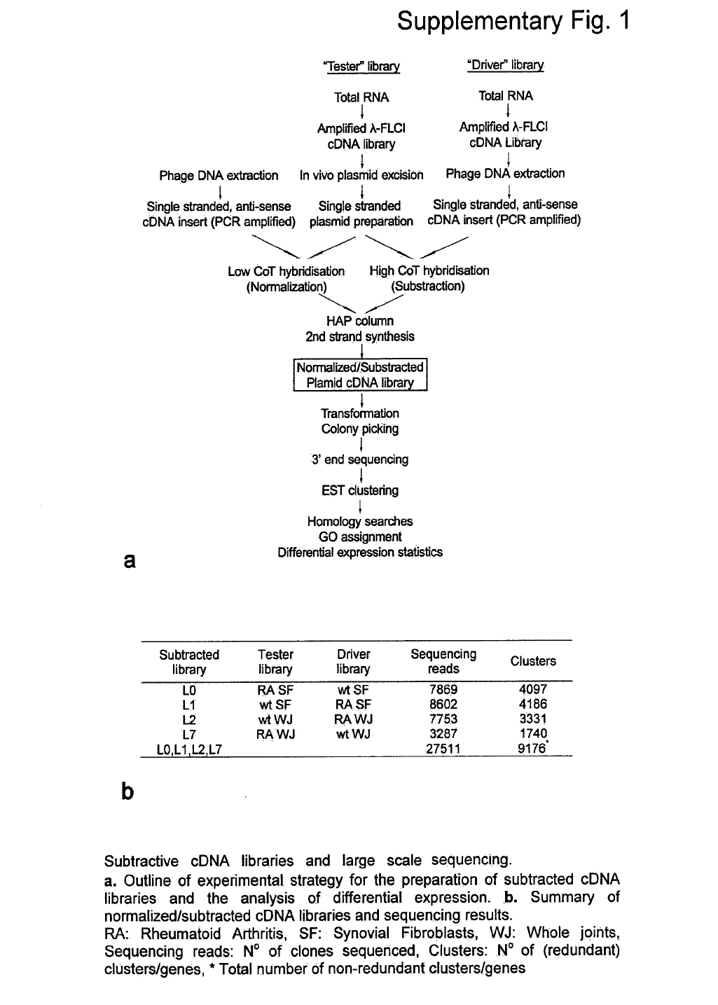

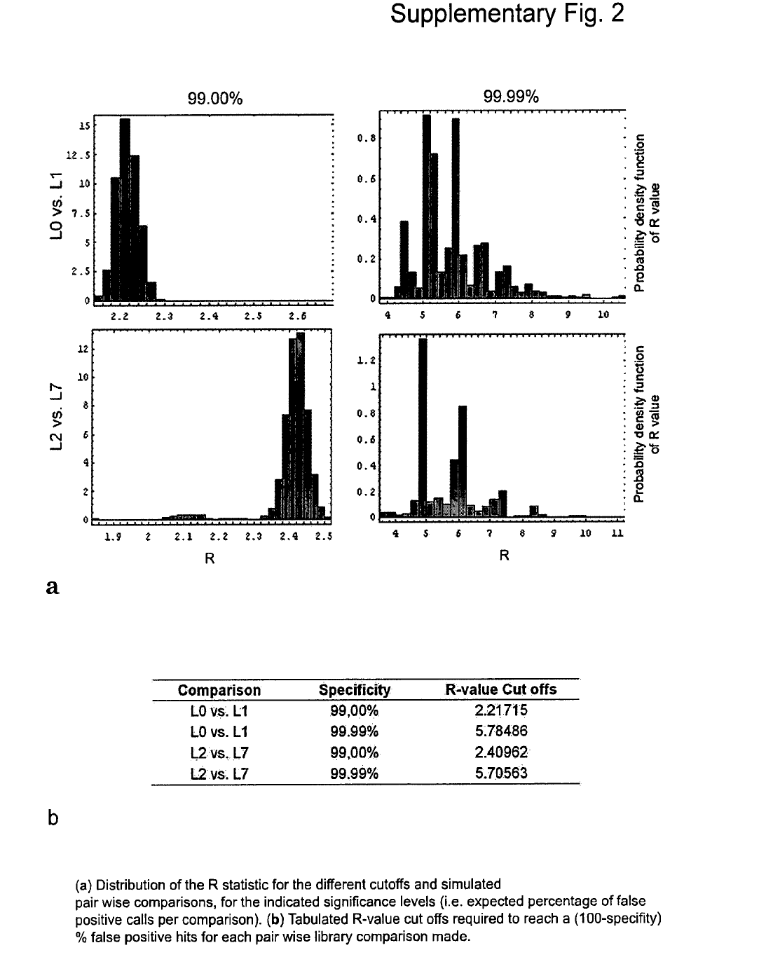

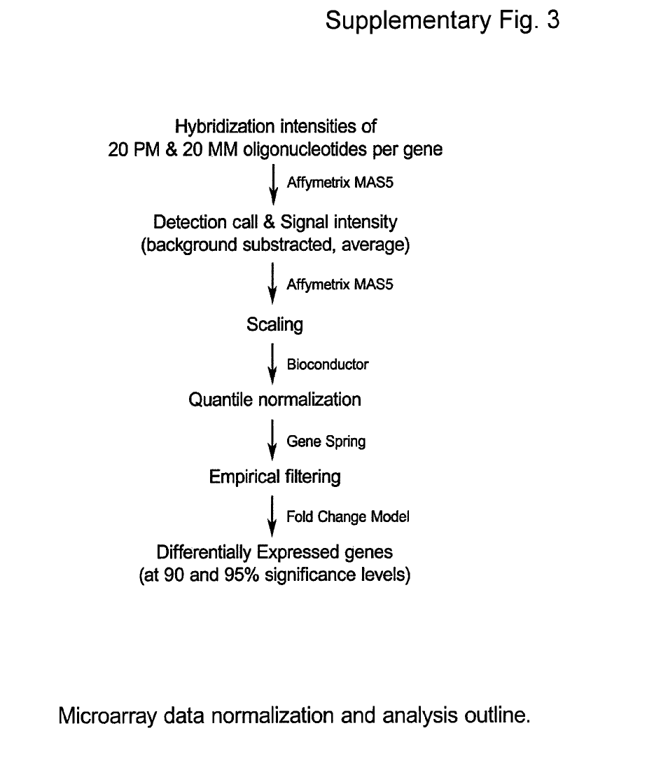

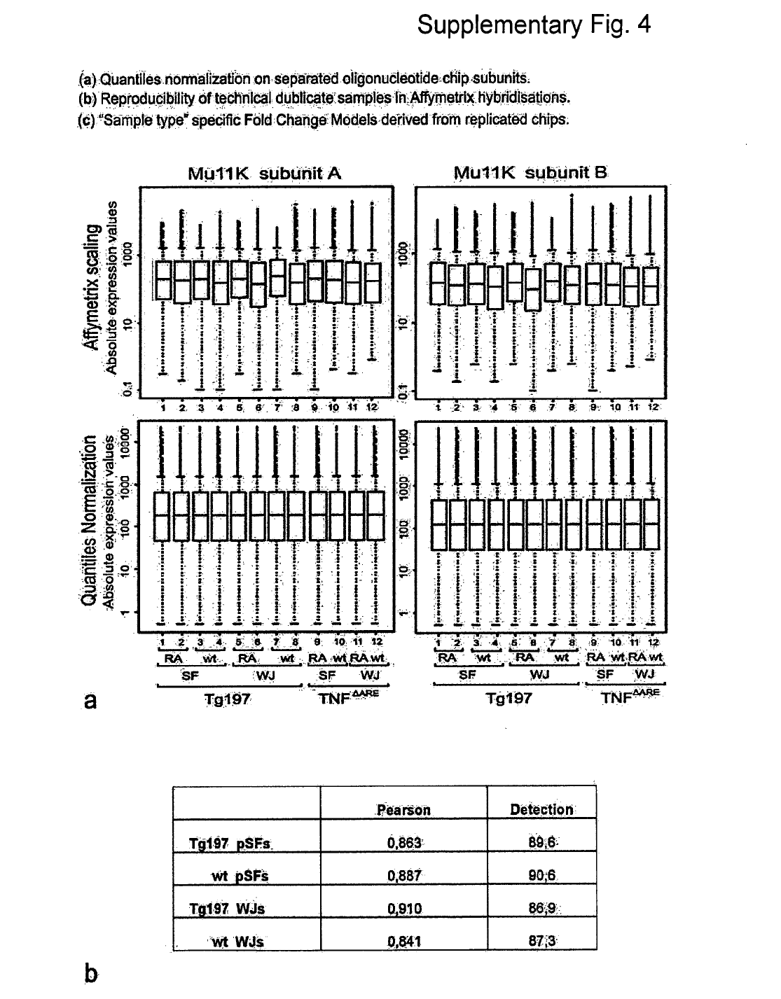

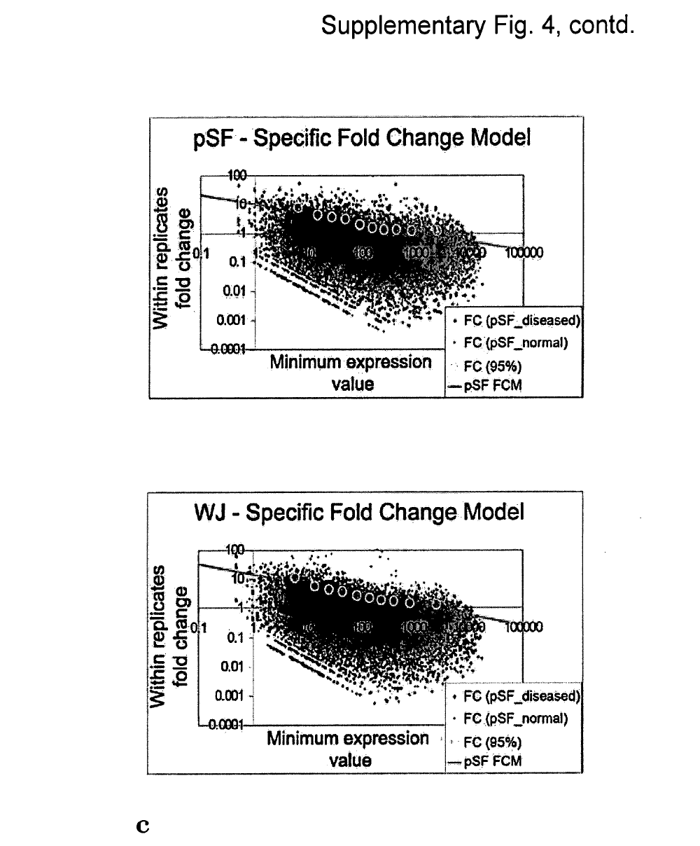

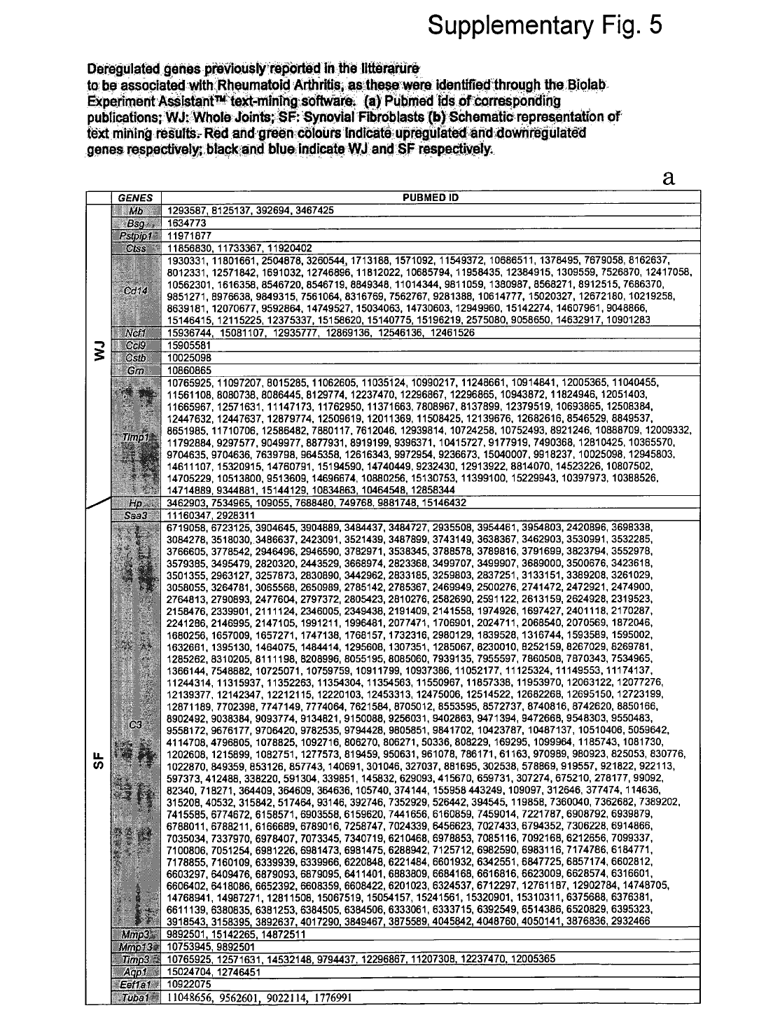

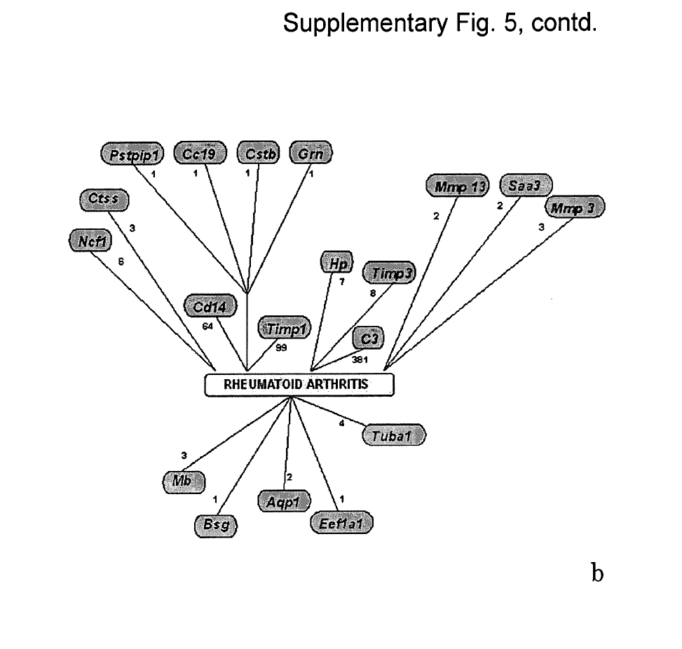

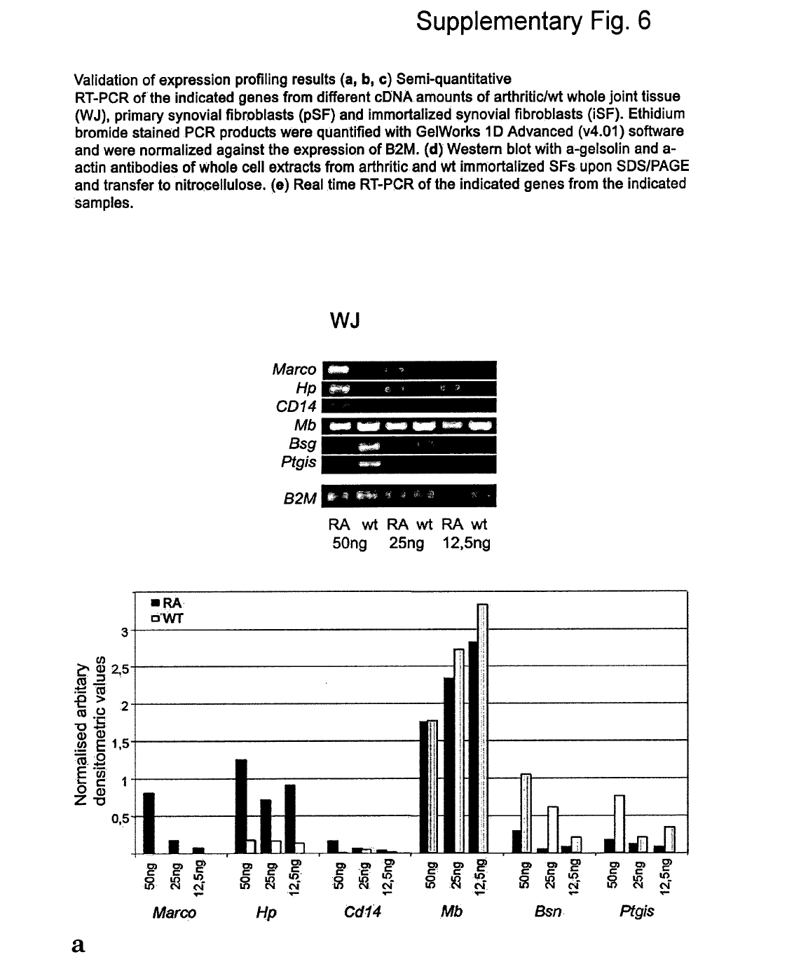

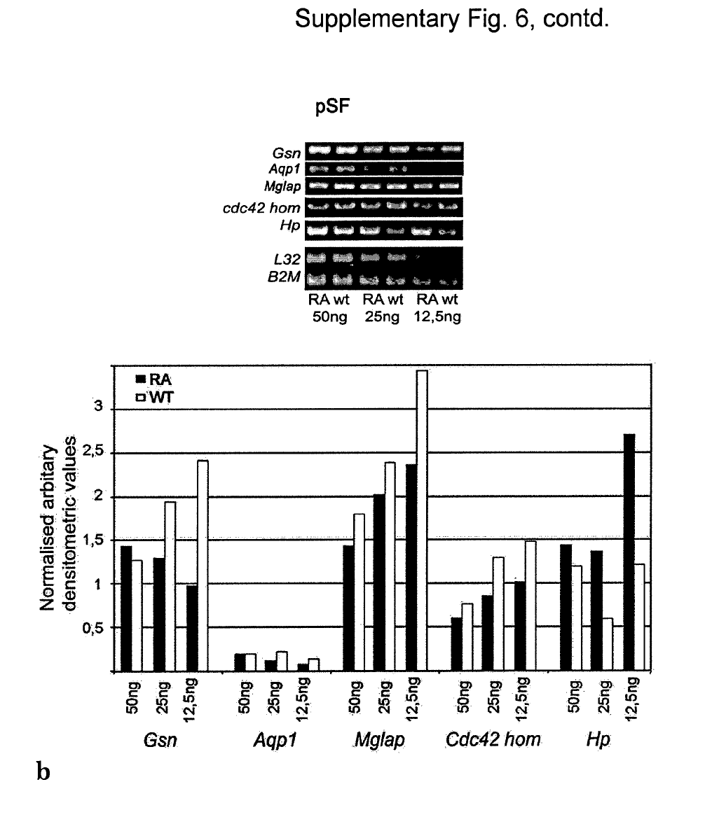

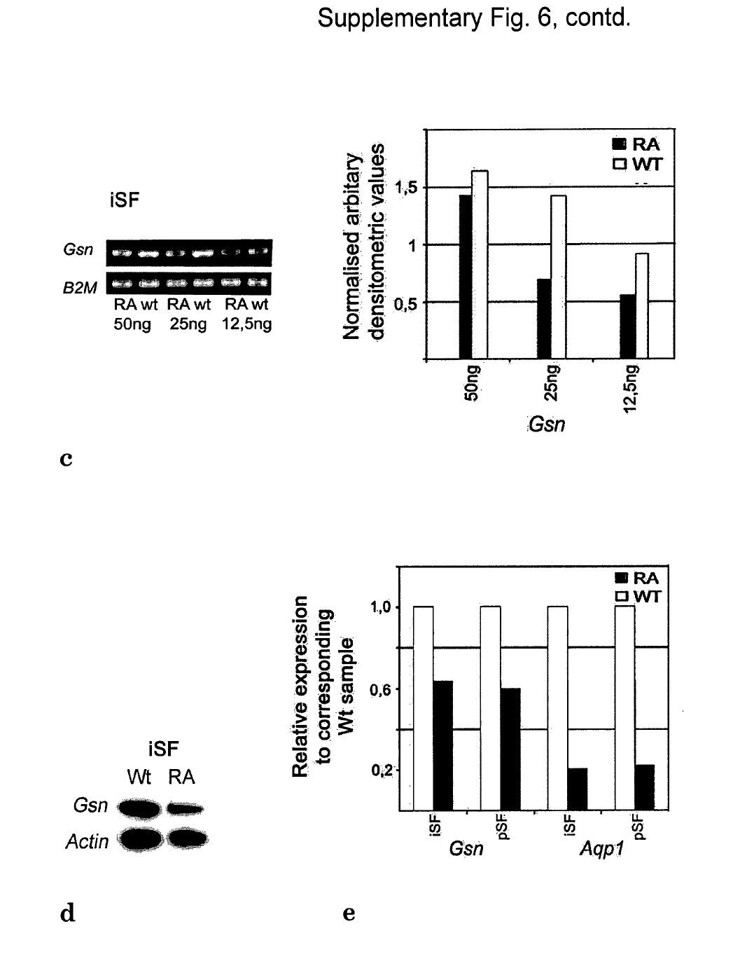

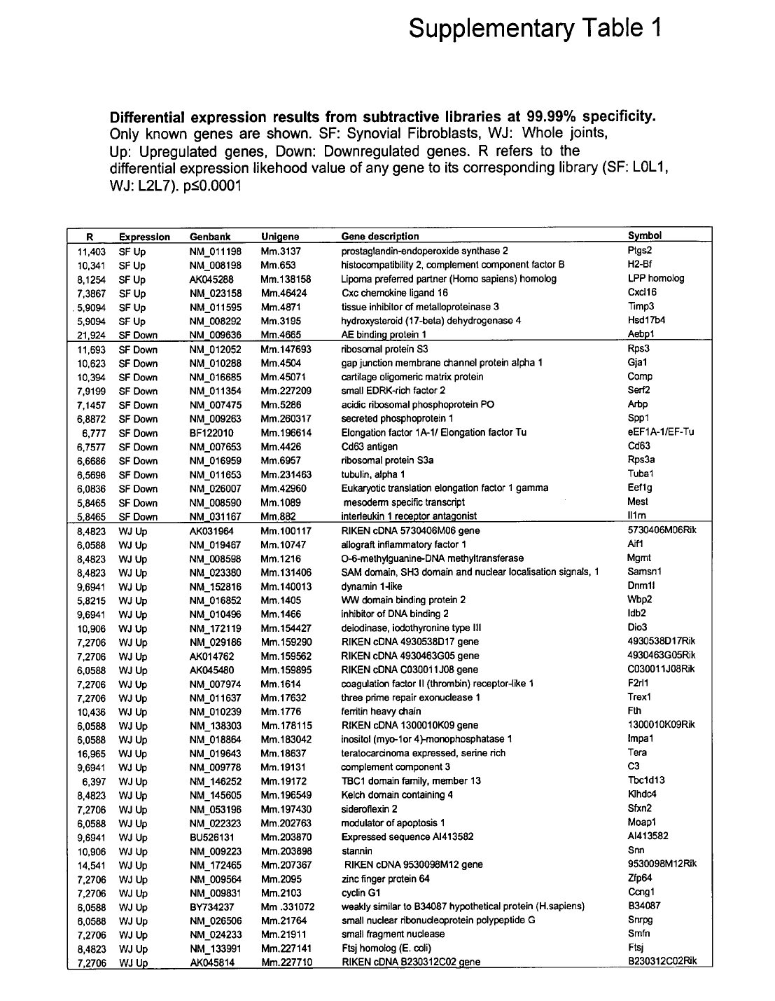

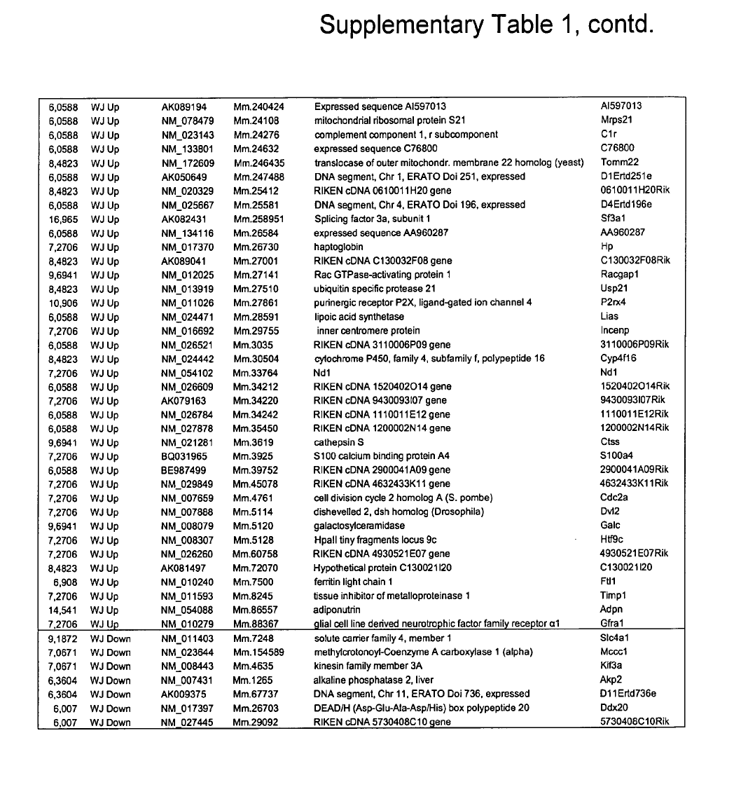

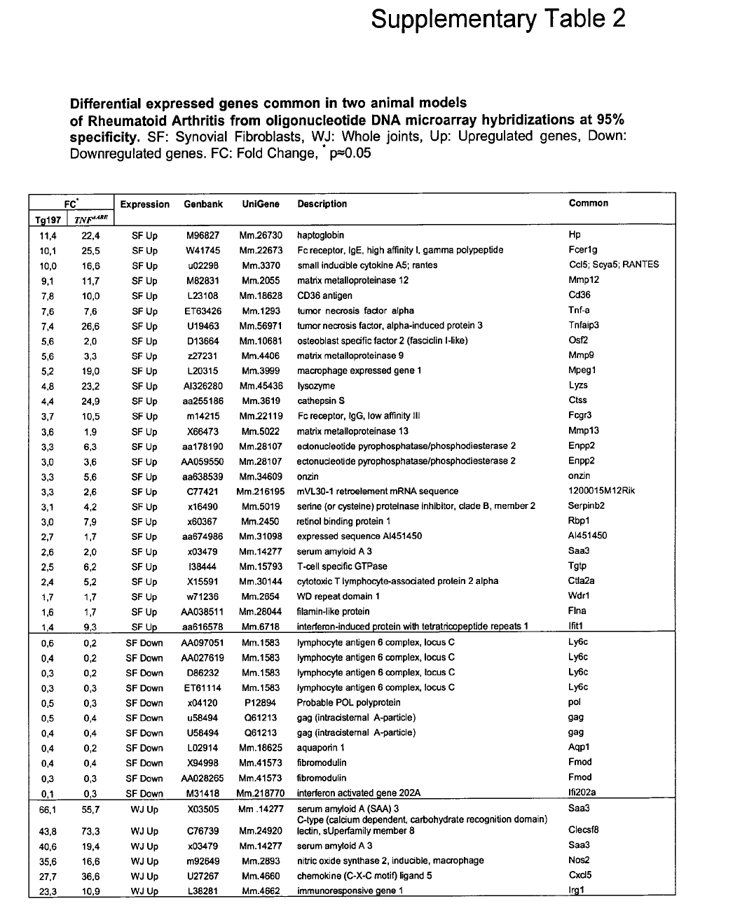

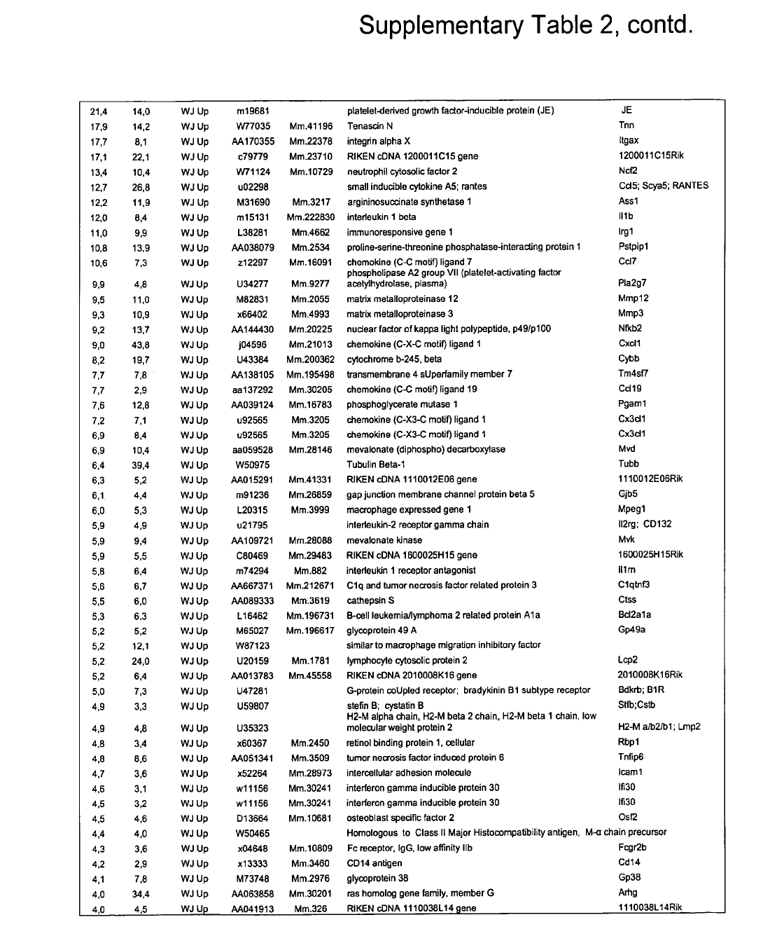

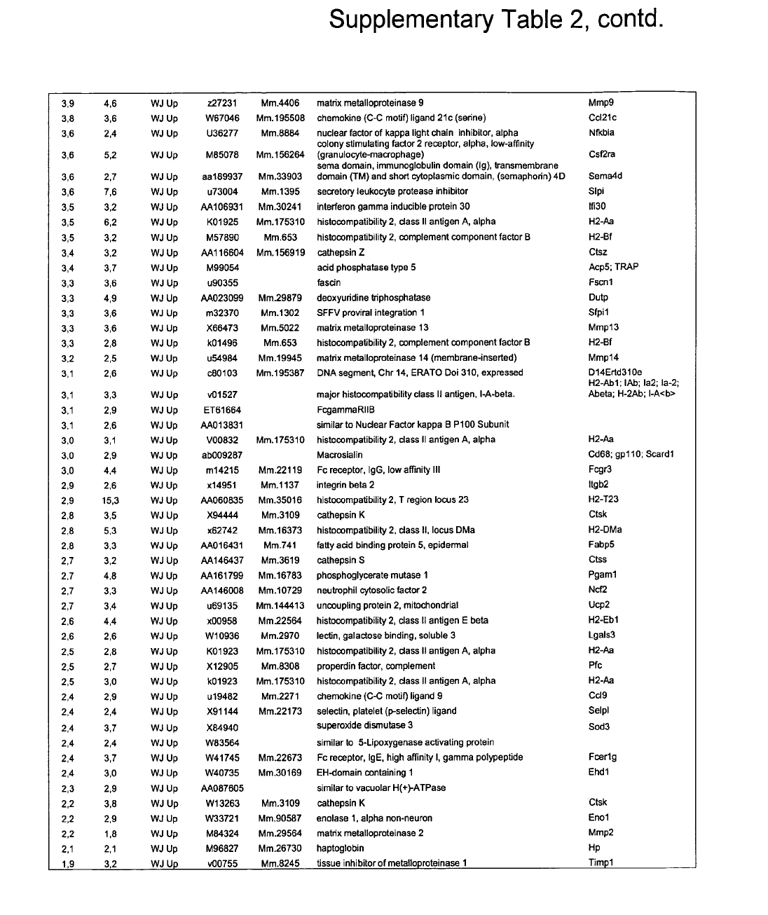

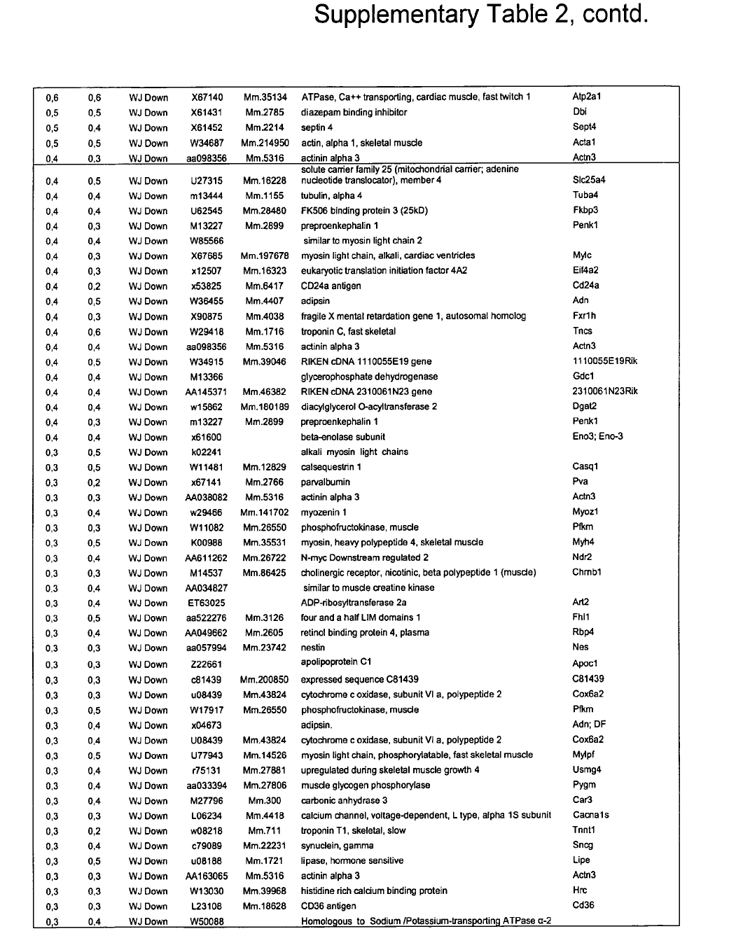

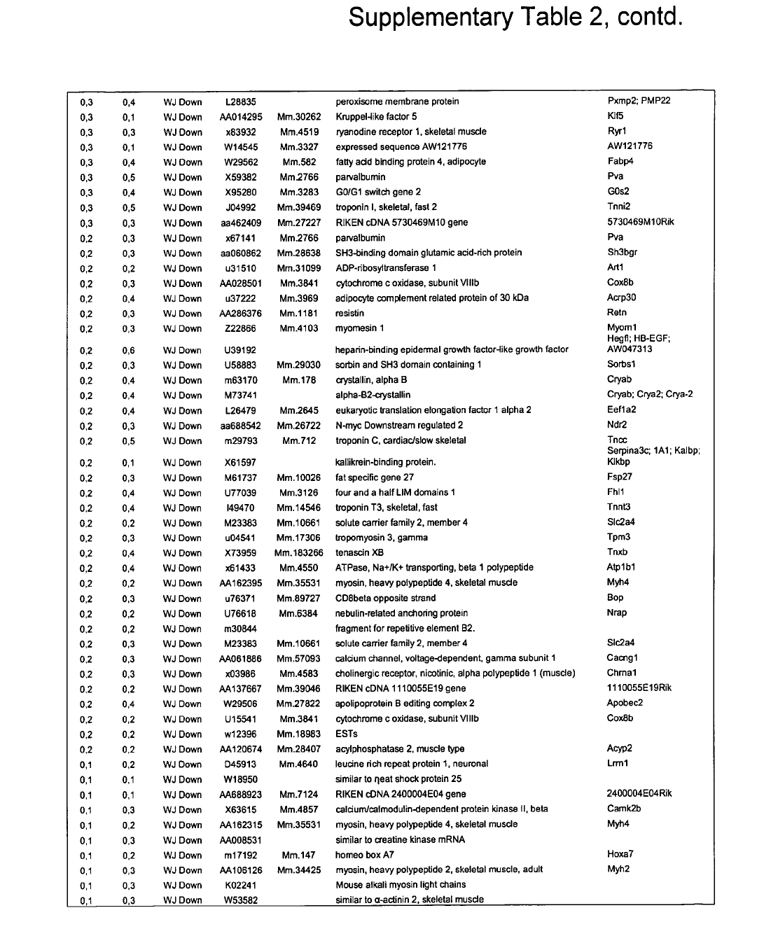

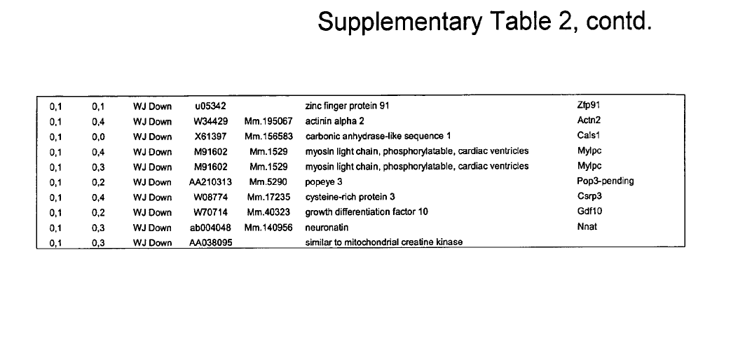

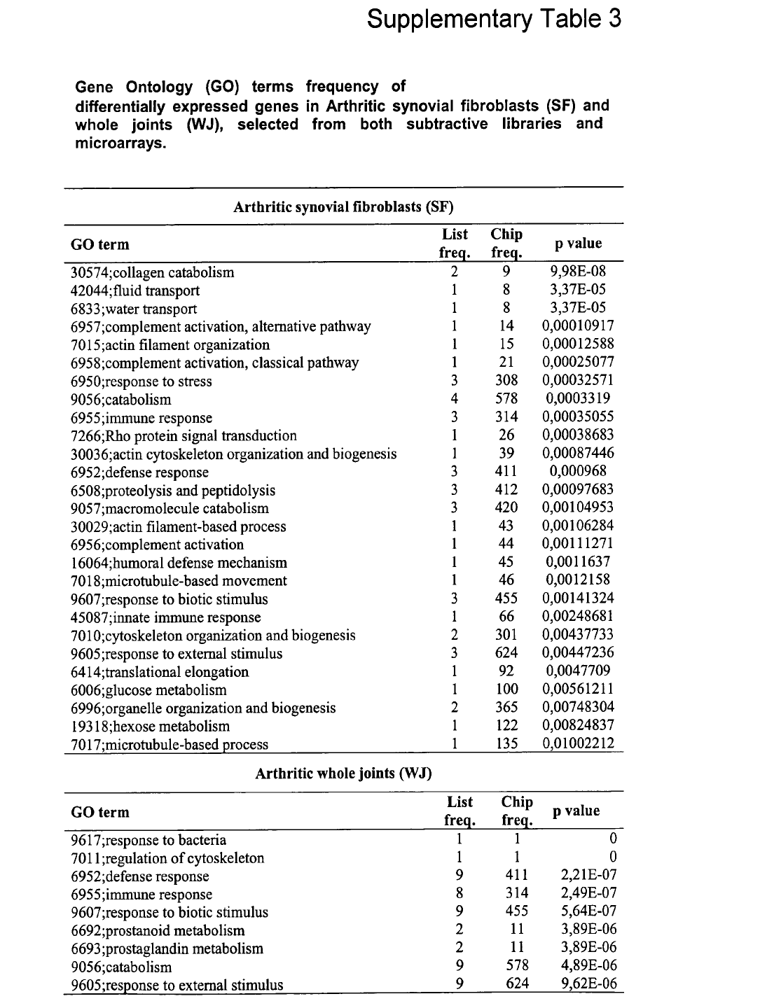

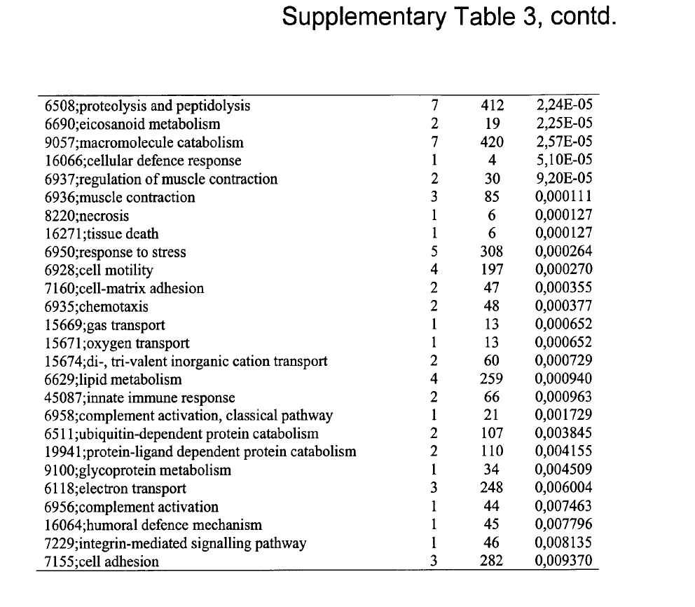

Of the 8 cytoskeleton-related DEGs studied, 5 showed the same pattern of deregulated expression in human samples and therefore merit further investigation (FIG. 11). Also another important conclusion is the significant association of down-regulation of gene expression of Gelsolin in RA patients, in another population. This strengthens the association of Gelsolin and actin-cytoskeleton re-arrangements in the pathogenesis of RA patients.

### REFERENCES TO EXAMPLE 2

1. Paavilainen V O, et al (2004) Regulation of cytoskeletal dynamics by actin-monomer-binding proteins. Trends Cell Biol 14:386-394.
2. Hotulainer P, et al (2005) Actin-depolymerizing factor and cofilin-1 play over-lapping roles in promoting rapid F-actin depolymerization in mammalian nonmuscle cells. Mol Biol Cell 16:649-64.
3. von Wichert G, et al (2003) Force-dependent integrin-cytoskeleton linkage formation requires down-regulation of focal complex dynamics by Shp2. Embo J 22:5023-35.
4. Song J, et al (2005) Autotaxin (lysoPLD/NPP2) protects fibroblasts from apoptosis through its enzymatic product, lysophosphatidic acid, utilizing albumin-bound substrate. Biochem Biophys Res Commun 337:967-75.
5. Svitkina T M, et al (1999) Arp2/3 complex and actin depolymerising factor/cofilin in dendritic organization and treadmilling of actin filament array in lamellipodia. J Cell Biol 145:1009-26.
6. Rohatgi R, et al (1999) The interaction between N-WASP and the Arp2/3 complex links Cdc42-dependent signals to actin assembly. Cell 97:221-31.
7. Gallego M D, et al (2005) WIP and WASP play complementary roles in T cell homing and chemotaxis to SDF-1a. Inter Immunol 18:221-32.
8. Machesky L M, et al (1999) Scar, a WASp-related protein, activates nucleation of actin filaments by the Arp2/3 complex. Proc Natl Acad Sci USA 96:3739-44.
9. Gautreau A, et al (2002) ERM proteins and NF2 tumor suppressor: the Yin and Yang of cortical actin organization and cell growth signalling. Curr Opin Cell Biol 14:104-19.
10. Hoffman L N, et al (2006) J Cell Biol Genetic ablation of zyxin causes Mena/VASP mislocalization, increased motility, and deficits in actin remodeling 172:771-82.
11. Weaver A M, et al (2001) Cortactin promotes and stabilizes Arp2/3-induced actin filament network formation. Curr Biol 11:370-4.
12. Schluter K, et al (1997) Profilins as regulators of actin dynamics. Biochim Biophys Acta 1359:97-109.
13. Carlier M, et al, (1993) Modulation of the interaction between G-actin and thymosin beta 4 by the ATP/ADP ratio: possible implication in the regulation of actin dynamics. Proc Natl Acad Sci USA 90:5034-8
14. Rohatgi R, et al (2001) Nck and phosphatidylinositol 4,5-bisphosphate synergistically activate actin polymerization through the N-WASP-Arp2/3 pathway. J Biol Chem 276:26448.
15. Foger N, et al (2006) Requirement for coronin 1 in T lymphocyte trafficking and cellular homeostasis. Science 313:839-42.
16. Nyakern-Meazza M, et al (2002) Tropomyosin and gelsolin cooperate in controlling the microfilament system. J Biol Chem 277:28774-9.
17. Kordowska J, et al (2006) Phosphorylated 1-caldesmon is involved in disassembly of actin stress fibers and postmitotic spreading. Exp Cell Res 312:95-110.
18. Cooper J A and Schafer D A (2000) Control of actin assembly and disassembly at filament ends. Curr Opin Cell Biol 12:97-103.
19. Fujisawa T, et al (2001) The shapes and sizes of two domains of tropomodulin, the P-end-capping protein of actin-tropomyosin. FEBS Lett 498:67-71.
20. Rodal A A, et al (1999) Aip1p interacts with cofilin to disassemble actin filaments. J Cell Biol 145:1251-64.
21. Moseley J B, et al (2006) Twinfilin is an actin-filament-severing protein and promotes rapid turnover of actin structures in vivo J Cell Sci 119:1547-57.
22. DeMali K A, et al (2002) Recruitment of the Arp2/3 complex to vinculin: coupling membrane protrusion to matrix adhesion. J Cell Biol 159:881-91.
23. Ohta Y, et al (2006) FilGAP, a Rho- and ROCK-regulated GAP for Rac binds filamin A to control actin remodelling. Nat Cell Biol 8:803-14.
24. Pawlak G and Helfman D M (2002) MEK mediates v-Src-induced disruption of the actin cytoskeleton via inactivation of the Rho-ROCK-LIM kinase pathway. Biol Chem 277:26927-33.
25. Schratt G. et al (2002) Serum response factor is crucial for actin cytoskeletal organization and focal adhesion assembly in embryonic stem cells. J Cell Biol 156:737-50.
26. Lamarche N. et al (1996) Rac and cdc42 induce actin polymerization and G1 cell cycle progression independently of p65PAK and the JNK/SAPK MAP kinase cascade. Cell 87:519-29.
27. Trentham D E, et al (1977). Autoimmunity to type II collagen an experimental model of arthritis. J Exp Mod 146:857-68.
28. Elson C J, et al (1992). Mediators of joint swelling and damage in rheumatoid arthritis and pristane induced arthritis. Autoimmunity 13:327-31.
29. Glant T, et al (2003). Proteoglycan-induced arthritis: immune regulation, cellular mechanisms, and genetics. Crit. Rev Immunol 23:199-250.
30. van den Broek M F, et al (1988) Streptococcal cell wall-induced arthritis and flare-up reaction in mice induced by homologous or heterologous cell walls. Am Pathol 133:139-49.
31. van Lent, et al (2003) Expression of the dendritic cell-associated C-type lectin DC-SIGN by inflammatory matrix metalloproteinase-producing macrophages in rheumatoid arthritis synovium and interaction with intercellular adhesion molecule 3-positive T cells. Arthritis Rheum 48:360-9.
32. Schalkwijk et al, (1985) Cationization of catalase, peroxidase, and superoxide dismutase. Effect of improved intraarticular retention on experimental arthritis in mice. J Clin Invest 76:198-205.
33. Hultqvist M, et al (2004) Enhanced autoimmunity, arthritis and encephalomyelitis in mice with a reduced oxidative burst due to a mutation in the Ncf1 gene. Proc Natl Acad Sci USA 101:12646-51.
34. Sakaguchi N, et al (2003) Altered thymic T-cell selection due to a mutation of the ZAP-70 gene causes autoimmune arthritis in mice. Nature 426:454-60.
35. Hang L, et al (1982) A spontaneous rheumatoid arthritis-like disease in MRL/1 mice. J Exp Med 155:1690-1701.
36. Kouskoff V, et al (1996) Organ-specific disease provoked by systemic autoimmunity. Cell 87:811-22.
37. Iwakura Y, et al (1991) Induction of inflammatory arthropathy resembling rheumatoid arthritis in mice transgenic for HTLV-I. Science 253:1026-28.
38. Niki Y, et al (2001) Macrophage- and neutrophil-dominant arthritis in human IL-1 alpha transgenic mice. J Clin Invest 107:1127-35.
39. Horai R, et al (2000) Development of chronic inflammatory arthropathy resembling rheumatoid arthritis in interleukin 1 receptor antagonist-deficient mice. J Exp Med 191:313-20.
40. Atsumi T, et al (2002) A point mutation in of Tyr-759 in interleukin 6 family cytokine receptor subunit gp130 causes autoimmune arthritis. J Exp Med 196:979-90.
41. Keffer J, et al (1991) Transgenic mice expressing human necrosis factor: A predictive genetic model of arthritis. EMBO J. 10:4025-31.
42. Kontoyiannis D, et al (1999) Impaired On/off regulation of TNF biosynthesis in mice lacking TNF AU-rich elements: Implications for joint and gut-associated immunopathologies. Immunity 10:387-98.
43. Taylor G A, et al (1996) A pathogenetic role of TNF alpha in the syndrome of cachexia, arthritis and autoimmunity resulting from tristetraprolin (TTP) deficiency. Immunity 4:445-54.
44. Amano T, et al (2006) Synoviolin/Hrdl, an E3 ubiquitin ligase, as a novel pathogenic factor for arthropathy. Genes Dev 17:2436-49.
45. Hauser W, et al (1999) Megakaryote hyperplasia and enhanced agonist-induced platelet activation in vasodilator-stimulated phosphoprotein knockout mice. Proc. Natl. Acad. Sci. USA. 96:8120-5.
46. Gurniak C B, et al (2005) The actin depolymerising factor n-cofilin is essential for neural tube morphogenesis and neural crest cell migration. Develop Biol 278:231-41.
47. Aidinis V, et al (2005) Cytoskeletal rearrangements in synovial fibroblasts as a novel pathophysiological determinant of modeled rheumatoid arthritis. Plos Genet. 1:e48.
48. Raghunathan A, et al (2001) Functional analysis of B144/LST1: A gene in the tumor necrosis factor cluster that induces formation of long filopodia in eukaryotic cells. Exp Cell Res 268:230-44.
49. Chrostek A, et al (2006) RacI is crucial for hair follicle integrity but is not essential for maintenance of the epidermis. Mol Cell Biol 26:6957-70.
50. Wiebel F F, et al (2002) Generation of mice carrying conditional knockout alleles for the transcription factor SRF. Genesis 32:124-6.
51. Wang J, et al (2005) Differential effects of Latrunculin-A on myofibrils in cultures of skeletal muscle cells: Insights into mechanisms of myofibrillogenesis. Cell Motility Cytoskeleton 62:35-47.
52. Kulms D, et al (2002) Apoptosis induced by disruption of the actin cytoskeleton is mediated via activation of CD95 (Fas/APO-1). Cell Death Differentiation 9:598-608.
53. Xia X G, et al. (2006) Pol II-expressed shRNA knocks down Sod2 gene expression and causes phenotypes of the gene knockout in mice. Plos Genet. 2:e10.
54. Song, A. (2003) RNA interference targeting Fas protects mice from fulminant hepatitis. Nat Med 9:347
55. Soutschek J, et al (2004) Therapeutic silencing of an endogenous gene by systemic administration of modified siRNAs. Nature 432:173.
56. Lechman E R, et al (2003) The contralateral effect conferred by intra-articular adenovirus-mediated gene transfer of viral IL-10 is specific to the immunizing antigen. Gene Ther 24:2029-35.
57. Harper S Q, et al. (2005) RNA interference improves motor rand neuropathological abnormalities in a Huntington's disease mouse model. PNAS 102:5820
58. Makarov S S, et al (1996) Suppression of experimental arthritis by gene transfer of interleukin 1 receptor antagonist cDNA. Proc Natl Acad Sci USA. 93:402.
59. Aidinis V, et al (2003) Functional analysis of an arthritogenic synovial fibroblast. Arthritis Res Ther 5:R140-57.
60. Spector I, et al (1989) Latrunculins: novel marine macrolides that disrupt microfilament organization and affect cell growth: I. Comparison with Cytochalasin D. Cell Motil Cytoskeleton 13:127-44.
61. Giganti A and Friederich E (2003) The actin cytoskeleton as a therapeutic target: state of the art and future directions. Prog Cell Cycle Res 5:511-25.
62. Tomasek J J, et al (2002) Myofibroblasts and mechano-regulation of connective tissue remodeling. Nat Rev Mol Cell Biol 3:349-63.

TABLE 1
Summary results of subtractive libraries and microarray hybridizations
a. Subtractive libraries

| | Total genes | | Unigene | | FANTOM | | novel | |
|---|---|---|---|---|---|---|---|---|
| Sample | 99% | 99.99% | 99% | 99.99% | 99% | 99.99% | 99% | 99.99% |
| SF | 288 | 23 | 264* | 20** | 14 | 1 | 10 | 2 |
| WJ | 653 | 95 | 552* | 77** | 30 | 3 | 71 | 15 |

b. Oligonucleotide microarray hybridizations

| | | |
|---|---|---|
| Tg197 | TNF^{ΔARE} | Common |

| Sample | 90% | 95% | 90% | 95% | 90% | 95% |
|---|---|---|---|---|---|---|
| SF | 277* | 98 | 752 | 363 | 95 | 38*** |
| WJ | 830* | 498 | 1226 | 636 | 413 | 227*** |

a. Summary results of subtractive libraries. Total genes: N^{o} of statistically significant deregulated clusters/genes; Unigene: N^{o} of total genes with a Unigene cluster ID; FANTOM: N^{o} of total genes with a FANTOM accession N^{o}.
b. Summary results of oligonucleotide microarray hybridizations. Numbers correspond to Affymetrix probesets.
a and b. SF: Synovial Fibroblasts, WJ: Whole joints; Percentages indicate statistical specificities.
*Genes used for the cross-platform comparison presented in Table 2;
**Genes presented in Supplementary Table 1;
***Probesets corresponding to the genes presented in Supplementary Table 2;

TABLE 2
Differentially expressed genes in arthritic Whole Joints (WJ) or Synovial fibroblasts (SF) commonly
identified by subtractive libraries and oligonucleotide DNA microarray hybridizations.

| | | | | | | | | | | | m SQ- | m Q- | h Q- | |
|---|---|---|---|---|---|---|---|---|---|---|---|---|---|---|
| | | | | | | | | | Liter- | | RT- | RT- | RT- | |
| Rank | R* | | FC* | | Genbank | Name | Description | TNF^{ΔARE+/-} | ature | QTL | PCR | PCR | PCR | |
| 1 | 5.2 | (4) | 11.4 | (1) | M96827 | Hp | haptoglobin | + | + | | + | | | SF |
| 2 | 2.9 | (2) | 2.7 | (4) | aa674986 | AI451450 | expressed sequence | + | | | | | | up |
| | | | | | | | AI451450 | | | | | | | |
| 3 | 2.8 | (1) | 2.6 | (5) | x66402 | Mmp3 | matrix metallo- | − | + | | | | | |
| | | | | | | | proteinase 3 | | | | | | | |
| 4 | 4.4 | (5) | 3.2 | (3) | k02782 | C3 | complement | + | + | | | | | |
| | | | | | | | component 3 | | | | | | | |
| 5 | 3.3 | (8) | 3.6 | (2) | X66473 | Mmp13 | matrix metallo- | + | + | | | | | |
| | | | | | | | proteinase 13 | | | | | | | |
| 6 | 5.9 | (3) | 1.4 | (7) | z30970 | Timp3 | tissue inhibitor of | + | + | | | | | |
| | | | | | | | metalloproteinase 3 | | | | | | | |
| 7 | 3.7 | (7) | 2.6 | (6) | x03479 | Saa3 | serum amyloid A 3 | + | + | | | | | |
| 8 | 3.7 | (6) | 1.3 | (8) | W41762 | Rab14 | RAB14. (GTPase) | + | | + | +/− | − | | |
| | | | | | | | member RAS | | | | | | | |
| | | | | | | | oncogene family | | | | | | | |
| 1 | 6.7 | (1) | 0.3 | (1) | W44091 | Eef1a1 | elongation factor | + | + | | | | | SF |
| | | | | | | | 1-alpha | | | | | | | down |
| 2 | 3.3 | (4) | 0.3 | (2) | AA096992 | cdc42 | cell division | + | | | + | | − | |
| | | | | | | hom | cycle 42 homolog | | | | | | | |
| 3 | 6.6 | (2) | 0.7 | (5) | M13445 | Tuba1 | tubulin. alpha 1 | − | + | | +/− | | | |
| 4 | 2.6 | (5) | 0.5 | (4) | D00613 | Mglap | matrix gamma- | + | | | + | | | |
| | | | | | | | carboxyglutamate | | | | | | | |
| | | | | | | | (gla) protein | | | | | | | |
| 5 | 2.3 | (7) | 0.4 | (3) | L02914 | Aqp1 | aquaporin 1 | + | + | + | + | + | + | |
| | | | | | | | similar to | | | | | | | |
| | | | | | | | -glyceraldehyde- | | | | | | | |
| | | | | | | | 3-phosphate | | | | | | | |
| 6 | 4.7 | (3) | 0.8 | (7) | M32599 | Gapdh | dehydrogenase | − | | | | | | |
| | | | | | | hom | | | | | | | | |
| 7 | 2.6 | (6) | 0.7 | (6) | J04953 | Gsn | Gelsolin | − | | + | + | + | +* | |
| 1 | 9.7 | (1) | 5.5/2.7 | (5) | AA089333 | Ctss | cathepsin S | + | + | + | | | | WJ |
| | | | | | | | proline-serine- | | | | | | | up |
| | | | | | | | threonine | | | | | | | |
| | | | | | | | phosphatase- | | | | | | | |
| | | | | | | | interacting | | | | | | | |
| 2 | 4.8 | (5) | 10.8 | (2) | AA038079 | Pstpip1 | protein 1 | + | + | | | | | |
| 3 | 3.6 | (9) | 17.7 | (1) | AA170355 | Itgax | integrin alpha X | + | | | | | | |
| 4 | 4.8 | (6) | 4.2 | (4) | x13333 | Cd14 | CD14 antigen | + | + | | + | | + | |
| 5 | 7.3 | (2) | 2.1 | (15) | M96827 | Hp | haptoglobin | + | + | | + | | | |
| 6 | 2.4 | (15) | 7.3 | (3) | U18424 | Marco | macrophage | − | | | + | | + | |
| | | | | | | | receptor with | | | | | | | |
| | | | | | | | collagenous | | | | | | | |
| | | | | | | | structure | | | | | | | |
| 7 | 3.7 | (8) | 2.7 | (11) | L11455 | Ncf1 | neutrophil | + | + | + | | | | |
| | | | | | | | cytosolic | | | | | | | |
| | | | | | | | factor 1 | | | | | | | |
| 8 | 3.6 | (10) | 3.4 | (10) | U72643 | Lst1 | leukocyte | + | | | | | | |
| | | | | | | | specific | | | | | | | |
| | | | | | | | transcript 1 | | | | | | | |
| 9 | 3.6 | (12) | 2.0/4.9 | (9) | AA015331 | Cstb | cystatin B | + | + | | | | | |
| 10 | 4.4 | (7) | 2.1 | (14) | X80478 | Aebp1 | AE binding | + | | | | | | |
| | | | | | | | protein 1 | | | | | | | |
| 11 | 6.1 | (4) | 2.0 | (18) | aa238081 | C1r | complement | + | | | | | | |
| | | | | | | | component 1. r | | | | | | | |
| | | | | | | | subcomponent | | | | | | | |
| | | | | | | | tumor necrosis | | | | | | | |
| | | | | | | | factor receptor | | | | | | | |
| | | | | | | | superfamily. | | | | | | | |
| | | | | | | | member | | | | | | | |
| 12 | 2.4 | (17) | 3.8 | (6) | m59378 | Tnfrsf1b | 1b | + | | | | | | |
| 13 | 2.4 | (16) | 4.3/2.9/3.5 | (7) | aa140446 | Tm7sf1 | transmembrane 7 | + | | | | | | |
| | | | | | | | superfamily | | | | | | | |
| | | | | | | | member 1 | | | | | | | |
| 14 | 7.3 | (3) | 1.9 | (20) | v00755 | Timp1 | tissue inhibitor | + | + | | | | | |
| | | | | | | | of metallo- | | | | | | | |
| | | | | | | | proteinase 1 | | | | | | | |
| | | | | | | | proteasome | | | | | | | |
| | | | | | | | (prosome. | | | | | | | |
| | | | | | | | macropain) | | | | | | | |
| | | | | | | | subunit. beta | | | | | | | |
| 15 | 3.6 | (11) | 2.5/2.0 | (13) | I11145 | Psmb8; | type. 8 | + | | + | | | | |
| | | | | | | Lmp7 | | | | | | | | |
| 16 | 2.4 | (18) | 3.5 | (8) | af006467 | Pitpnm | phosphatidyl- | + | | + | | | | |
| | | | | | | | inositol | | | | | | | |
| | | | | | | | membrane- | | | | | | | |
| | | | | | | | associated | | | | | | | |
| 17 | 3.6 | (13) | 2.0 | (17) | m89956 | Lsp1 | lymphocyte | + | | | | | | |
| | | | | | | | specific 1 | | | | | | | |
| 18 | 2.4 | (19) | 2.4 | (12) | u19482 | Ccl9; | chemokine | + | + | | | | | |
| | | | | | | MIP-1y | (C-C motif) | | | | | | | |
| | | | | | | | ligand 9 | | | | | | | |
| 19 | 2.6 | (14) | 2.0/1.8 | (19) | M86736 | Gm | granulin | + | + | | | | | |
| 20 | 2.4 | (20) | 2.0 | (16) | aa518531 | Mylc2b | Myosin light | + | | | | | | |
| | | | | | | | chain. | | | | | | | |
| | | | | | | | regulatory B | | | | | | | |
| 21 | 2.4 | (21) | 1.8 | (21) | AA154294 | Dusp1 | dual specificity | + | | | | | | |
| | | | | | | | phosphatase 1 | | | | | | | |
| 22 | 2.4 | (22) | 1.8 | (22) | X58861 | C1qa | complement | + | | | | | | |
| | | | | | | | component 1. q | | | | | | | |
| | | | | | | | subcomponent. | | | | | | | |
| | | | | | | | alpha | | | | | | | |
| 1 | 4.6 | (1) | 0.3/0.2 | (4) | W10526 | Cacng1 | calcium channel. | + | | | | | | WJ |
| | | | | | | | voltage- | | | | | | | down |
| | | | | | | | dependent. | | | | | | | |
| | | | | | | | gamma | | | | | | | |
| | | | | | | | subunit 1 | | | | | | | |
| 2 | 3.5 | (3) | 0.3 | (2) | w08218 | Tnnt1 | troponin T1. | + | | | | | | |
| | | | | | | | skeletal. slow | | | | | | | |
| 3 | 3.2 | (6) | 0.3 | (3) | J04992 | Tnni2 | troponin I. | + | | | | | | |
| | | | | | | | skeletal. fast 2 | | | | | | | |
| 4 | 3.5 | (2) | 0.6 | (8) | X62940 | Tgfb1i4 | transforming | + | | | | | | |
| | | | | | | | growth factor | | | | | | | |
| | | | | | | | beta 1 induced | | | | | | | |
| | | | | | | | transcript 4 | | | | | | | |
| 5 | 3.2 | (5) | 0.4 | (5) | x14194 | Nid1 | nidogen 1 | + | | | | | | |
| 6 | 2.8 | (9) | 0.2 | (1) | X98014 | Siat8e | alpha-2.8-sialyl- | + | | + | | | | |
| | | | | | | | transferase. | | | | | | | |
| 7 | 3.2 | (4) | 0.7 | (9) | X04405 | Mb | myoglobin | + | + | | + | | +* | |
| 8 | 2.8 | (7) | 0.4/0.4 | (7) | AB001607 | Ptgis | prostaglandin I2 | + | | | + | | | |
| | | | | | | | (prostacyclin) | | | | | | | |
| | | | | | | | synthase | | | | | | | |
| 9 | 2.8 | (8) | 0.4 | (6) | AA152678 | Bsg | basigin | − | + | | + | | +* | |

R refers to the differential expression likehood of any gene to its corresponding library at 99% significance level. FC refers to fold change over control, as identified by the microarray hybridizations of the Tg197 samples at 90% significance level. Multiple entries indicate multiple microarray probesets directed against the same gene. Numbers in parentheses indicate the corresponding ranks. The column Rank lists the average rank. The differential expression was validated with a number of different methods as described in the text. TNF^{ΔARE+/-}; deregulated expression in the knock in mouse model of RA; literature: genes associated with Arthritis upon automated text mining of literature; QTL: genes co-localized with CIA QTLs; (S)Q-RT-PCR: genes confirmed with (Semi) Quantitative RT-PCR in mouse (m) or human (h) samples; +/− indicate agreement/disagreement respectively.
*denotes statistical significance

TABLE 3

| | | | | m SQ- | | |
|---|---|---|---|---|---|---|
| | | | | RT- | | |
| Gene | Genbank | Description | Expression | PCR | WJ | SF |
| Hp_SF | M96827 | haptoglobin | SF Up | Y | | |
| Timp3 | z30970 | tissue inhibitor of metalloproteinase 3 | SF Up | N | Down | |
| Saa3 | x03479 | serum amyloid A 3 | SF Up | N/A | | |
| Rab14 | W41762 | RAB14, (GTPase) member RAS oncogene | SF Up | Y | | |
| | | family | | | | |
| Mmp3 | x66402 | matrix metalloproteinase 3 | SF Up | Y | Up | |
| Mmp13 | X66473 | matrix metalloproteinase 13 | SF Up | Y | | |
| AI451450 | aa674986 | expressed sequence AI451450 | SF Up | N/A | | |
| C3 | k02782 | complement component 3 | SF Up | Y | Up | |
| cdc42 | AA096992 | cell division cycle 42 homolog | SF Down | Y | | |
| hom | | | | | | |
| Gapdh | M32599 | similar to -glyceraldehyde-3-phosphate | SF Down | N/A | | |
| hom | | dehydrogenase | | | | |
| Mglap | D00613 | matrix gamma-carboxyglutamate (gla) | SF Down | Y | | |
| | | protein | | | | |
| Tuba1 | M13445 | tubulin. alpha 1 | SF Down | Y | | |
| Gsn | J04953 | gelsolin | SF Down | Y | | |
| Eef1a1 | W44091 | elongation factor 1-alpha | SF Down | Y | Down | |
| Aqp1 | L02914 | aquaporin 1 | SF Down | Y | | |
| Psmb8; | I11145 | proteasome (prosome. macropain) | WJ Up | Y | | Up |
| Lmp7 | | subunit. beta type. 8 | | | | |
| Lst1 | U72643 | leukocyte specific transcript 1 | WJ Up | Y | | |
| Tm7sf1 | aa140446 | transmembrane 7 superfamily member 1 | WJ Up | N/A | | |
| Tnfrsf1b | m59378 | tumor necrosis factor receptor superfamily. | WJ Up | Y | | Up |
| | | member 1b | | | | |
| C1qa | X58861 | complement component 1. q | WJ Up | Y | | Down |
| | | subcomponent, alpha | | | | |
| Grn | M86736 | granulin | WJ Up | Y | | |
| Hp_WJ | M96827 | haptoglobin | WJ Up | Y | | |
| Timp1 | v00755 | tissue inhibitor of metalloproteinase 1 | WJ Up | N | | Up |
| Pstpip1 | AA038079 | proline-serine-threonine phosphatase- | WJ Up | N/A | | |
| | | interacting protein 1 | | | | |
| Pltpnm | af006467 | phosphatidylinositol membrane-associated 1 | WJ Up | Y | | Up |
| Ncf1 | L11455 | neutrophil cytosolic factor 1 | WJ Up | Y | | Up |
| Mylc2b | aa518531 | Myosin light chain, regulatory B | WJ Up | Y | | Down |
| Marco | u18424 | macrophage receptor with collagenous | WJ Up | Y | | |
| | | structure | | | | |
| Lsp1 | m89956 | lymphocyte specific 1 | WJ Up | Y | | Down |
| Itgax | AA170355 | integrin alpha X | WJ Up | N/A | | |
| Dusp1 | AA154294 | dual specificity phosphatase 1 | WJ Up | N | | Up |
| Cstb | AA015331 | cystatin B | WJ Up | N | | Up |
| C1r | aa238081 | complement component 1, r | WJ Up | Y | | |
| | | subcomponent | | | | |
| Ccl9: | u19482 | chemokine (C-C motif) ligand 9 | WJ Up | Y | | UP |
| MIP-1γ | | | | | | |
| Cd14 | x13333 | CD14 antigen | WJ Up | Y | | |
| Ctss | AA089333 | cathepsin S | WJ Up | N/A | | |
| Aebp1 | X80478 | AE binding protein 1 | WJ Up | Y | | Up |
| Nid1 | x14194 | nidogen 1 | WJ Down | Y | | |
| Slat8e | X98014 | alpha-2.8-sialyltransferase. | WJ Down | Y | | |
| Tnnt1 | w08218 | troponin T1. skeletal. slow | WJ Down | Y | | |
| Tnni2 | J04992 | troponin I. skeletal. fast 2 | WJ Down | Y | | |
| Tgfb1i4 | X62940 | transforming growth factor beta 1 induced | WJ Down | Y | | SF |
| | | transcript 4 | | | | Down |
| Ptgis | AB001607 | prostaglandin 12 (prostacyclin) synthase | WJ Down | Y | | |
| Mb | X04405 | myoglobin | WJ Down | Y | | |
| Cacng1 | W10526 | calcium channel, voltage-dependent, | WJ Down | Y | | |
| | | gamma subunit 1 | | | | |
| Bsg | AA152678 | basigin | WJ Down | Y | | |

TABLE 4
Key regulators of actin-cytoskeleton dynamics and organization.

| Gene | Description | Reference |
|---|---|---|
| Arp2/3 complex | Crucial for initiation of actin assembly, nucleation | Svitkina et al, 1999 |
| | of new filaments and actin polymerisation | |
| WASP/WAVE/WIP | Activates the Arp2/3 complex | Rohatzi et al, 1999; |
| | | Gallego et al, 2005 |
| SCAR | Promotes actin nucleation via the Arp2/3 complex | Machesky et al, 1999 |
| ERM (Ezrin, | A predominant role in intracellular positioning of | Gautreau et al, 2002 |
| Radixin, Moesin) | actin polymerisation and signalling | |
| Zyxin | A predominant role in intracellular positioning of | Hoffman et al, 2006 |
| | actin polymerisation and signalling | |
| VASP | Promotes F-actin formation and regulates actin | Galler et al, 2006 |
| | polymerisation | |
| Cortactin | Stabilises actin filaments, promotes actin | Weaver et al, 2001 |
| | nucleation | |
| Profilin | Sequesters actin monomers, releases G-actin- | Schluter et al, 1997 |
| | ATP, promotes actin polymerisation | |
| Thymosin | Co-operates with profilin to control the pool of un- | Carlier et al, 1993 |
| | polymerised actin | |
| PI(4,5)P₂ | Activates actin polymerisation through the N- | Rohatzi et al, 2001 |
| | WASP-Arp2/3 pathway | |
| Coronin 1 | Inhibits Arp2/3 complex at the leading edge and | Foger et al, 2006 |
| | blocks F-actin accumulation | |
| Tropomyosin | Inhibits F-actin formation through the Arp2/3 | Nyakern-Meazza, 2002 |
| | complex | |
| Caldesmon | Inhibits F-actin formation through the Arp2/3 | Kordowska et al, 2006 |
| | complex | |
| CapZ | (+)end capping protein, inhibits actin | Cooper and Schafer, 2000 |
| | polymerization | |
| Tropomodulin | (−)end capping protein, inhibits actin | Fujisawa et al, 2001 |
| | polymerization | |
| ADF/Cofilin | Binds to G-actin-ADP, inhibits actin re- | Hotulainer et al, 2005 |
| | polymerization | |
| AIP-1 | Interacts with cofilin and caps the (+)end of a | Rodal et al, 1999 |
| | cofilin-severed filament | |
| Twinfilin | Inhibits the assembly of actin monomers | Moseley et al, 2006 |
| Vinculin | A focal adhesion protein that guides the Arp2/3 | DeMali et al, 2002 |
| | complex to the cell-substratum | |
| RhoA, Rac and | RhoA GTPases promote actin polymerisation by | Lamarche et al, 1996 |
| Cdc42 | activating nucleation by Arp2/3 complex and by | |
| | inhibiting depolymerisation by ADF/cofilins | |
| ROCK | A Rho-associated kinase that activates LIM | Pawlak et al, 2002 |
| | kinase, which dephosphorylates cofilin, induces | |
| | actin formation | |
| FilGAP | A Rho- and ROCK-regulated GAP for Rac binds | Ohta et al, 2006 |
| | filamin A to control actin remodelling | |
| SRF | A transcription factor regulating the expression of | Schratt et al, 2002 |
| | several actin-cytoskeleton genes inducing actin | |
| | polymerization | |
| Autotaxin | Hyrdolyses LPC to LPA, a crucial lipid mediator | Song et al, 2005 |
| | functioning in cell proliferation, migration and | |
| | survival | |
| SHIP2 | Regulates localised changes in PIP3, instructs | von Wichert et al, 2003 |
| | actin remodelling, membrane ruffling and | |
| | promotes cell adhesion | |

TABLE 5
mouse models of Rheumatoid Arthritis

| | | |
|---|---|---|
| Mouse model | Description | Reference |

Induced

| | | |
|---|---|---|
| Collagen-induced arthritis | T and B cell immunity to autologous | Trentham et al, 1977 |
| (CIA) | type II collagen | |
| Pristane-induced arthritis | Systemic (intra-peritoneal) injection of | Elson et al, 1992 |
| | pristine | |
| Proteoglycan-induced arthritis | Systemic injection of BALB/c mice with | Glant et al, 2003 |
| | proteoglycan aggrecan emulsified in | |
| | DDA adjuvant | |
| Streptococcal cell-wall induced | Intra-articular injection of cell walls from | Van den Broek et al, 1988 |
| arthritis | Streptococcus pyogenes | |
| Immune-complex induced | Intravenous injection with heat- | Van Lent et al, 2003 |
| arthritis | inactivated polyclonal rabbit anti- | |
| | lysozyme serum, followed by injection | |
| | with poly-L-lysine-coupled lysozyme in | |
| | the joint | |
| Zymosan-induced arthritis | Intra-articular injection of zymosan A | Schalkwijk et al, 1985 |
| | and development of irritant-induced | |
| | arthritis | |

Genetic

| | | |
|---|---|---|
| Ncf1 | Mutation in Ncfl leading to truncated | Hultqvist et a, 2004 |
| | protein and lower levels of reactive | |
| | oxygen species in NADPH oxidase- | |
| | expressing cells | |
| Zap70 | Altered thymic T-cell selection due to a | Sakaguchi et al, 2003 |
| | mutation of the ZAP-70 gene causes | |
| | autoimmune arthritis in mice | |
| Fas-related arthropathy | A spontaneous rheumatoid arthritis-like | Hang et al, 1982 |
| | disease in MRL/I mice | |
| K/BxN | KRN T-cell receptor transgenic mouse | Kouskoff et al, 1996 |
| | on the C57BL/6xNOD background | |
| HTLV-1 | Induction of inflammatory arthropathy | Iwakura et al, 1991 |
| | resembling rheumatoid arthritis in mice | |
| | transgenic for HTLV-I | |
| hIL-1a | Transgenic mice expressing hIL-1 alpha | Niki et al, 2001 |
| | mRNA in various organs, had high | |
| | serum levels of hIL-1 alpha, and | |
| | developed a severe polyarthritic | |
| | phenotype at 4 weeks of age. | |
| IL-1ra | IL-1ra deficient mice, on a BALB/cA | Horai et al, 2000 |
| | background, develop chronic | |
| | inflammatory polyarthropathy | |
| IL-6 | A point mutation of Tyr-759 in | Atsumi et al, 2002 |
| | interleukin 6 family cytokine receptor | |
| | subunit gp130 causes autoimmune | |
| | arthritis | |
| hTNF-α | Transgenic mice expressing high levels | Keffer et al, 1991 |
| | of both soluble and membrane-bound | |
| | hTNF-α develop chronic inflammatory | |
| | polyarthritis | |
| TNF^{ΔARE} | Deletion of a TNF AUUUA motif results | Kontoyiannis et al, 1999 |
| | in TNF overexpression and chronic | |
| | inflammatory arthritis | |
| TTP | Tristetraprolin-deficient mice develop | Taylor et al, 1996 |
| | inflammatory arthritis due to de- | |
| | stabilisation of TNF-α mRNA | |
| Synoviolin/Hrd1 | Over-expression of human | Amano et al, 2006 |
| | Synoviolin/Hrd1leads to spontaneous | |
| | arthropathy | |

TABLE 6
New mouse models of Rheumatoid arthritis from crosses with Tg197 and involvement of
cytoskeletal organization in the pathogenesis of RA (KO, knockout; GT, Gene Trap).

| Mouse model | Source | Crossing | Phenotype |
|---|---|---|---|
| VASP KO | Ulrich Walter, Germany | Tg197 | Amelioration |
| COFILIN 1 KO | Walter Witke, Italy | Tg197 | Exacerbation |
| LST-1 GT | SIGTR, Sanger Institute, UK | Tg197 | Amelioration |
| Rac1 conditional KO | Cord Brakebusch, Germany | ColVI-Cre/Tg197 | Amelioration/SF specific |
| RhoA conditional KO | CordBrakebusch, Germany | ColVI-Cre/Tg197 | Amelioration/SF specific |
| Cdc42 conditional KO | Cord Brakebusch, Germany | ColVI-Cre/Tg197 | Amelioration/SF specific |
| SRF conditional KO | Alfred Nordheim, Germany | ColVI-Cre/Tg197 | Amelioration/SF specific |
| ATX conditional KO | Vasilis Aidinis, Greece | ColVI-Cre/Tg197 | Amelioration/SF specific |

TABLE A

| | | | | | | | | | | m SQ- | m Q- | h Q- | |
|---|---|---|---|---|---|---|---|---|---|---|---|---|---|
| | | | | | | | | Liter- | | RT- | RT- | RT- | |
| R* | | FC* | | Genbank | Name | Description | TNF^{ΔARE+/-} | ature | QTL | PCR | PCR | PCR | |
| 2.9 | (2) | 2.7 | (4) | aa674986 | AI451450 | expressed sequence AI451450 | + | | | | | | SF |
| | | | | | | | | | | | | | up |
| 3.3 | (4) | 0.3 | (2) | AA096992 | cdc42 hom | cell division cycle 42 | + | | | + | | − | SF |
| | | | | | | homolog | | | | | | | down |
| 2.6 | (5) | 0.5 | (4) | D00613 | Mglap | matrix gamma-carboxy- | + | | | + | | | |
| | | | | | | glutamate (gla) protein | | | | | | | |
| 4.7 | (3) | 0.8 | (7) | M32599 | Gapdh hom | similar to -glyceraldehyde- | − | | | | | | |
| | | | | | | 3-phosphate dehydrogenase | | | | | | | |
| 3.6 | (9) | 17.7 | (1) | AA170355 | Itgax | integrin alpha X | + | | | | | | WJ |
| 2.4 | (15) | 7.3 | (3) | U18424 | Marco | macrophage receptor with | − | | | + | | + | up |
| | | | | | | collagenous structure | | | | | | | |
| 3.6 | (10) | 3.4 | (10) | U72643 | Lst1 | leukocyte specific | + | | | | | | |
| | | | | | | transcript 1 | | | | | | | |
| 4.4 | (7) | 2.1 | (14) | X80478 | Aebp1 | AE binding protein 1 | + | | | | | | |
| 6.1 | (4) | 2.0 | (18) | aa238081 | C1r | complement component 1. r | + | | | | | | |
| | | | | | | subcomponent | | | | | | | |
| 2.4 | (17) | 3.8 | (6) | m59378 | Tnfrsf1b | tumor necrosis factor | + | | | | | | |
| | | | | | | receptor superfamily. | | | | | | | |
| | | | | | | member 1b | | | | | | | |
| 2.4 | (16) | 4.3/2.9/3.5 | (7) | aa140446 | Tm7sf1 | transmembrane 7 super- | + | | | | | | |
| | | | | | | family member 1 | | | | | | | |
| 3.6 | (13) | 2.0 | (17) | m89956 | Lsp1 | lymphocyte specific 1 | + | | | | | | |
| 2.4 | (20) | 2.0 | (16) | aa518531 | Mylc2b | Myosin light chain, | + | | | | | | |
| | | | | | | regulatory B | | | | | | | |
| 2.4 | (22) | 1.8 | (22) | X58861 | C1qa | complement component 1. | + | | | | | | |
| | | | | | | q subcomponent alpha | | | | | | | |
| 4.6 | (1) | 0.3/0.2 | (4) | W10526 | Cacng1 | calcium channel. voltage- | + | | | | | | WJ |
| | | | | | | dependent, gamma subunit 1 | | | | | | | down |
| 3.5 | (3) | 0.3 | (2) | w08218 | Tnnt1 | troponin T1 skeletal. slow | + | | | | | | |
| 3.2 | (6) | 0.3 | (3) | J04992 | Tnni2 | troponln I. skeletal. | + | | | | | | |
| | | | | | | fast 2 | | | | | | | |
| 3.5 | (2) | 0.6 | (8) | X62940 | Tgfb1i4 | transforming growth factor | + | | | | | | |
| | | | | | | beta 1 induced transcript 4 | | | | | | | |
| 3.2 | (5) | 0.4 | (5) | x14194 | Nid1 | nidogen 1 | + | | | | | | |
| 2.8 | (7) | 0.4/0.4 | (7) | AB001607 | Ptgis | prostaglandin I2 | + | | | + | | | |
| | | | | | | (prostacyclin) synthase | | | | | | | |

TABLE B

| | | | | | | | | | | m SQ- | m Q- | h Q- | |
|---|---|---|---|---|---|---|---|---|---|---|---|---|---|
| | | | | | | | | Liter- | | RT- | RT- | RT- | |
| R* | | FC* | | Genbank | Name | Description | TNF^{ΔARE+/-} | ature | QTL | PCR | PCR | PCR | |
| 3.7 | (6) | 1.3 | (8) | W41762 | Rab14 | RAB14. (GTPase) member | + | | + | +/− | − | | SF |
| | | | | | | RAS oncogene family | | | | | | | up |
| 2.6 | (6) | 0.7 | (6) | J04953 | Gsn | Gelsolin | − | | + | + | + | +* | SF |
| | | | | | | | | | | | | | down |
| 3.6 | (11) | 2.5/2.0 | (13) | I11145 | Psmb8; | proteasome (prosome. | + | | + | | | | WJ |
| | | | | | Lmp7 | macropain) subunit. | | | | | | | up |
| | | | | | | beta type. 8 | | | | | | | |
| 2.4 | (18) | 3.5 | (8) | af006467 | Pitpnm | phosphatidylinositol | + | | + | | | | |
| | | | | | | membrane-associated | | | | | | | |
| 2.8 | (9) | 0.2 | (1) | X98014 | Siat8e | alpha-2.8- | + | | + | | | | WJ |
| | | | | | | sialyltransferase. | | | | | | | down |

TABLE C

| | | | | | | | | | | m SQ- | m Q- | h Q- | |
|---|---|---|---|---|---|---|---|---|---|---|---|---|---|
| | | | | | | | | Liter- | | RT- | RT- | RT- | |
| R* | | FC* | | Genbank | Name | Description | TNF^{ΔARE+/-} | ature | QTL | PCR | PCR | PCR | |
| 5.2 | (4) | 11.4 | (1) | M96827 | Hp | haptoglobin | + | + | | + | | | SF |
| 2.8 | (1) | 2.6 | (5) | x66402 | Mmp3 | matrix metalloproteinase 3 | − | + | | | | | up |
| 4.4 | (5) | 3.2 | (3) | k02782 | C3 | complement component 3 | + | + | | | | | |
| 3.3 | (8) | 3.6 | (2) | X66473 | Mmp13 | matrix metalloproteinase 13 | + | + | | | | | |
| 5.9 | (3) | 1.4 | (7) | z30970 | Timp3 | tissue inhibitor of | + | + | | | | | |
| | | | | | | metalloproteinase 3 | | | | | | | |
| 3.7 | (7) | 2.6 | (6) | x03479 | Saa3 | serum amyloid A 3 | + | + | | | | | |
| 6.7 | (1) | 0.3 | (1) | W44091 | Eef1a1 | elongation factor 1-alpha | + | + | | | | | SF |
| 6.6 | (2) | 0.7 | (5) | M13445 | Tuba1 | tubulin. alpha 1 | − | + | | +/− | | | down |
| 2.3 | (7) | 0.4 | (3) | L02914 | Aqp1 | aquaporin 1 | + | + | + | + | + | + | |
| 9.7 | (1) | 5.5/2.7 | (5) | AA089333 | Ctss | cathepsin S | + | + | + | | | | WJ |
| 4.8 | (5) | 10.8 | (2) | AA038079 | Pstpip1 | proline-serine-threonine | + | + | | | | | up |
| | | | | | | phosphatase-interacting | | | | | | | |
| | | | | | | protein 1 | | | | | | | |
| 4.8 | (6) | 4.2 | (4) | x13333 | Cd14 | CD14 antigen | + | + | | + | | + | |
| 7.3 | (2) | 2.1 | (15) | M96827 | Hp | haptoglobin | + | + | | + | | | |
| 3.7 | (8) | 2.7 | (11) | L11455 | Ncf1 | neutrophil cytosolic | + | + | + | | | | |
| | | | | | | factor 1 | | | | | | | |
| 3.6 | (12) | 2.0/4.9 | (9) | AA015331 | Cstb | cystatin B | + | + | | | | | |
| 7.3 | (3) | 1.9 | (20) | v00755 | Timp1 | tissue inhibitor of | + | + | | | | | |
| | | | | | | metalloproteinase 1 | | | | | | | |
| 2.4 | (19) | 2.4 | (12) | u19482 | Ccl9; | chemokine (C-C motif) | + | + | | | | | |
| | | | | | MIP-1γ | ligand 9 | | | | | | | |
| 2.6 | (14) | 2.0/1.8 | (19) | M86736 | Grn | granulin | + | + | | | | | |
| 3.2 | (4) | 0.7 | (9) | X04405 | Mb | myoglobin | + | + | | + | | +* | WJ |
| 2.8 | (8) | 0.4 | (6) | AA152678 | Bsg | basigin | − | + | | + | | +* | down |

### REFERENCES

1. Choy E H, Panayi G S (2001) Cytokine pathways and joint inflammation in rheumatoid arthritis. N Engl J Med 344: 907-916.
2. Smolen J S, Steiner G (2003) Therapeutic strategies for rheumatoid arthritis. Nat Rev Drug Discov 2: 473-488.
3. Firestein G S (2003) Evolving concepts of rheumatoid arthritis. Nature 423: 356-361.
4. Lindqvist A K B, Bockermann R, Johanson A C M, Nandakumar K S, Johannensson M, et al. (2002) Mouse models for rheumatoid arthritis. Trends Genet. 18: S7-S13.
5. Keffer J, Probert L, Cazlaris H, Georgopoulos S, Kaslaris E, et al. (1991) Transgenic mice expressing human tumour necrosis factor: a predictive genetic model of arthritis. Embo J 10: 4025-4031.
6. Li P, Schwarz E M (2003) The TNF-alpha transgenic mouse model of inflammatory arthritis. Springer Semin Immunopathol 25: 19-33.
7. Aidinis V, Plows D, Haralambous S, Armaka M, Papadopoulos P, et al. (2003) Functional analysis of an arthritogenic synovial fibroblast. Arthritis Res Ther 5: R140-157.
8. Kontoyiannis D, Pasparakis M, Pizarro T T, Cominelli F, Kollias G (1999) Impaired on/off regulation of TNF biosynthesis in mice lacking TNF AU-rich elements: implications for joint and gut-associated immunopathologies. Immunity 10: 387-398.
9. Brazma A, Hingamp P, Quackenbush J, Sherlock G, Spellman P, et al. (2001) Minimum information about a microarray experiment (MIAME)-toward standards for microarray data. Nat Genet. 29: 365-371.
10. Brazma A, Parkinson H, Sarkans U, Shojatalab M, Vilo J, et al. (2003) ArrayExpress—a public repository for microarray gene expression data at the EBI. Nucleic Acids Res 31: 68-71.
11. Pavelka N, Pelizzola M, Vizzardelli C, Capozzoli M, Splendiani A, et al. (2004) A power law global error model for the identification of differentially expressed genes in microarray data. BMC Bioinformatics 5: 203.
12. Irizarry R A, Warren D, Spencer F, Kim I F, Biswal S, et al. (2005) Multiple-laboratory comparison of microarray platforms. Nat Methods 2: 345-350.
13. Marshall E (2004) Getting the noise out of gene arrays. Science 306: 630-631.
14. Trentham D E, Townes A S, Kang A H (1977) Autoimmunity to type II collagen an experimental model of arthritis. J Exp Med 146: 857-868.
15. Neumann E, Kullmann F, Judex M, Justen H P, Wessinghage D, et al. (2002) Identification of differentially expressed genes in rheumatoid arthritis by a combination of complementary DNA array and RNA arbitrarily primed-polymerase chain reaction. Arthritis Rheum 46: 52-63.
16. van der Pouw Kraan T C, van Gaalen F A, Kasperkovitz P V, Verbeet N L, Smeets T J, et al. (2003) Rheumatoid arthritis is a heterogeneous disease: evidence for differences in the activation of the STAT-1 pathway between rheumatoid tissues. Arthritis Rheum 48: 2132-2145.
17. Ashburner M, Ball C A, Blake J A, Botstein D, Butler H, et al. (2000) Gene ontology: tool for the unification of biology. The Gene Ontology Consortium. Nat Genet. 25: 25-29.
18. Witke W, Sharpe A H, Hartwig J H, Azuma T, Stossel T P, et al. (1995) Hemostatic, inflammatory, and fibroblast responses are blunted in mice lacking gelsolin. Cell 81: 41-51.
19. Olofsson P, Holmberg J, Tordsson J, Lu S, Akerstrom B, et al. (2003) Positional identification of Ncf1 as a gene that regulates arthritis severity in rats. Nat Genet. 33: 25-32.
20. Saadoun S, Papadopoulos M C, Hara-Chikuma M, Verkman A S (2005) Impairment of angiogenesis and cell migration by targeted aquaporin-1 gene disruption. Nature 434: 786-792.
21. Gabbiani G (2003) The myofibroblast in wound healing and fibrocontractive diseases. J Pathol 200: 500-503.
22. Tomasek J J, Gabbiani G, Hinz B, Chaponnier C, Brown R A (2002) Myofibroblasts and mechano-regulation of connective tissue remodelling. Nat Rev Mol Cell Biol 3: 349-363.
23. Mattey D L, Dawes P T, Nixon N B, Slater H (1997) Transforming growth factor beta 1 and interleukin 4 induced alpha smooth muscle actin expression and myofibroblast-like differentiation in human synovial fibroblasts in vitro: modulation by basic fibroblast growth factor. Ann Rheum Dis 56: 426-431.
24. Gumbiner B M (1996) Cell adhesion: the molecular basis of tissue architecture and morphogenesis. Cell 84: 345-357.
25. Kasperkovitz P V, Timmer T C, Smeets T J, Verbeet N L, Tak P P, et al. (2005) Fibroblast-like synoviocytes derived from patients with rheumatoid arthritis show the imprint of synovial tissue heterogeneity: evidence of a link between an increased myofibroblast-like phenotype and high-inflammation synovitis. Arthritis Rheum 52: 430-441.
26. Shrivastav M, Mittal B, Aggarwal A, Misra R (2002) Autoantibodies against cytoskeletal proteins in rheumatoid arthritis. Clin Rheumatol 21: 505-510.
27. von Muhlen C A, Tan E M (1995) Autoantibodies in the diagnosis of systemic rheumatic diseases. Semin Arthritis Rheum 24: 323-358.
28. van Boekel M A, Vossenaar E R, van den Hoogen F H, van Venrooij W J (2002) Autoantibody systems in rheumatoid arthritis: specificity, sensitivity and diagnostic value. Arthritis Res 4: 87-93.
29. Presland R B, Kuechle M K, Lewis S P, Fleckman P, Dale B A (2001) Regulated expression of human ffilaggrin in keratinocytes results in cytoskeletal disruption, loss of cell-cell adhesion, and cell cycle arrest. Exp Cell Res 270: 199-213.
30. Vossenaar E R, van Venrooij W J (2004) Citrullinated proteins: sparks that may ignite the fire in rheumatoid arthritis. Arthritis Res Ther 6: 107-111.
31. Pawlak G, Helfman D M (2001) Cytoskeletal changes in cell transformation and tumorigenesis. Curr Opin Genet Dev 11: 41-47.
32. Firestein G S (1996) Invasive fibroblast-like synoviocytes in rheumatoid arthritis. Passive responders or transformed aggressors? Arthritis Rheum 39: 1781-1790.
33. dos Remedios C G, Chhabra D, Kekic M, Dedova I V, Tsubakihara M, et al. (2003) Actin binding proteins: regulation of cytoskeletal microfilaments. Physiol Rev 83: 433-473.
34. McGough A M, Staiger C J, Min J K, Simonetti K D (2003) The gelsolin family of actin regulatory proteins: modular structures, versatile functions. FEBS Lett 552: 75-81.
35. Vandekerckhove J, Bauw G, Vancompernolle K, Honore B, Celis J (1990) Comparative two-dimensional gel analysis and microsequencing identifies gelsolin as one of the most prominent downregulated markers of transformed human fibroblast and epithelial cells. J Cell Biol 111: 95-102.
36. Mullauer L, Fujita H, Ishizaki A, Kuzumaki N (1993) Tumor-suppressive function of mutated gelsolin in ras-transformed cells. Oncogene 8: 2531-2536.
37. Asch H L, Head K, Dong Y, Natoli F, Winston J S, et al. (1996) Widespread loss of gelsolin in breast cancers of humans, mice, and rats. Cancer Res 56: 4841-4845.
38. Tanaka M, Mullauer L, Ogiso Y, Fujita H, Moriya S, et al. (1995) Gelsolin: a candidate for suppressor of human bladder cancer. Cancer Res 55: 3228-3232.
39. Dosaka-Akita H, Hommura F, Fujita H, Kinoshita I, Nishi M, et al. (1998) Frequent loss of gelsolin expression in non-small cell lung cancers of heavy smokers. Cancer Res 58: 322-327.
40. Aidinis V, Plows, D., Haralambous, S., Armaka, M., Papadopoulos, P., Kanaki, M-Z., Koczan, D., Thiesen, H. J. and Kollias, G. (2002) Differential gene expression analysis of an arthritic synovial fibroblast reveals its functional properties. submitted.
41. Chomczynski P, Sacchi N (1987) Single-step method of RNA isolation by acid guanidinium thiocyanate-phenol-chloroform extraction. Anal Biochem 162: 156-159.
42. Carninci P, Hayashizaki Y (1999) High-efficiency full-length cDNA cloning. Methods Enzymol 303: 19-44.
43. Shibata Y, Carninci P, Watahiki A, Shiraki T, Konno H, et al. (2001) Cloning full-length, cap-trapper-selected cDNAs by using the single-strand linker ligation method. Biotechniques 30: 1250-1254.
44. Carninci P, Shibata Y, Hayatsu N, Itoh M, Shiraki T, et al. (2001) Balanced-size and long-size cloning of full-length, cap-trapped cDNAs into vectors of the novel lambda-FLC family allows enhanced gene discovery rate and functional analysis. Genomics 77: 79-90.
45. Hirozane-Kishikawa T, Shiraki T, Waki K, Nakamura M, Arakawa T, et al. (2003) Subtraction of cap-trapped full-length cDNA libraries to select rare transcripts. Biotechniques 35: 510-516, 518.
46. Shibata K, Itoh M, Aizawa K, Nagaoka S, Sasaki N, et al. (2000) RIKEN integrated sequence analysis (RISA) system—384-format sequencing pipeline with 384 multicapillary sequencer. Genome Res 10: 1757-1771.
47. Carninci P, Waki K, Shiraki T, Konno H, Shibata K, et al. (2003) Targeting a complex transcriptome: the construction of the mouse full-length cDNA encyclopedia. Genome Res 13: 1273-1289.
48. Konno H, Fukunishi Y, Shibata K, Itoh M, Carninci P, et al. (2001) Computer-based methods for the mouse full-length cDNA encyclopedia: real-time sequence clustering for construction of a nonredundant cDNA library. Genome Res 11: 281-289.
49. Miller R T, Christoffels A G, Gopalakrishnan C, Burke J, Ptitsyn A A, et al. (1999) A comprehensive approach to clustering of expressed human gene sequence: the sequence tag alignment and consensus knowledge base. Genome Res 9: 1143-1155.
50. Burke J, Davison D, Hide W (1999) d2_cluster: a validated method for clustering EST and full-length cDNAsequences. Genome Res 9: 1135-1142.
51. Altschul S F, Gish W, Miller W, Myers E W, Lipman D J (1990) Basic local alignment search tool. J Mol Biol 215: 403-410.
52. Altschul S F, Madden T L, Schaffer A A, Zhang J, Zhang Z, et al. (1997) Gapped BLAST and PSI-BLAST: a new generation of protein database search programs. Nucleic Acids Res 25: 3389-3402.
53. Okazaki Y, Furuno M, Kasukawa T, Adachi J, Bono H, et al. (2002) Analysis of the mouse transcriptome based on functional annotation of 60,770 full-length cDNAs. Nature 420: 563-573.
54. Kawai J, Shinagawa A, Shibata K, Yoshino M, Itoh M, et al. (2001) Functional annotation of a full-length mouse cDNA collection. Nature 409: 685-690.
55. Stekel D J, Git Y, Falciani F (2000) The comparison of gene expression from multiple cDNA libraries. Genome Res 10: 2055-2061.
56. Rose C, Smith M D (2002) Mathematical Statistics with Mathematica; Verlag S, editor: Springer Verlag.
57. Ihaka R, Gentleman R (1996) A Language for Data Analysis and Graphics. Journal of Computational and Graphical Statistics 5: 299-314.
58. Yuen T, Wurmbach E, Pfeffer R L, Ebersole B J, Sealfon S C (2002) Accuracy and calibration of commercial oligonucleotide and custom cDNA microarrays. Nucleic Acids Res 30: e48.
59. Tsien C L, Libermann T A, Gu X, Kohane I S (2001) On reporting fold differences. Pac Symp Biocomput: 496-507.
60. Tu Y, Stolovitzky G, Klein U (2002) Quantitative noise analysis for gene expression microarray experiments. Proc Natl Acad Sci USA 99: 14031-14036.
61. Mutch D M, Berger A, Mansourian R, Rytz A, Roberts M A (2002) The limit fold change model: a practical approach for selecting differentially expressed genes from microarray data. BMC Bioinformatics 3: 17.
62. Fischer G, Ibrahim S M, Brockmann G A, Pahnke J, Bartocci E, et al. (2003) Expression view: visualization of quantitative trait loci and gene-expression data in Ensembl. Genome Biol 4: R77. Epub 2003 October 2010.
63. Serrano-Fernandez P, Ibrahim, S. M., Godde, R., Epplen, J. & Moller, S. (2004) Intergenomic Consensus in Multifactorial Inheritance Loci: the Case of Multiple Sclerosis. Genes and Immunity 5: 615-620.
64. Tavazoie S, Hughes J D, Campbell M J, Cho R J, Church G M (1999) Systematic determination of genetic network architecture. Nat Genet. 22: 281-285.
65. Mould A W, Tonks I D, Cahill M M, Pettit A R, Thomas R, et al. (2003) Vegfb gene knockout mice display reduced pathology and synovial angiogenesis in both antigen-induced and collagen-induced models of arthritis. Arthritis Rheum 48: 2660-2669.

1. A method for determining whether a compound, a (poly)peptide or a nucleic acid molecule or functional equivalent thereof for altering cytoskeleton or its organization in a cell, affects a phenotype of synovial fibroblast cells of an individual or non-human animal suffering from inflammatory arthritis or at risk of suffering thereof, comprising culturing synovial fibroblast cells in vitro from a sample obtained from an affected joint or a joint at risk of being affected from said individual or non-human animal, in the presence of said compound and/or providing said synovial fibroblast cells with said (poly)peptide or nucleic acid or said functional equivalent thereof and determining whether a phenotype of said cultured synovial fibroblast cells is altered.

2. A method for determining whether a compound, a (poly)peptide or a nucleic acid molecule or functional equivalent thereof affects cytoskeleton or its organization of synovial fibroblast cells of an individual or non-human animal suffering from inflammatory arthritis or at risk of suffering thereof, comprising culturing synovial fibroblast cells in vitro from a sample obtained from an affected joint or a joint at risk of being affected from said individual or non-human animal, in the presence of said compound and/or providing said synovial fibroblast cells with said nucleic acid or said functional equivalent thereof and determining whether cytoskeleton or its organization of said cultured synovial fibroblast cells is altered.

3. A method according to claim 1, wherein said synovial fibroblast cells are obtained from a joint of a mouse comprising the genetic alteration of the Tg197 mouse, a non-human animal according to any one of claims 44-56 and/or a genetic model as depicted in table 5.

4. A method according to claim 1, wherein said compound is effective in cancer therapy.

5. A method according to claim 1, wherein said compound is one of the following: Cytochalasin D, Phalloidin, Jasplakinolide, Chondramides, Dolostatin 11, Latrunculin A, Swinholide A, Misakinolide A, Mycalolide B, Shinxolide C, Scytophycins, Goniodomin A, Pseudopterolide, Lysophosphatidylcholine, Lysophosphatidyl acid.

6. A method according to claim 1, wherein said nucleic acid comprises an antisense-sequence, preferably an RNAi, an siRNA, or an shRNA.

7. A method according to claim 1, wherein said nucleic acid or functional equivalent thereof comprises a morpholino, locked nucleic acid (LNA) or a peptide nucleic acid (PNA).

8. A method according to claim 6 or claim 7, wherein said antisense-sequence is an antisense sequence of a gene listed in table A, B, C, or table 4, wherein said gene is identified in either of said tables by expression SF Up or WJ Up or said gene is VASP.

9. A method according to claim 1, wherein said phenotype comprises adhesion, migration, proliferation, wound-healing and/or apoptosis of said synovial fibroblast cells.

10. A method according to claim 1, wherein said phenotype comprises stress fiber formation.

11. A method according to claim 1, in a high-throughput setting.

12. A method according to claim 1, wherein said nucleic acid codes for a protein produced by a gene of table A, B, C, 3 or 4, wherein said gene is identified in either of said tables by SF down or WJ down or said gene is gelsolin or ADF/Cofilin.

13. Use of a gene delivery vehicle comprising the coding region of a gene of table A, B or C identified by SF down or WJ down, a gene of table 4, Gelsolin or ADF/Cofilin, for delivering said gene to cells of a joint.

14. Use of a gene delivery vehicle according to claim 13, for delivering said gene to a synovial fibroblast cell, or a cell of a whole joint.

15. Use of a gene delivery vehicle comprising the coding region of a gene of table A, B or C identified by SF down or WJ down, Gelsolin or Cofilin, for the preparation of a composition for delivering said gene to cells of a synovial fibroblast cell (SF UP) and/or a joint (WJ UP).

16. Use according to claim 15, for the treatment of inflammatory arthritis.

17. Use according to claim 67, wherein said gene is Gelsolin, ADF/Cofilin.

18. Use of a gene delivery vehicle comprising a nucleic acid of a functional equivalent thereof comprising an antisense-sequence for a gene of table A, B or C identified by SF UP or WJ UP or VASP, or comprising an expression cassette therefore, for delivering said nucleic acid or functional equivalent thereof to a synovial fibroblast cell (SF UP) or to cells of a joint (WJ LTP).

19. Use according to claim 18, wherein said antisense-sequence comprises an shRNA, RNAi and/or siRNA.

20. Use according to claim 13, wherein said gene delivery vehicle comprises a liposome, or a viral vector.

21. Use according to claim 20, wherein said viral vector is an adenovirus, an adeno-associated virus vector or a lentivirus vector.

22. Use according to claim 19, wherein said gene delivery vehicle is administered systemically.

23. Use according to claim 19, wherein said gene delivery vehicle is administered intrarticular.

24. Use according to claim 13, wherein said expression cassette is iven by a pol II promoter or a pol III promoter.

25. A method for determining whether a sample is derived from an individual suffering from inflammatory arthritis or from an individual at risk of suffering therefrom, said method comprising measuring in said sample the level of expression of at least one gene product of a gene of table A, table B, or table 4 and comparing said expression level with a reference value.

26. A method according to claim 25, wherein said gene product comprises RNA.

27. A method according to claim 25, wherein said sample is derived from a joint of said individual.

28. A method according to claim 25, wherein said sample comprises cells of synovial membrane or synovial fluid.

29. A method according to claim 25, wherein said measuring is in cells of said sample.

30. A method according to claim 25, wherein at least two gene products are measured.

31. A method according to claim 25, wherein at least one gene product is of a gene of table A, and/or table 4.

32. A method according to claim 25, wherein at least one gene product is of a gene of table A, table B, and/or table 4 and at least one other gene product is of a gene of table C.

33. A method according to claim 25, wherein at least one gene product is of a gene of table A, or table 4 and at least one other gene product is of a gene of table B or C.

34. A method for determining whether a sample is derived from an individual suffering from inflammatory arthritis or from an individual at risk of suffering thereof, said method comprising measuring in cells from said sample the level of expression of at least one gene product of a gene involved in cytoskeleton arrangement and comparing said expression level with a reference value.

35. A method according to claim 34, wherein said gene is involved in cytoskeleton arrangement in a synovial fibroblast.

36. A method according to claim 34, wherein said gene is involved in extracellular or intracellular signaling to the cytoskeleton.

37. A method according to claim 34, wherein said gene is involved in binding and/or rearrangement of actin cytoskeleton.

38. A method according to claim 34, wherein said gene is any one of the following: tubulin alpha 1, RAB14, Gelsolin, phosphatidylinositol membrane-associated, matrix rnetalloproteinase 3, matrix metalloproteinase 13, tissue inhibitor of matrix metalloproteinase 13, cathepsin S, matrix gamma-carboxylglutamate, integrin alpha X, myosin light chain, troponin T1 skeletal slow, troponin I skeletal fast 2, VASP, ADF/Cofilin and/or is a gene of table 4.

39. A method according to claim 25, wherein said inflammatory arthritis is rheumatoid arthritis.

40. Use of a compound for altering cytoskeleton or its organization in a cell for the manufacture of a medicament for alleviating at least one symptom of inflammatory arthritis in an individual.

41. Use of a compound for altering cytoskeleton or its organization in a cell for reducing stress fibers in a synovial cell.

42. Use according to claim 40, wherein said compound is a drug for the treatment of cancer.

43. Use according to claim 40, wherein said compound is one of the following: Cytochalasin D, Phalloidin, Jasplakinolide, Chondramides, Dolostatin 11, Latrunculin A, Swinholide A, Misakinolide A, Mycalolide B, Shinxolide C, Scytophycins, Goniodomin A, Pseudopterolide, Lysophosphatidylcholine, Lysophosphatidyl acid.

44. A non-human animal wherein at least one of the genes listed in table A, table B, or table 4 is knocked-out, over-expressed or under-expressed.

45. A non-human animal wherein at least one of the genes listed in table A, or table 4 is knocked-out, over-expressed or under-expressed.

46. A non-human animal wherein at least one of the genes listed in table A, table B, or table 4 is knocked out, over-expressed or under-expressed and wherein at least one of the genes listed in table C or table 6 is knocked-out, over-expressed or under-expressed.

47. A non-human animal wherein at least one of the genes listed in table A, or table 4 is knocked out, over-expressed or under-expressed and wherein at least one of the genes listed in table B, in table C or table 6 is knocked-out, over-expressed or under-expressed.

48. A non-human animal wherein at least one gene known to be involved in cytoskeleton rearrangement is knocked-out, over-expressed or under-expressed.

49. A non-human animal according to claim 48, wherein said cytoskeleton arrangement is in a synovial fibroblast.

50. A non-human animal according to claim 48, wherein said gene is involved in extracellular or intracellular signaling to the cytoskeleton.

51. A non-human animal according to claim 48, wherein said gene is involved in binding and/or rearrangement of the actin cytoskeleton.

52. A non-human animal according to claim 48, wherein said gene is any one of the following: tubulin alpha 1, RAB14, Gelsolin, phosphatidylinositol membrane-associated, matrix metalloproteinase 3, matrix metalloproteinase 13, tissue inhibitor of matrix metalloproteinase 13, cathepsin S, matrix gamma-carboxylglutamate, integrin alpha X, myosin light chain, troponin T I skeletal slow, troponin I skeletal fast 2, VASP, ADF/Cofilin and/or is a gene of table 4.

53. A non-human animal according to claim 44, wherein said animal is transgenic for said gene or homologue thereof that is over-expressed or under-expressed.

54. A non-human animal according to claim 44, wherein said animal is a mouse or a rat.

55. A non-human animal according to claim 44, wherein said at least one of the genes listed in table A, table B, table 3, table 4 or table 6 is knocked-out, over-expressed or under-expressed, in a genetic model for arthritis.

56. A non-human animal according to claim 55, wherein said genetic model 10 comprises the specific genetic modification of a Tg197 mouse or a of genetic model as depicted in table 5.

57. Use of a non-human animal according to claim 44, as a disease model.

58. Use according to claim 57, wherein said disease is inflammatory arthritis, and preferably rheumatoid arthritis.

59. Use according to claim 58, wherein said inflammatory and/or rheumatoid arthritis is induced.

60. Use according to claim 59, wherein said induction is achieved using an inducer as depicted in table 5.

61. A method for determining whether a compound is effective in alleviating a symptom of inflammatory arthritis, comprising testing said compound in a model system for said symptom, said method characterized in that said compound is a compound for altering the cytoskeleton or its organization in a cell.

62. A method according to claim 61, wherein said compound is effective in cancer therapy.

63. A method according to claim 61, wherein said compound is one of the following: Cytochalasin D, Phalloidin, Jasplakinolide, Chondramides, Dolostatin 11, Latrunculin A, Swinholide A, Misakinolide A, Mycalolide B, Shinxolide C, Scytophycins, Goniodomin A, Peeudopterolide, Lysophosphatidylcholine, Lysophosphatidyl acid.

64. A method for determining whether a compound has an effect on an expression of one or more of the genes listed in table A, table B, or table 4, comprising testing said compound in an environment wherein said gene is expressed and comparing a resulting expression level with a reference value.

65. A method for determining whether a compound has an effect on an expression of one or more of the genes listed in table A, or table 4, comprising testing said compound in an environment wherein said gene is expressed and comparing a resulting expression level with a reference value.

66. A method for determining whether a compound has an effect on an expression of one or more of the genes listed in table A, table B, or table 4 and on one or more of the genes listed in table C, comprising testing said compound in an environment wherein said genes are expressed and comparing a resulting expression level with a reference value.

67. A method for determining whether a compound has an effect on an expression of one or more of the genes listed in table A, or table 4 and on one or more of the genes listed in table B or in table C, comprising testing said compound in an environment wherein said genes are expressed and comparing a resulting expression level with a reference value.

68. A method for alleviating at least one symptom of inflammatory and/or rheumatoid arthritis in an individual, comprising modifying the level of expression of at least one gene product of a gene of table A, table B, or table 4 in cells of said individual, wherein said gene product comprises protein.

69. Use of a compound for the manufacture of a medicament for alleviating at least one symptom of inflammatory and/or rheumatoid arthritis in an individual, said compound comprising an antisense-sequence of one of the genes listed in table A, table B, or table 4.

70. Use according to claim 69, wherein said gene is identified in either of said tables by expression SF Up or WJ Up or said gene is VASP.

71. Use according to claim 69, wherein said compound comprises an antisense-sequence of one of the genes listed in table A, or table 4.

72. Use according to claim 69, wherein said compound comprises an antisense-sequence of one of the genes listed in table A or table B and an antisense-sequence of one of the genes listed in table C.

73. Use according to claim 69, wherein said compound comprises an antisense-sequence of one of the genes listed in table A and an antisense-sequence of one of the genes listed in table B or in table C.

74. Use of a protein encoded by a gene of table A, table B, or table 4 as a drug target for selecting candidate drugs for a treatment of inflammatory and/or rheumatoid arthritis.

75. Use according to claim 74, wherein said protein is encoded by a gene of table A.

76. Use according to claim 74, wherein a first protein encoded by a gene of table A or table B is used as a first drug target and wherein a second protein encoded by a gene of table C is used as a second drug target.

77. Use according to claim 74, wherein a first protein encoded by a gene of table A is used as a first drug target and wherein a second protein encoded by a gene of table B or table C is used as a second drug target.

78. Use according to claim 74, wherein said gene is identified in either of said tables by expression SF Up or WJ Up or said gene is VASP.


SEQUENCE LISTING (66 sequences)

SEQ ID NO: 1 - DNA, 20 bp, Artificial Sequence
  Synthetic Primer sequence Aqp1 (sense)
tcacccgcaacttctcaaac

SEQ ID NO: 2 - DNA, 20 bp, Artificial Sequence
  Synthetic Primer Aqp1 (antisense)
agctctgagaccaggaaaca

SEQ ID NO: 3 - DNA, 20 bp, Artificial Sequence
  Synthetic Primer Bsg (sense)
atgagaagaggcggaagcca

SEQ ID NO: 4 - DNA, 20 bp, Artificial Sequence
  Synthetic Primer - Bsg (antisense)
ccactccacagggctgtagt

SEQ ID NO: 5 - DNA, 20 bp, Artificial Sequence
  Synthetic Primer CD14 (sense)
caatcctgaattgggcgaga

SEQ ID NO: 6 - DNA, 20 bp, Artificial Sequence
  Synthetic Primer CD14 (antisense)
cgagtgggattcagagtcca

SEQ ID NO: 7 - DNA, 20 bp, Artificial Sequence
  Synthetic Primer CDC42 (sense)
aagtggcccagatcctggaa

SEQ ID NO: 8 - DNA, 20 bp, Artificial Sequence
  Synthetic Primer CDC42 (antisense)
agcactgcacttttggggtt

SEQ ID NO: 9 - DNA, 20 bp, Artificial Sequence
  Synthetic Primer Gsn (sense)
tgcaggaagacctggctact

SEQ ID NO: 10 - DNA, 20 bp, Artificial Sequence
  Synthetic Primer Gsn (antisense)
atggcttggtccttactcag

SEQ ID NO: 11 - DNA, 20 bp, Artificial Sequence
  Synthetic Primer Hp (sense)
gaagcaatgggtgaacacag

SEQ ID NO: 12 - DNA, 20 bp, Artificial Sequence
  Synthetic Primer Hp (antisense)
ggggtggagaacgaccttct

SEQ ID NO: 13 - DNA, 20 bp, Artificial Sequence
  Synthetic Primer Marco (sense)
cacaggaattcaaggacaga

SEQ ID NO: 14 - DNA, 20 bp, Artificial Sequence
  Synthetic Primer Marco (antisense)
attgtccagccagatgttcc

SEQ ID NO: 15 - DNA, 20 bp, Artificial Sequence
  Synthetic Primer Mglap (sense)
cagtcccttcatcaacagga

SEQ ID NO: 16 - DNA, 20 bp, Artificial Sequence
  Synthetic Primer Mglap (antisense)
ctgcaggagatataaaacga

SEQ ID NO: 17 - DNA, 20 bp, Artificial Sequence
  Synthetic Primer Mb (sense)
tcacacgccaccaagcacaa

SEQ ID NO: 18 - DNA, 20 bp, Artificial Sequence
  Synthetic Primer Mb (antisense)
tgggctccagggtaacactg

SEQ ID NO: 19 - DNA, 20 bp, Artificial Sequence
  Synthetic Primer Ptgis (sense)
tcacagatgaccacactccc

SEQ ID NO: 20 - DNA, 20 bp, Artificial Sequence
  Synthetic Primer Ptgis (antisense)
gcagtaggacgacaaattgt

SEQ ID NO: 21 - DNA, 21 bp, Artificial Sequence
  Synthetic Primer B2M (sense)
ttctggtgcttgtctcactga

SEQ ID NO: 22 - DNA, 22 bp, Artificial Sequence
  Synthetic Primer B2M (antisense)
cagtatgttcggcttcccattc

SEQ ID NO: 23 - DNA, 21 bp, Artificial Sequence
  Synthetic Primer L32 (sense)
ttaagcgaaactggcggaaac

SEQ ID NO: 24 - DNA, 21 bp, Artificial Sequence
  Synthetic Primer L32 (antisense)
ttgttgctcccataaccgatg

SEQ ID NO: 25 - DNA, 20 bp, Artificial Sequence
  Synthetic Primer mouse Aqp1
tcacccgcaacttctcaaac

SEQ ID NO: 26 - DNA, 20 bp, Artificial Sequence
  Synthetic Primer mouse Aqp1 (antisense)
tcatgcggtctgtgaagtcg

SEQ ID NO: 27 - DNA, 20 bp, Artificial sequence
  Synthetic Primer Mouse Gsn (sense)
tgcaggaagacctggctact

SEQ ID NO: 28 - DNA, 20 bp, Artificial Sequence
  Synthetic Primer mouse Gsn (antisense)
tcgatgtaccgcttagcaga

SEQ ID NO: 29 - DNA, 19 bp, Artificial Sequence
  Synthetic Primer human Aqp1(sense)
ctccctgactgggaactcg

SEQ ID NO: 30 - DNA, 19 bp, Artificial sequence
  Synthetic Primer human Aqg1
gggctacagagaggccgat

SEQ ID NO: 31 - DNA, 20 bp, Artificial Sequence
  Synthetic Primer human Bsg
ttcctgggcatcgtggctga

SEQ ID NO: 32 - DNA, 20 bp, Artificial Sequence
  Synthetic Primer human Bsg (antisense)
gcggacgttcttgcctttgt

SEQ ID NO: 33 - DNA, 19 bp, Artificial Sequence
  Synthetic Primer human Cd14 (sense)
cggcggtctcaacctagag

SEQ ID NO: 34 - DNA, 20 bp, Artificial Sequence
  Synthetic Primer human Cd14 (antisense)
gcctaccagtagctgagcag

SEQ ID NO: 35 - DNA, 20 bp, Artificial Sequence
  Synthetic Primer human cdc42 (sense)
aattgatctcagagatgacc

SEQ ID NO: 36 - DNA, 20 bp, Artificial Sequence
  Synthetic Primer human cdc42 (antisense)
tttaggcctttctgtgtaag

SEQ ID NO: 37 - DNA, 21 bp, Artificial Sequence
  Synthetic Primer human Gsn (sense)
ggtgtggcatcaggattcaag

SEQ ID NO: 38 - DNA, 22 bp, Artificial Sequence
  Synthetic Primer human Gsn (antisense)
tttcataccgattgctgttgga

SEQ ID NO: 39 - DNA, 19 bp, Artificial Sequence
  Synthetic Primer human Marco (sense)
tgggacgagatggagcaac

SEQ ID NO: 40 - DNA, 22 bp, Artificial Sequence
  Synthetic Primer human Marco (antisense)
cccttagttccagtttcccctt

SEQ ID NO: 41 - DNA, 19 bp, Artificial Sequence
  Synthetic Primer human Mb (sense)
ttggtgctgaacgtctggg

SEQ ID NO: 42 - DNA, 21 bp, Artificial Sequence
  Synthetic Primer human Mb (antisense)
ctgtgccaggggcttaatctc

SEQ ID NO: 43 - DNA, 20 bp, Artificial Sequence
  Synthetic Primer human B2M (sense)
ctgaaaaagatgagtatgcc

SEQ ID NO: 44 - DNA, 20 bp, Artificial Sequence
  Synthetic Primer human B2M (antisense)
accctacattttgtgcataa

SEQ ID NO: 45 - DNA, 21 bp, Artificial Sequence
  Synthetic Primer human L32 (sense)
ttaagcgtaactggcggaaac

SEQ ID NO: 46 - DNA, 20 bp, Artificial Sequence
  Synthetic Primer human L32
gagcgatctcggcacagtaa

SEQ ID NO: 47 - DNA, 20 bp, Artificial Sequence
  Synthetic Primer mouse Cofilin 1 (sense)
tgggtgaaactctgggagat

SEQ ID NO: 48 - DNA, 20 bp, Artificial Sequence
  Synthetic Primer mouse Cofilin 1 (antisense)
ccatagtggaagcctgatgc

SEQ ID NO: 49 - DNA, 19 bp, Artificial Sequence
  Synthetic Primer mouse VASP (sense)
cttgccaaggatgaagtcg

SEQ ID NO: 50 - DNA, 18 bp, Artificial Sequence
  Synthetic Primer mouse VASP
gcttcaccctctccaagt

SEQ ID NO: 51 - DNA, 21 bp, Artificial Sequence
  Synthetic Primer human C3 (sense 5′-3′)
ggcctttgttctcatctcgct

SEQ ID NO: 52 - DNA, 21 bp, Artificial Sequence
  Synthetic Primer human C3 (antisense 5′-3′)
ataggatgtggcctccacgtt

SEQ ID NO: 53 - DNA, 22 bp, Artificial Sequence
  Synthetic primer human CSTB (sense 5′-3′)
cctgtgtttaaggccgtgtcat

SEQ ID NO: 54 - DNA, 22 bp, Artificial Sequence
  Synthetic Primer human CSTB (antisense 5′-3′)
ggagagattggaacactcgcag

SEQ ID NO: 55 - DNA, 22 bp, Artificial Sequence
  Synthetic Primer human DUSP1 (sense 5′-3′)
acaaggcagacatcagctcctg

SEQ ID NO: 56 - DNA, 21 bp, Artificial Sequenc
  Synthetic Primer human DUSP1 (antisense 5′-3′)
taagcaaggcagatggtggct

SEQ ID NO: 57 - DNA, 22 bp, Artificial Sequence
  Synthetic Primer human LSP1 (sense 5′-3′)
caacaattaagagcaccccatc

SEQ ID NO: 58 - DNA, 21 bp, Artificial Sequence
  Synthetic Primer human LSP1 (antisense 5′-3′)
tggcaacaggaagcaacttct

SEQ ID NO: 59 - DNA, 22 bp, Artificial Sequence
  Synthetic Primer human RAB14
gcttattgttcctcgaagcgag

SEQ ID NO: 60 - DNA, 22 bp, Artificial Sequence
  Synthetic Primer human RAB14 (antisense 5′-3′)
atccaagcttccatcctgaatg

SEQ ID NO: 61 - DNA, 22 bp, Artificial Sequence
  Synthetic Primer human SHIP2
ggctcggaggaaccaaaactac

SEQ ID NO: 62 - DNA, 21 bp, Artificial Sequence
  Synthetic Primer human SHIP2 (sense 5′-3′)
tgaggtccccaaaccagaaga

SEQ ID NO: 63 - DNA, 22 bp, Artificial Sequence
  Synthetic Primer human TGFB1I4 (sense 5′-3′)
tagctcctctggtgcaagtgtg

SEQ ID NO: 64 - DNA, 21 bp, Artificial Sequence
  Synthetic Primer human TGFB1I4 (antisense 5′-3′)
tccacttcttctctgaccgca

SEQ ID NO: 65 - DNA, 19 bp, Artificial Sequence
  Synthetic Primer human LST1 (sense 5′-3′)
cgtgatgaggaacttgagg

SEQ ID NO: 66 - DNA, 20 bp, Artificial Sequence
  Synthetic Primer human LST1 (antisense 5′-3′)
gcgataacattaggtcaggg